(19) United States
(12) Patent Application Publication
(10) Pub. No.: US 2016/0204346 A1
(43) Pub. Date: Jul. 14, 2016

(54) COMPOUND AND ORGANIC LIGHT-EMITTING DEVICE INCLUDING THE SAME

(71) Applicant: SAMSUNG DISPLAY CO., LTD., Yongin-City (KR)

(72) Inventors: Sanghyun Han, Yongin-City (KR); Youngkook Kim, Yongin-City (KR); Hyejin Jung, Yongin-City (KR); Seokhwan Hwang, Yongin-City (KR); Hyoungkun Kim, Yongin-City (KR)

(21) Appl. No.: 14/788,059

(22) Filed: Jun. 30, 2015

(30) Foreign Application Priority Data
Jan. 9, 2015 (KR) 10-2015-0003467

(51) Int. Cl. H01L 51/00; H01L 27/32; C09K 11/06; C07C 211/61; C07D 307/91

(52) U.S. Cl. CPC H01L 51/006; C07C 211/61; C07D 307/91; C09K 11/06; H01L 51/0061; H01L 51/0077; H01L 27/3248; C09K 2211/1014; C09K 2211/1011; C09K 2211/1007; C09K 2211/1088; C09K 2211/1022; H01L 51/0058; H01L 51/0073; H01L 51/5012

(57) ABSTRACT

A compound represented by Formula 1.

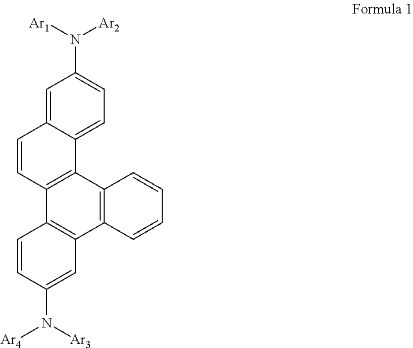

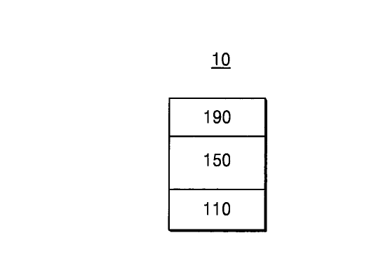

An organic light-emitting device includes a first electrode; a second electrode facing the first electrode; and an organic layer between the first electrode and the second electrode, the organic layer including an emission layer, wherein the emission layer includes the compound of Formula 1 as a dopant. The organic light-emitting device including the compound of Formula 1 may have high efficiency, low voltage, high brightness, and/or long lifespan.

### CROSS-REFERENCE TO RELATED APPLICATION

This application claims priority to and the benefit of Korean Patent Application No. 10-2015-0003467, filed on Jan. 9, 2015, in the Korean Intellectual Property Office, the entire content of which is incorporated herein by reference.

### BACKGROUND

1. Field

One or more aspects of embodiments of the present invention are directed to a compound and an organic light-emitting device including the compound.

2. Description of the Related Art

Organic light-emitting devices are self-emission devices that have wide viewing angles, high contrast ratios, short response time, and excellent brightness, driving voltage, and response speed characteristics, and can produce multi-colored images.

The organic light-emitting device may have a structure including a first electrode positioned on a substrate, and a hole transport region, an emission layer, an electron transport region, and a second electrode sequentially stacked on the first electrode. Holes provided from the first electrode may move toward the emission layer through the hole transport region, and electrons provided from the second electrode may move toward the emission layer through the electron transport region. Carriers (for example, holes and electrons), may be recombined in the emission layer to produce excitons. These excitons may change from an excited state to a ground state, thereby generating light.

### SUMMARY

One or more aspects of embodiments of the present invention are directed to a blue fluorescent dopant compound having high efficiency and/or improved long lifespan characteristics and an organic light-emitting device including the blue fluorescent dopant compound.

Additional aspects will be set forth in part in the description which follows and, in part, will be apparent from the description, or may be learned by practice of the presented embodiments.

According to one or more embodiments of the present invention, there is provided a compound represented by Formula 1 below:

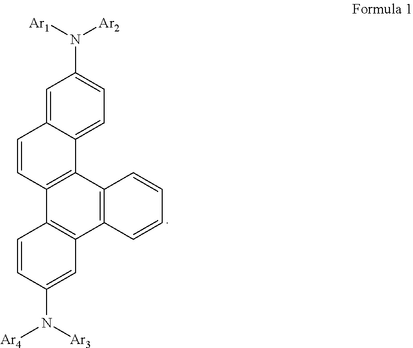

In Formula 1,

Ar₁ to Ar₄ may each independently be selected from a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, a hydroxyl group, a cyano group, a nitro group, an amino group, an amidino group, a hydrazine group, a hydrazone group, a carboxylic acid (herein, also referring to a carboxylic acid group) or a salt thereof, a sulfonic acid (herein, also referring to a sulfonic acid group) or a salt thereof, a phosphoric acid (herein, also referring to a phosphoric acid group) or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group (aryloxy), C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), and —B(Q₁₆)(Q₁₇);

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅), and —B(Q₃₆)(Q₃₇),

where Q₁₁ to Q₁₇, Q₂₁ to Q₂₇, and Q₃₁ to Q₃₇ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

According to one or more embodiments of the present invention, an organic light-emitting device includes a first electrode; a second electrode facing the first electrode; an organic layer between the first electrode and the second electrode and including an emission layer, wherein the organic layer includes the compound of Formula 1 above.

According to one or more embodiments of the present invention, a flat panel display apparatus includes the organic light-emitting device, wherein the first electrode of the organic light-emitting device is electrically connected to a source electrode or a drain electrode of a thin film transistor.

### DETAILED DESCRIPTION OF THE DRAWINGS

These and/or other aspects will become apparent and more readily appreciated from the following description of embodiments of the present invention, taken in conjunction with the drawing, which is a schematic view of an organic light-emitting device according to one or more embodiments.

### DETAILED DESCRIPTION

Reference will now be made in more detail to embodiments, examples of which are illustrated in the accompanying drawing, wherein like reference numerals refer to like elements throughout. In this regard, the present embodiments may have different forms and should not be construed as being limited to the descriptions set forth herein. Accordingly, the present embodiments are merely described below, by referring to the drawing, to explain aspects of the present description. As used herein, the term “and/or” includes any and all combinations of one or more of the associated listed items. Expressions such as “at least one of”, when preceding a list of elements, modify the entire list of elements and do not modify the individual elements of the list. Further, the use of “may” when describing embodiments of the present invention refers to “one or more embodiments of the present invention.”

In addition, as used herein, the terms “use,” “using,” and “used” may be considered synonymous with the terms “utilize,” “utilizing,” and “utilized,” respectively. Also, the term “exemplary” is intended to refer to an example or illustration.

As used herein, the term “substantially,” “about,” and similar terms are used as terms of approximation and not as terms of degree, and are intended to account for the inherent deviations in measured or calculated values that would be recognized by those of ordinary skill in the art.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. §112, first paragraph, and 35 U.S.C. §132(a).

According to one or more aspects of embodiments of the present invention, there is provided a compound represented by Formula 1:

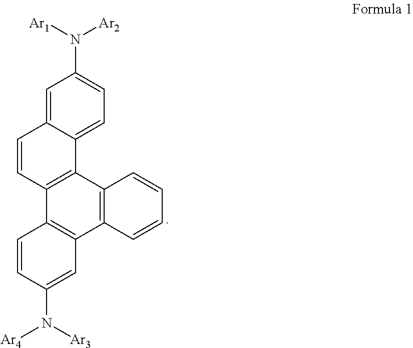

In Formula 1,

Ar₁ to Ar₄ may each independently be selected from a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group (aryloxy), C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), and —B(Q₁₆)(Q₁₇);

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅), and —B(Q₃₆)(Q₃₇),

where Q₁₁ to Q₁₇, Q₂₁ to Q₂₇, and Q₃₁ to Q₃₇ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

In related art, a blue light-emitting compound in which a diphenylanthracene structure is included in the core and an aryl group is substituted at the end of the compound is a known blue light-emission material, and an organic light-emitting device including the blue light-emitting compound is also known in related art. However, such blue light-emitting compound and the organic light-emitting device including the blue light-emitting compound may have insufficient light-emitting efficiency and brightness.

Similarly, an organic light-emitting device including a substituted pyrene-based compound is also known in the related art. However, such organic light-emitting device may have difficulty in implementing deep blue light due to low blue color purity, which in turn leads to difficult implementation of a full, natural color display.

According to one or more embodiments of the present invention, there is provided a novel compound and an organic light-emitting device including the novel compound having significantly improved characteristics as compared with related organic light-emitting devices.

The novel compound of embodiments of the present invention is a material having excellent electric characteristics, high charge-transporting and light-emitting capabilities, and a high glass transition temperature, and capable of preventing or substantially reducing crystallization. Accordingly, the material may be suitable for fluorescent and phosphorescent devices of all colors including but not limited to red, green, blue, and white. In addition, the organic light-emitting device including the novel compound may have high efficiency, low voltage, high brightness, and long lifespan.

According to one or more embodiments of the present invention, Ar₁ to Ar₄ in Formula 1 may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

According to some embodiments, the compound of Formula 1 may be represented by Formula 2 below:

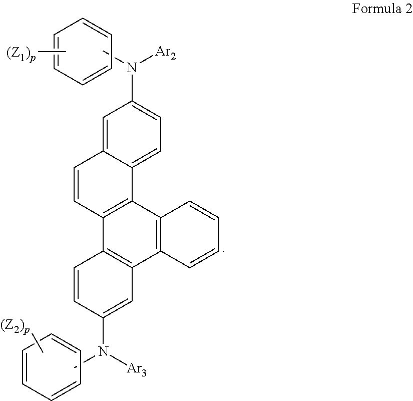

In Formula 2, Z₁ and Z₂ may each independently be selected from hydrogen, deuterium, a halogen atom, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₃)(Q₃₄)(Q₃₅), where Q₃₃ to Q₃₅ are as defined herein;

where a plurality of Z₁ and/or Z₂ exist (where p and/or q is 2 or more), Z₁s and/or Z₂s may be respectively identical to or different from each other;

p and q may each independently be an integer selected from 1 to 5; and

* indicates a binding site.

According to some embodiments, the compound of Formula 1 may be represented by Formula 3 below:

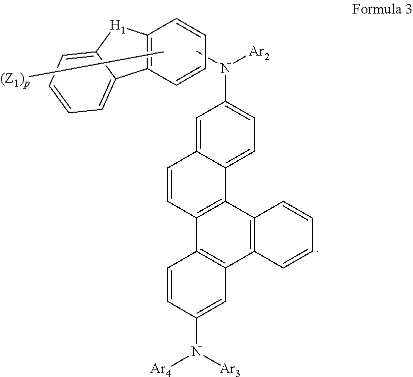

In Formula 3, H₁ may be CR₁R₂, O, or S;

Z₁, R₁, and R₂ may each independently be selected from hydrogen, deuterium, a halogen atom, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₃)(Q₃₄)(Q₃₅), where Q₃₃ to Q₃₅ are as defined herein;

where a plurality of Z₁s exists (where p is 2 or more), Z₁(s) may be identical to or different from each other;

p may be an integer selected from 1 to 7; and

* indicates a binding site.

According to some embodiments, the compound of Formula 1 may be represented by Formula 4 below:

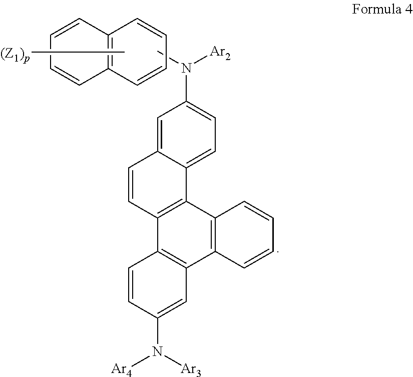

In Formula 4, Z₁ may be selected from hydrogen, deuterium, a halogen atom, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₃)(Q₃₄)(Q₃₅), where Q₃₃ to Q₃₅ are as defined herein;

where a plurality of Z₁s exists (where p is 2 or more), Z₁(s) may be identical to or different from each other;

p may be an integer selected from 1 to 7; and

* indicates a binding site.

According to some embodiments, the compound of Formula 1 may be represented by Formula 5 below:

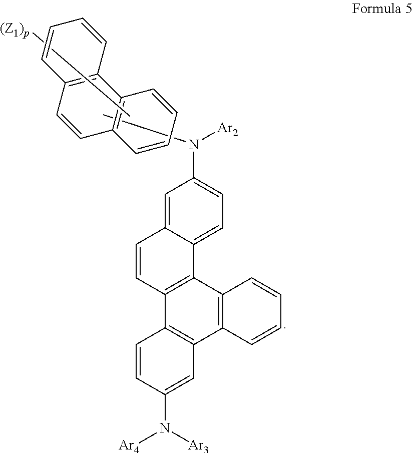

In Formula 5, Z₁ may be selected from hydrogen, deuterium, a halogen atom, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₃)(Q₃₄)(Q₃₅), where Q₃₃ to Q₃₅ are as defined herein;

where a plurality of Z₁s exists (where p is 2 or more), Z₁(s) may be identical to or different form each other;

p may be an integer selected from 1 to 9; and

* indicates a binding site.

According to some embodiments, Ar₁ to Ar₄ may each independently be selected from hydrogen, deuterium, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group; and

a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group, each substituted with at least one selected from deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, —Si(Q₃₃)(Q₃₄)(Q₃₅), C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group and a dibenzocarbazolyl group, a thiadiazolyl group, an imidazopyridinyl group, and an imidazopyrimidinyl group.

According to some embodiments, Ar₁ to Ar₄ may each independently be selected from hydrogen, deuterium, and a compound represented by any one of Formulae 2a to 2e below:

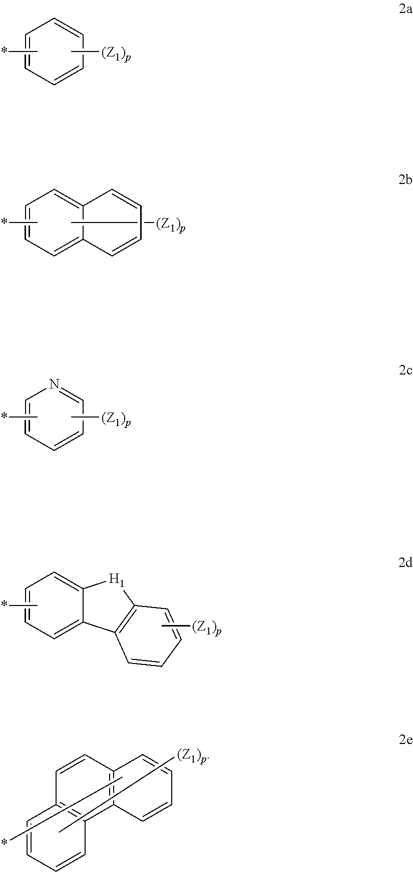

In Formulae 2a to 2e, H₁ may be CR₁R₂, O, or S;

Z₁, R₁, and R₂ may each independently be selected from hydrogen, deuterium, a halogen atom, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₃)(Q₃₄)(Q₃₅), where Q₃₃ to Q₃₅ are as defined herein;

where a plurality of Z₁s exists (where p is 2 or more), Z₁(s) may be identical to or different from each other;

p may be an integer selected from 1 to 9; and

* indicates a binding site.

According to some embodiments, the compound of Formula 1 may be selected from Compounds 1-112 below:

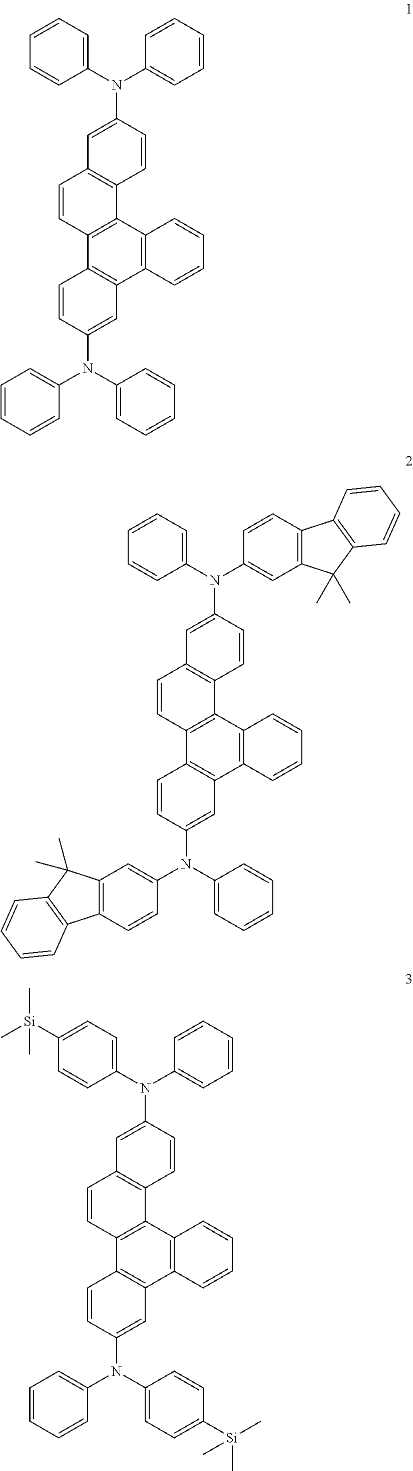

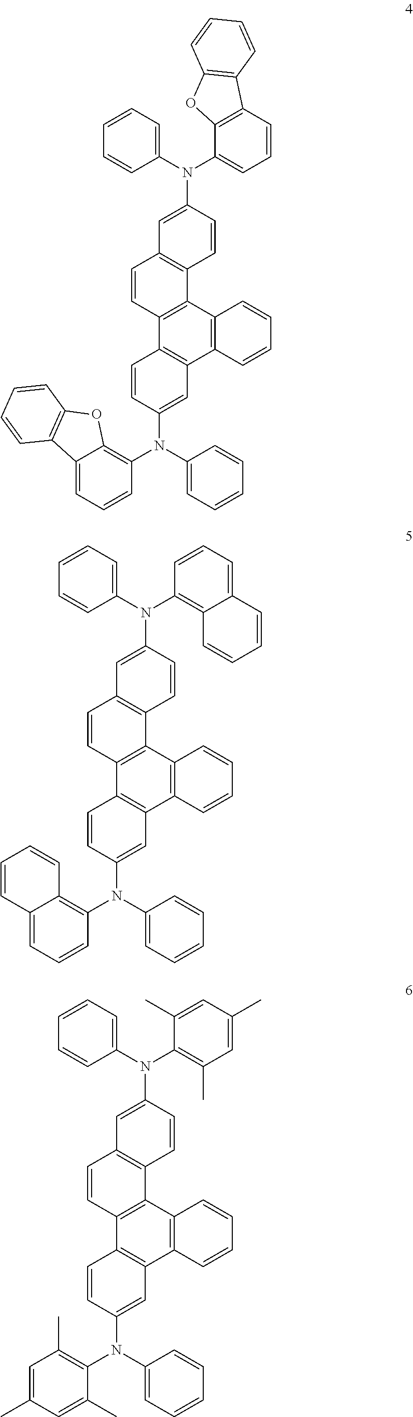

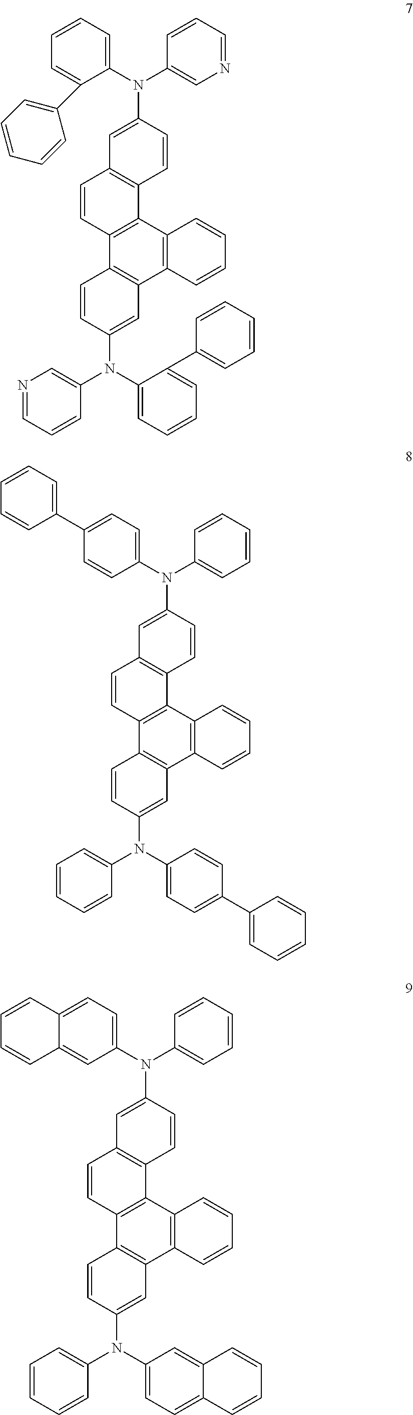

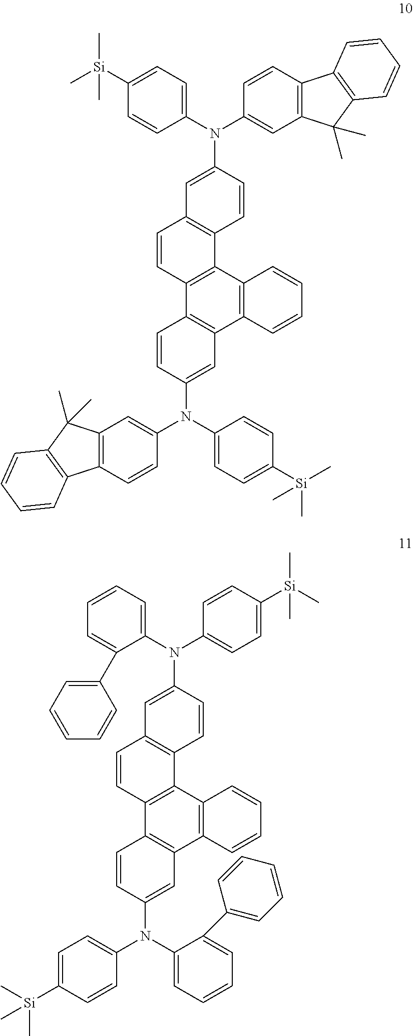

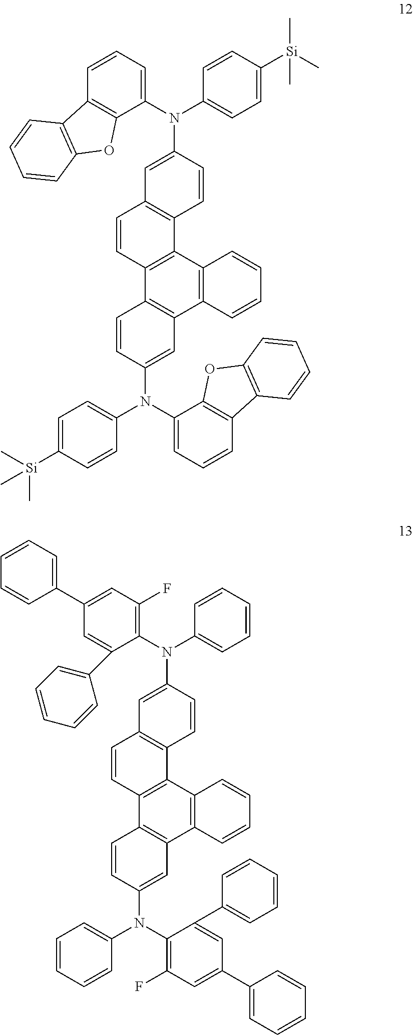

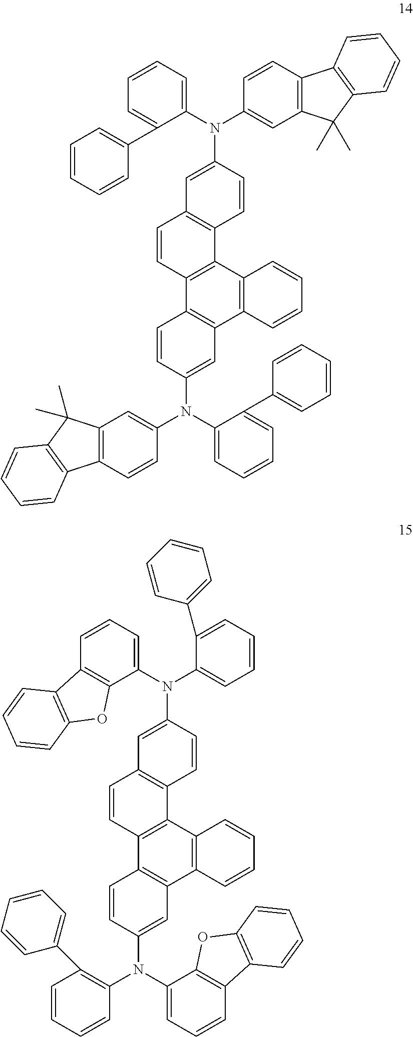 [SMILES: CC1(C)c2ccccc2-c2ccc(N(c3ccc4c(ccc5c6ccc(N(c7ccc8c(c7)C(C)(C)c7ccccc7-8)c7ccccc7-c7ccccc7)cc6c6ccccc6c45)c3)c3ccccc3-c3ccccc3)cc21 ; c1ccc(-c2ccccc2N(c2ccc3c(ccc4c5ccc(N(c6ccccc6-c6ccccc6)c6cccc7c6oc6ccccc67)cc5c5ccccc5c34)c2)c2cccc3c2oc2ccccc23)cc1]

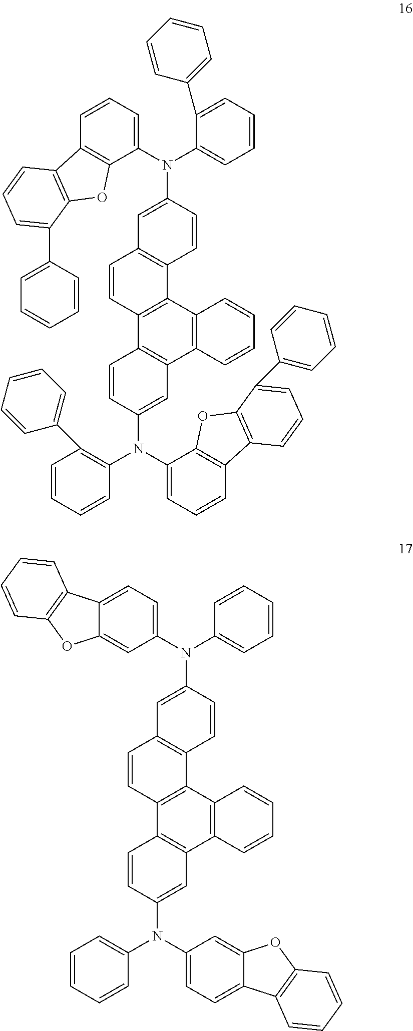 [SMILES: c1ccc(-c2ccccc2N(c2ccc3c(ccc4c5ccc(N(c6ccccc6-c6ccccc6)c6cccc7c6oc6c(-c8ccccc8)cccc67)cc5c5ccccc5c34)c2)c2cccc3c2oc2c(-c4ccccc4)cccc23)cc1 ; c1ccc(N(c2ccc3c(ccc4c5ccc(N(c6ccccc6)c6ccc7c(c6)oc6ccccc67)cc5c5ccccc5c34)c2)c2ccc3c(c2)oc2ccccc23)cc1]

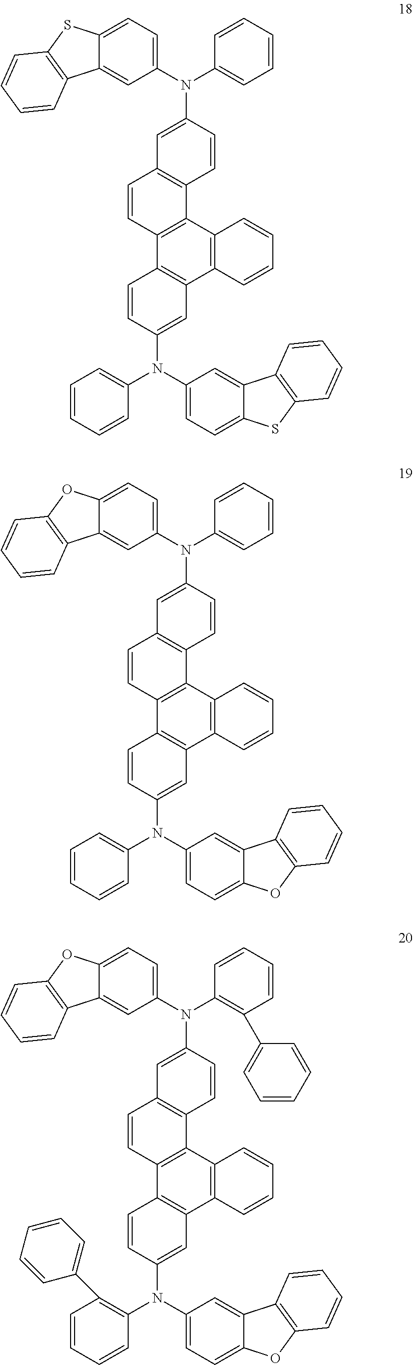 [SMILES: c1ccc(N(c2ccc3c(ccc4c5ccc(N(c6ccccc6)c6ccc7sc8ccccc8c7c6)cc5c5ccccc5c34)c2)c2ccc3sc4ccccc4c3c2)cc1 ; c1ccc(N(c2ccc3c(ccc4c5ccc(N(c6ccccc6)c6ccc7oc8ccccc8c7c6)cc5c5ccccc5c34)c2)c2ccc3oc4ccccc4c3c2)cc1 ; c1ccc(-c2ccccc2N(c2ccc3c(ccc4c5ccc(N(c6ccc7oc8ccccc8c7c6)c6ccccc6-c6ccccc6)cc5c5ccccc5c34)c2)c2ccc3oc4ccccc4c3c2)cc1]

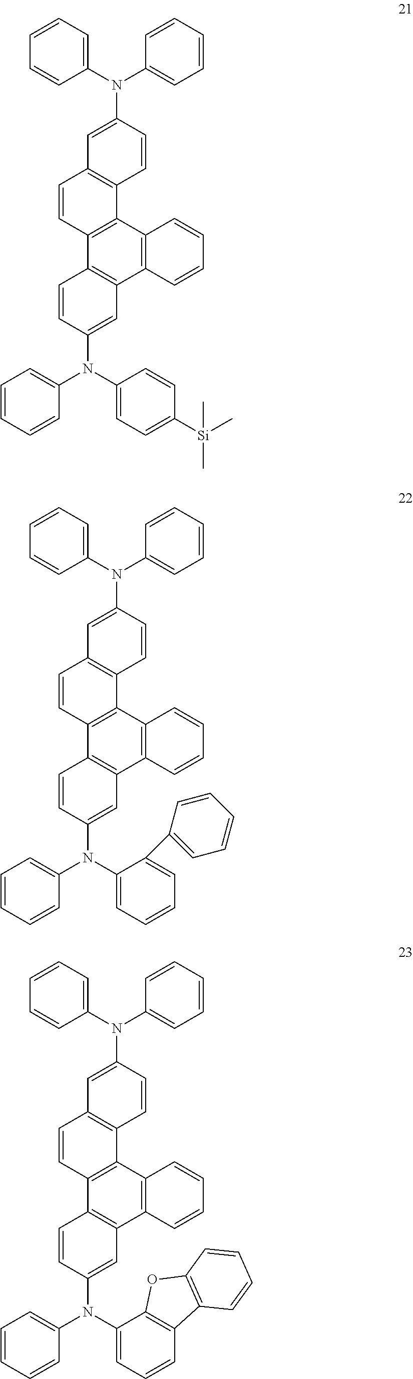 [SMILES: C[Si](C)(C)c1ccc(N(c2ccccc2)c2ccc3c(c2)c2ccccc2c2c4ccc(N(c5ccccc5)c5ccccc5)cc4ccc32)cc1 ; c1ccc(-c2ccccc2N(c2ccccc2)c2ccc3c(c2)c2ccccc2c2c4ccc(N(c5ccccc5)c5ccccc5)cc4ccc32)cc1 ; c1ccc(N(c2ccccc2)c2ccc3c(ccc4c5ccc(N(c6ccccc6)c6cccc7c6oc6ccccc67)cc5c5ccccc5c34)c2)cc1]

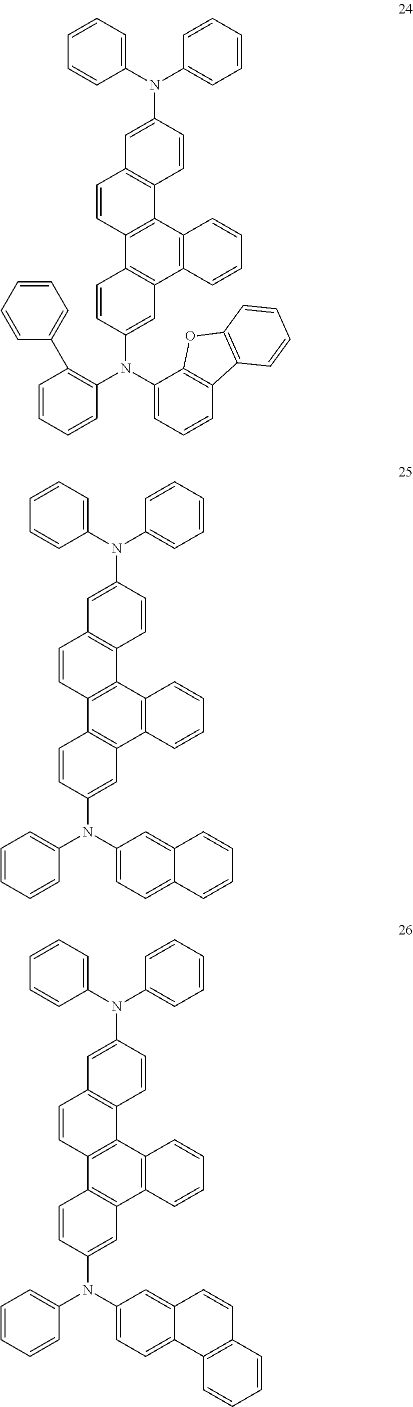 [SMILES: c1ccc(-c2ccccc2N(c2ccc3c(c2)c2ccccc2c2c4ccc(N(c5ccccc5)c5ccccc5)cc4ccc32)c2cccc3c2oc2ccccc23)cc1 ; c1ccc(N(c2ccccc2)c2ccc3c(ccc4c5ccc(N(c6ccccc6)c6ccc7ccccc7c6)cc5c5ccccc5c34)c2)cc1 ; c1ccc(N(c2ccccc2)c2ccc3c(ccc4c5ccc(N(c6ccccc6)c6ccc7c(ccc8ccccc87)c6)cc5c5ccccc5c34)c2)cc1]

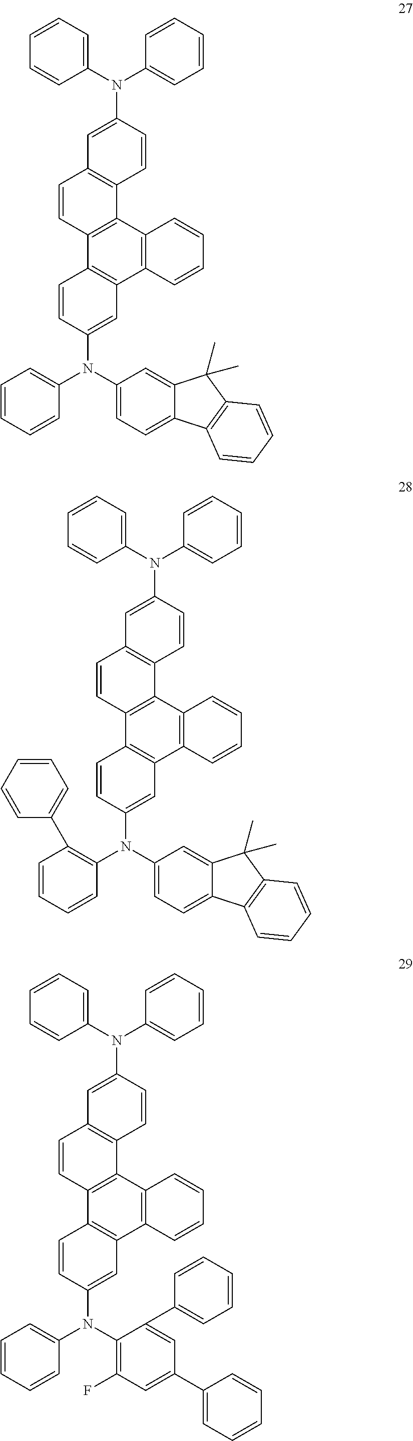

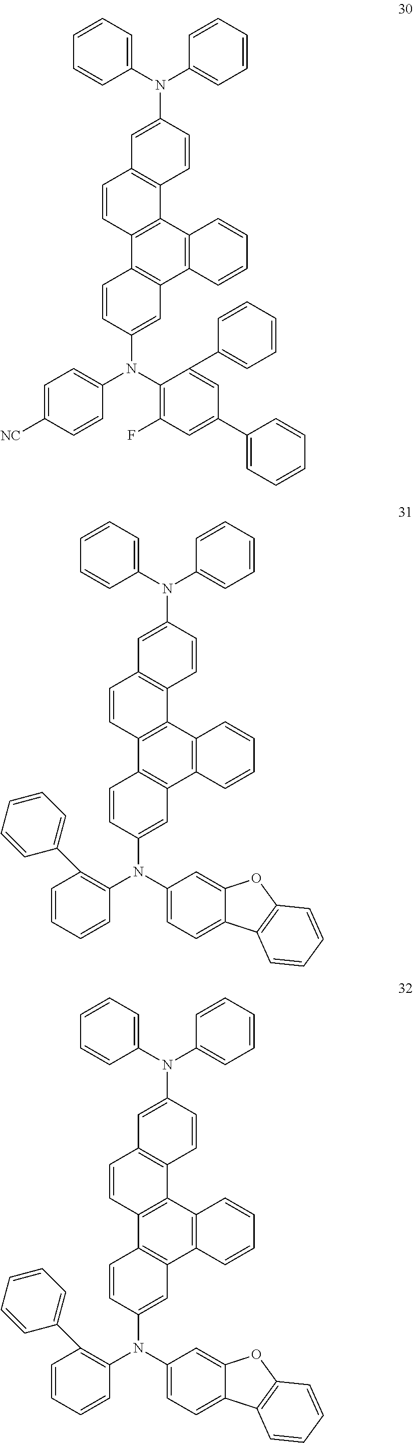 [SMILES: N#Cc1ccc(N(c2ccc3c(c2)c2ccccc2c2c4ccc(N(c5ccccc5)c5ccccc5)cc4ccc32)c2c(F)cc(-c3ccccc3)cc2-c2ccccc2)cc1 ; c1ccc(-c2ccccc2N(c2ccc3c(c2)oc2ccccc23)c2ccc3c(c2)c2ccccc2c2c4ccc(N(c5ccccc5)c5ccccc5)cc4ccc32)cc1 ; c1ccc(-c2ccccc2N(c2ccc3c(c2)oc2ccccc23)c2ccc3c(c2)c2ccccc2c2c4ccc(N(c5ccccc5)c5ccccc5)cc4ccc32)cc1]

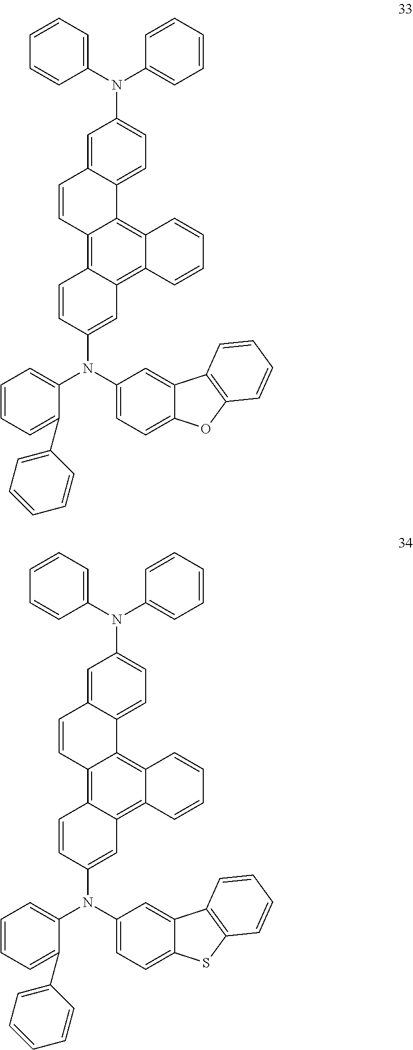

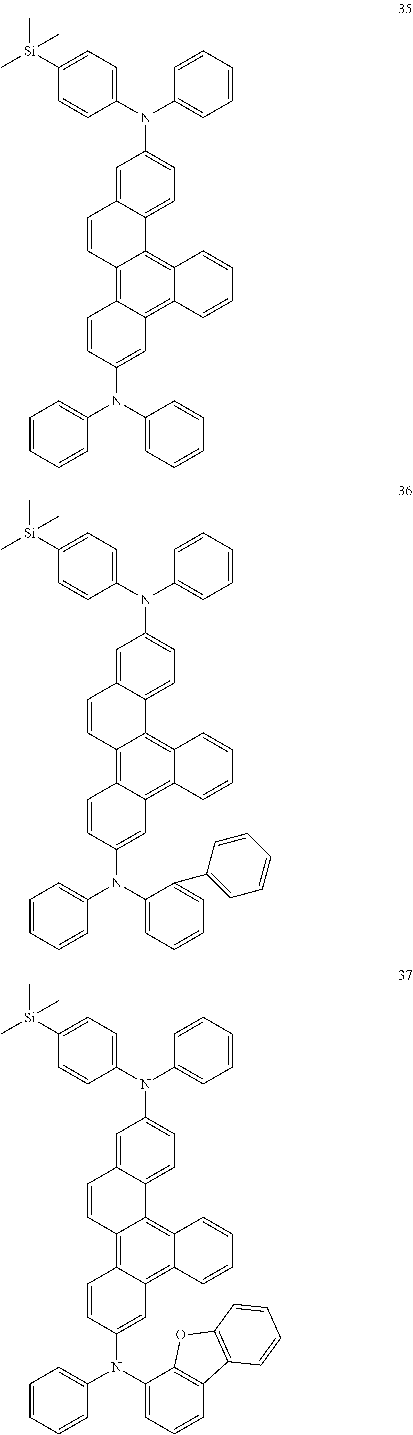

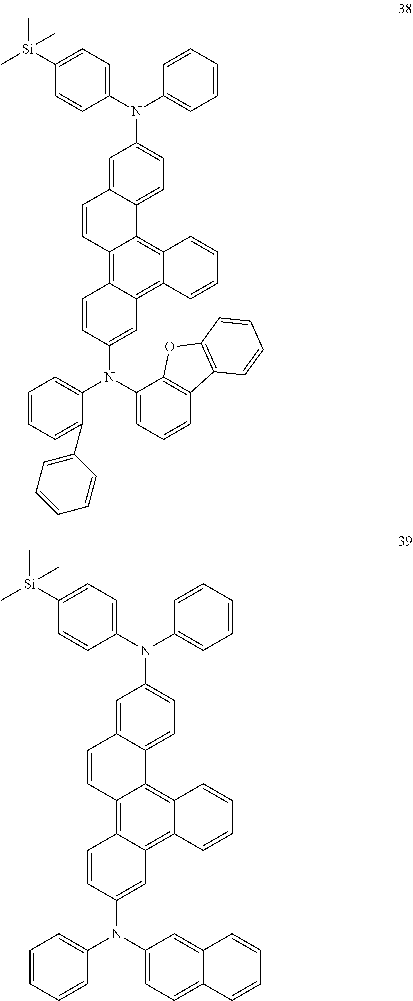 [SMILES: C[Si](C)(C)c1ccc(N(c2ccccc2)c2ccc3c(ccc4c5ccc(N(c6ccccc6-c6ccccc6)c6cccc7c6oc6ccccc67)cc5c5ccccc5c34)c2)cc1 ; C[Si](C)(C)c1ccc(N(c2ccccc2)c2ccc3c(ccc4c5ccc(N(c6ccccc6)c6ccc7ccccc7c6)cc5c5ccccc5c34)c2)cc1]

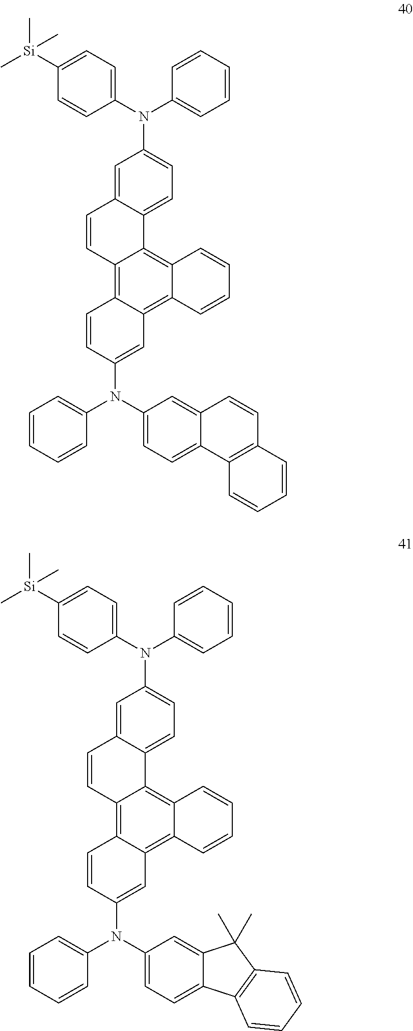 [SMILES: C[Si](C)(C)c1ccc(N(c2ccccc2)c2ccc3c(ccc4c5ccc(N(c6ccccc6)c6ccc7c(ccc8ccccc87)c6)cc5c5ccccc5c34)c2)cc1 ; CC1(C)c2ccccc2-c2ccc(N(c3ccccc3)c3ccc4c(c3)c3ccccc3c3c5ccc(N(c6ccccc6)c6ccc([Si](C)(C)C)cc6)cc5ccc43)cc21]

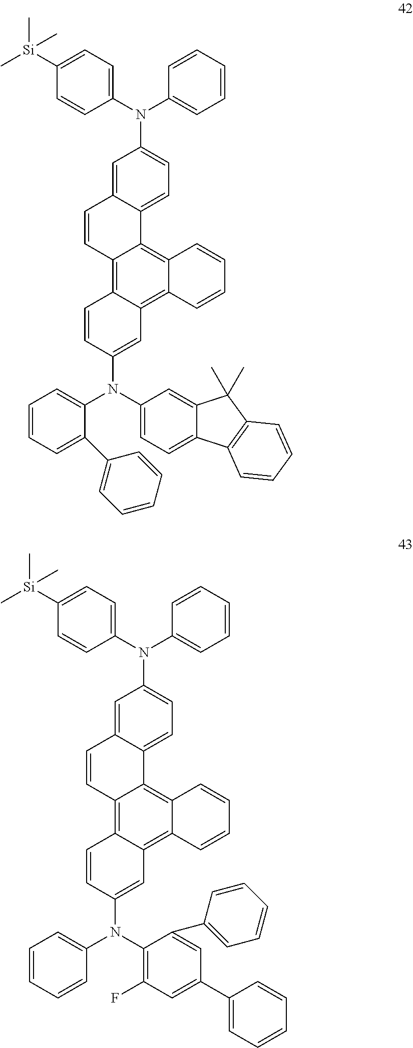 [SMILES: CC1(C)c2ccccc2-c2ccc(N(c3ccc4c(c3)c3ccccc3c3c5ccc(N(c6ccccc6)c6ccc([Si](C)(C)C)cc6)cc5ccc43)c3ccccc3-c3ccccc3)cc21 ; C[Si](C)(C)c1ccc(N(c2ccccc2)c2ccc3c(ccc4c5ccc(N(c6ccccc6)c6c(F)cc(-c7ccccc7)cc6-c6ccccc6)cc5c5ccccc5c34)c2)cc1]

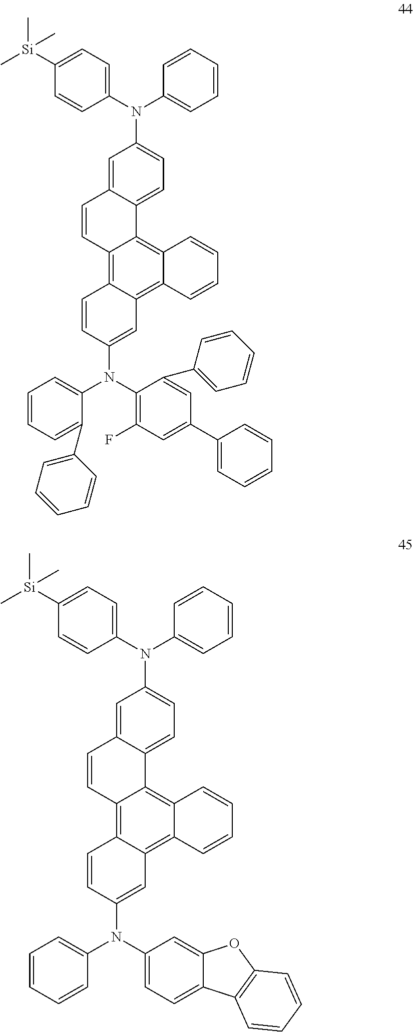 [SMILES: C[Si](C)(C)c1ccc(N(c2ccccc2)c2ccc3c(ccc4c5ccc(N(c6ccccc6-c6ccccc6)c6c(F)cc(-c7ccccc7)cc6-c6ccccc6)cc5c5ccccc5c34)c2)cc1 ; C[Si](C)(C)c1ccc(N(c2ccccc2)c2ccc3c(ccc4c5ccc(N(c6ccccc6)c6ccc7c(c6)oc6ccccc67)cc5c5ccccc5c34)c2)cc1]

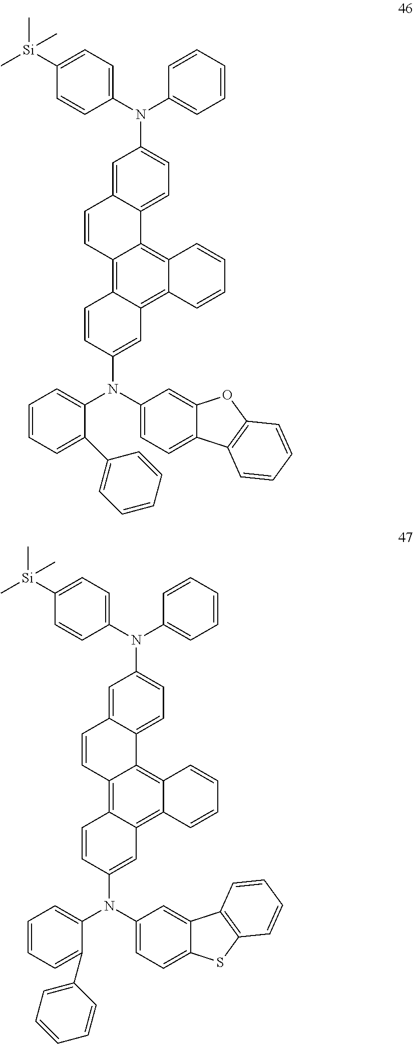 [SMILES: C[Si](C)(C)c1ccc(N(c2ccccc2)c2ccc3c(ccc4c5ccc(N(c6ccc7c(c6)oc6ccccc67)c6ccccc6-c6ccccc6)cc5c5ccccc5c34)c2)cc1 ; C[Si](C)(C)c1ccc(N(c2ccccc2)c2ccc3c(ccc4c5ccc(N(c6ccc7sc8ccccc8c7c6)c6ccccc6-c6ccccc6)cc5c5ccccc5c34)c2)cc1]

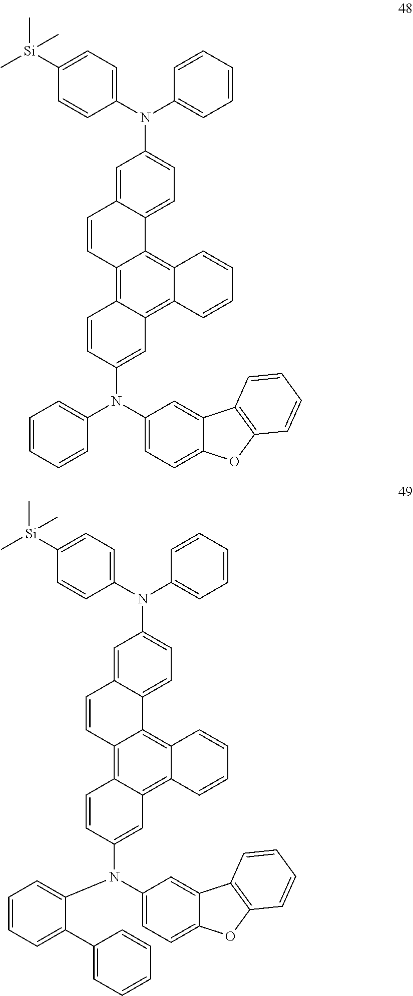

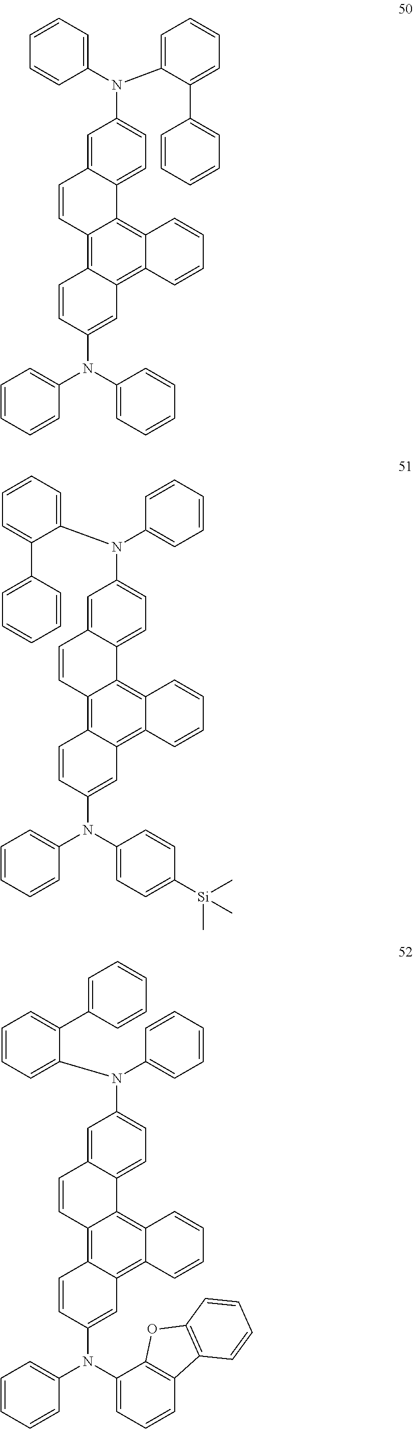

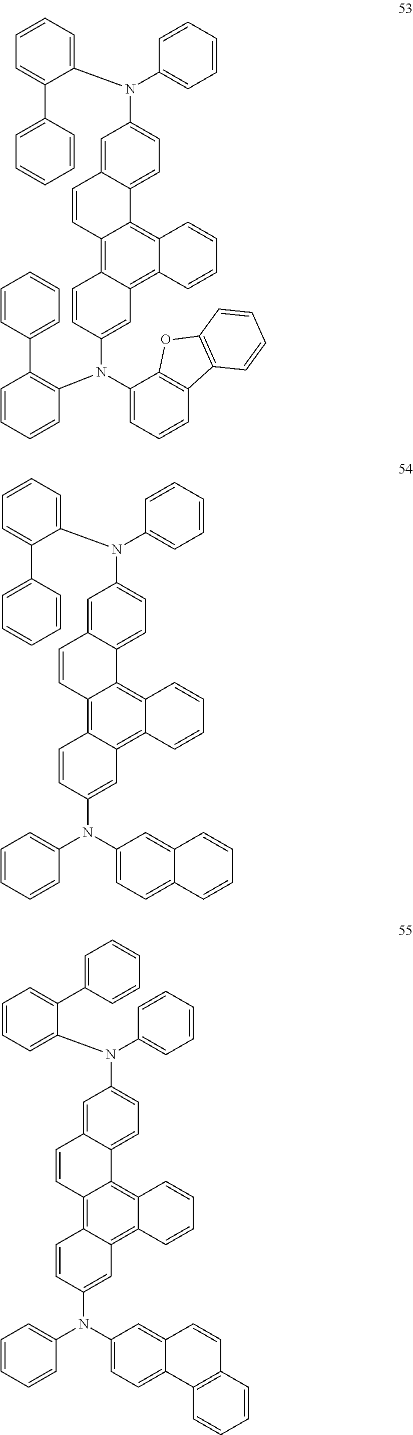

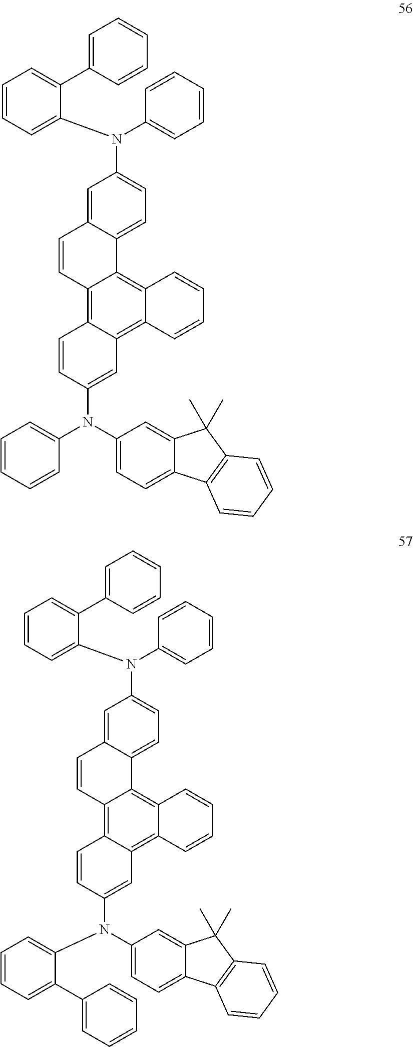 [SMILES: CC1(C)c2ccccc2-c2ccc(N(c3ccccc3)c3ccc4c(c3)c3ccccc3c3c5ccc(N(c6ccccc6)c6ccccc6-c6ccccc6)cc5ccc43)cc21 ; CC1(C)c2ccccc2-c2ccc(N(c3ccc4c(c3)c3ccccc3c3c5ccc(N(c6ccccc6)c6ccccc6-c6ccccc6)cc5ccc43)c3ccccc3-c3ccccc3)cc21]

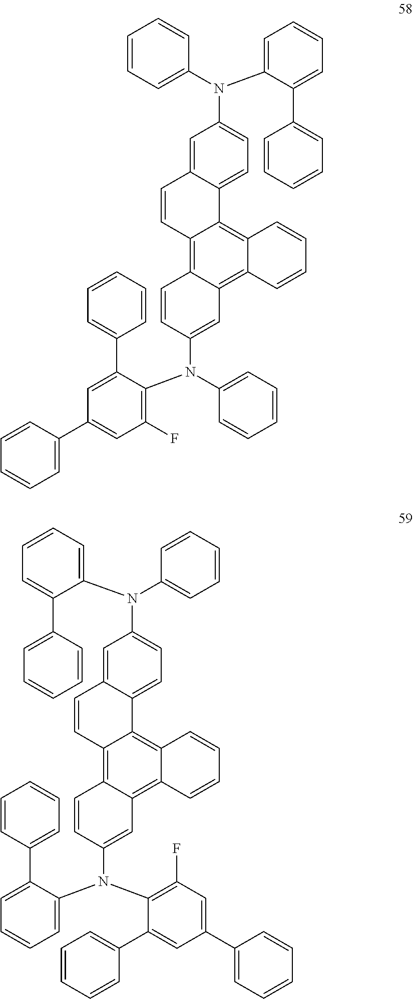

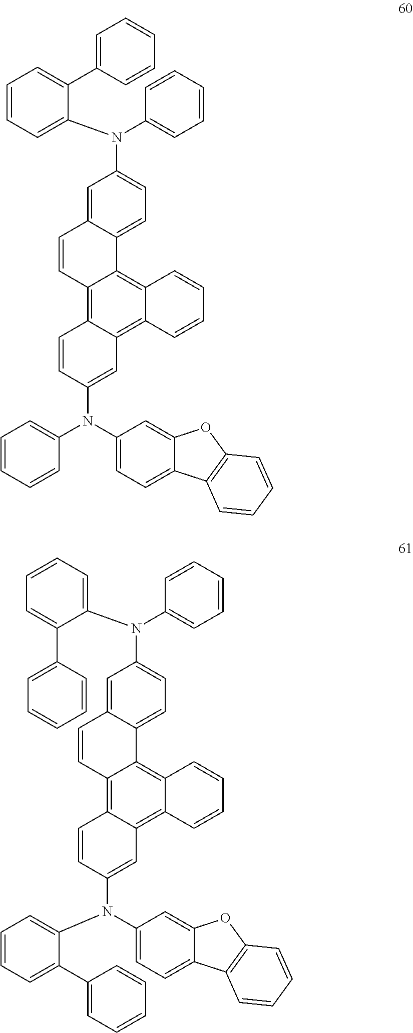 [SMILES: c1ccc(-c2ccccc2N(c2ccccc2)c2ccc3c(ccc4c5ccc(N(c6ccccc6)c6ccc7c(c6)oc6ccccc67)cc5c5ccccc5c34)c2)cc1 ; c1ccc(-c2ccccc2N(c2ccccc2)c2ccc3c(ccc4c5ccc(N(c6ccc7c(c6)oc6ccccc67)c6ccccc6-c6ccccc6)cc5c5ccccc5c34)c2)cc1]

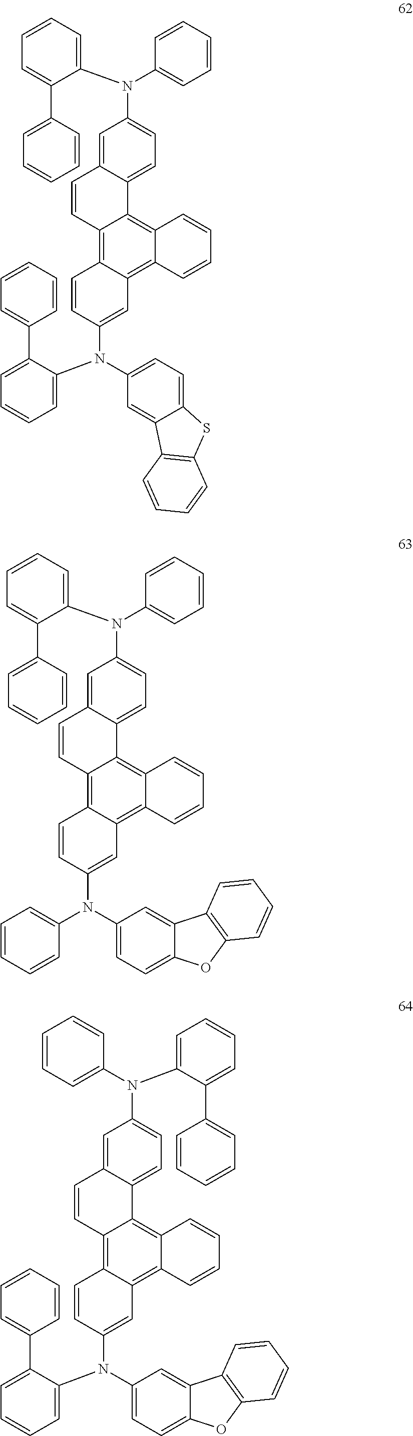

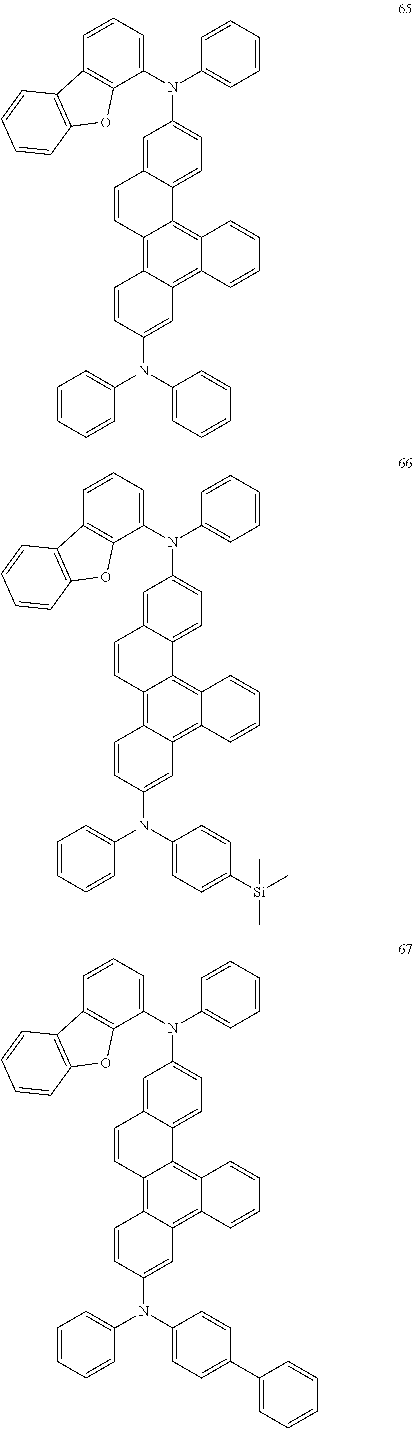

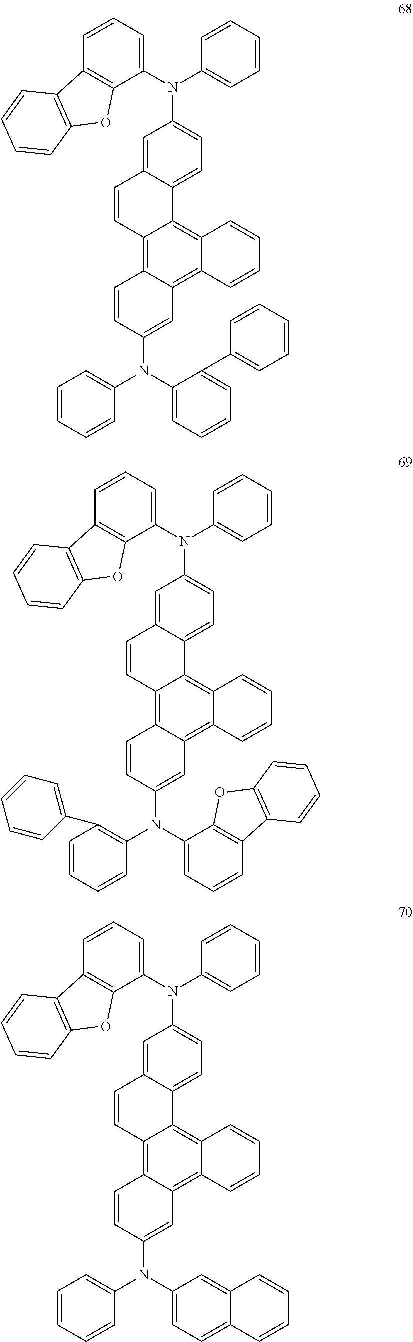

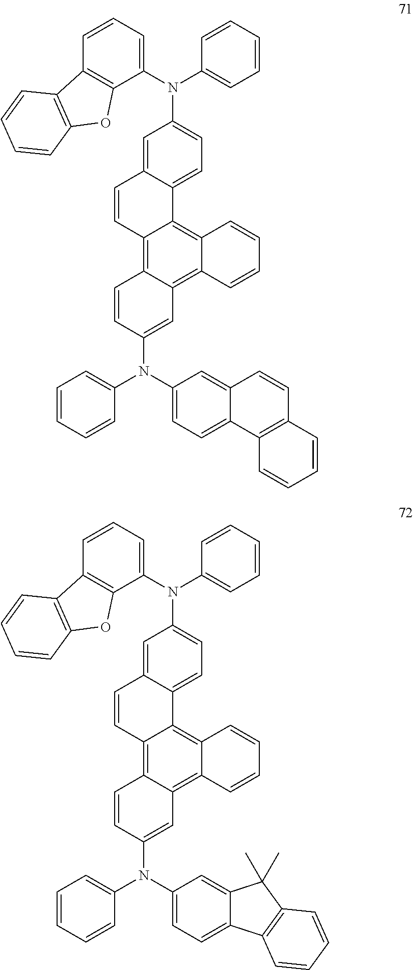 [SMILES: c1ccc(N(c2ccc3c(ccc4ccccc43)c2)c2ccc3c(c2)c2ccccc2c2c4ccc(N(c5ccccc5)c5cccc6c5oc5ccccc56)cc4ccc32)cc1 ; CC1(C)c2ccccc2-c2ccc(N(c3ccccc3)c3ccc4c(c3)c3ccccc3c3c5ccc(N(c6ccccc6)c6cccc7c6oc6ccccc67)cc5ccc43)cc21]

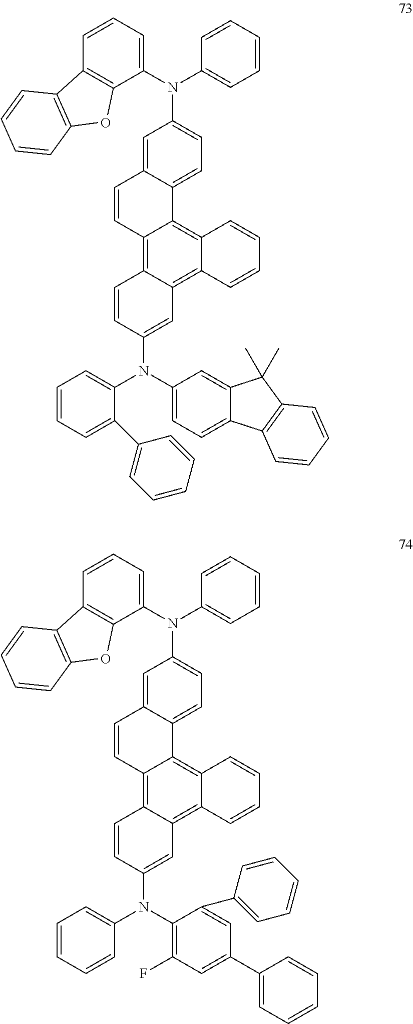 [SMILES: CC1(C)c2ccccc2-c2ccc(N(c3ccc4c(c3)c3ccccc3c3c5ccc(N(c6ccccc6)c6cccc7c6oc6ccccc67)cc5ccc43)c3ccccc3-c3ccccc3)cc21 ; Fc1cc(-c2ccccc2)cc(-c2ccccc2)c1N(c1ccccc1)c1ccc2c(c1)c1ccccc1c1c3ccc(N(c4ccccc4)c4cccc5c4oc4ccccc45)cc3ccc21]

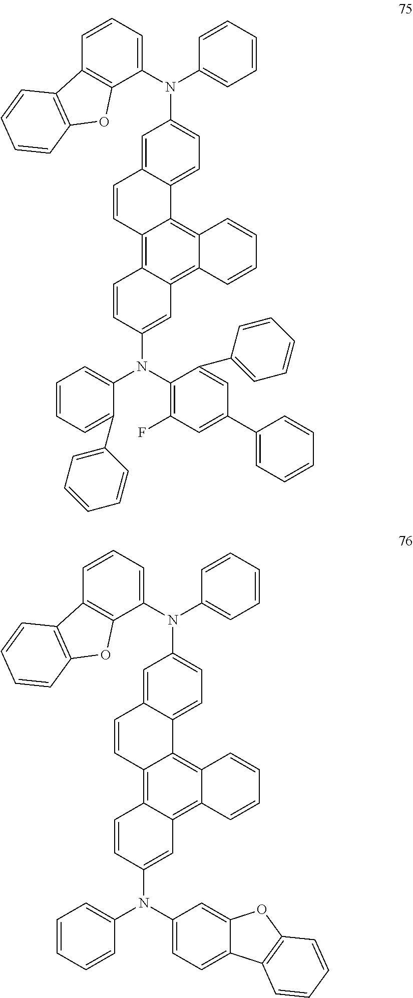

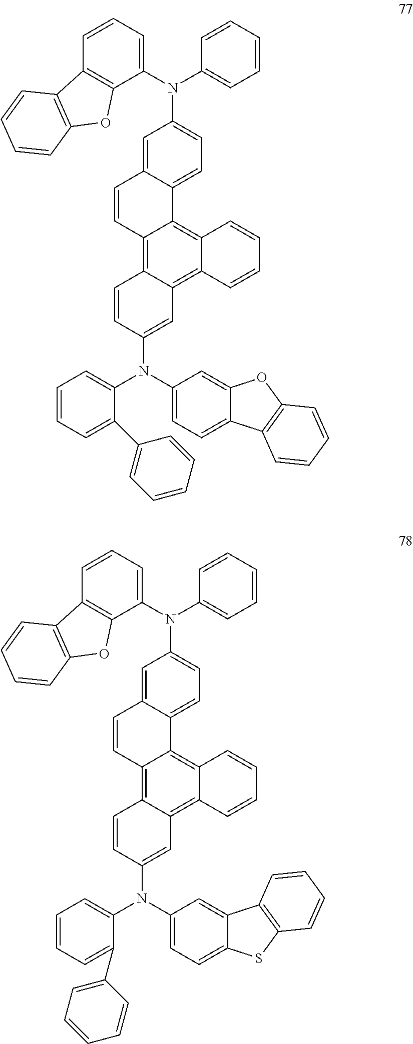 [SMILES: c1ccc(-c2ccccc2N(c2ccc3c(c2)oc2ccccc23)c2ccc3c(c2)c2ccccc2c2c4ccc(N(c5ccccc5)c5cccc6c5oc5ccccc56)cc4ccc32)cc1 ; c1ccc(-c2ccccc2N(c2ccc3sc4ccccc4c3c2)c2ccc3c(c2)c2ccccc2c2c4ccc(N(c5ccccc5)c5cccc6c5oc5ccccc56)cc4ccc32)cc1]

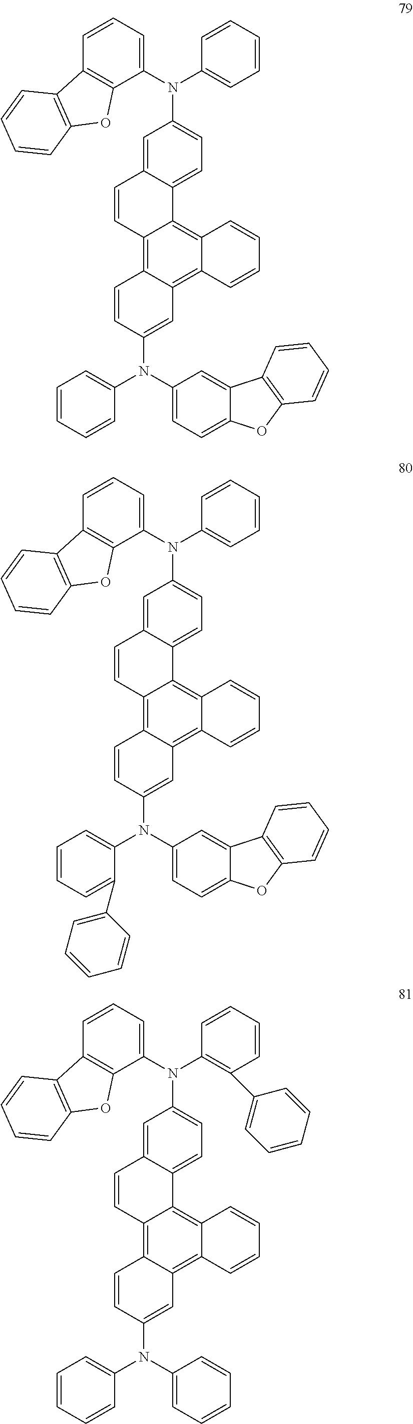

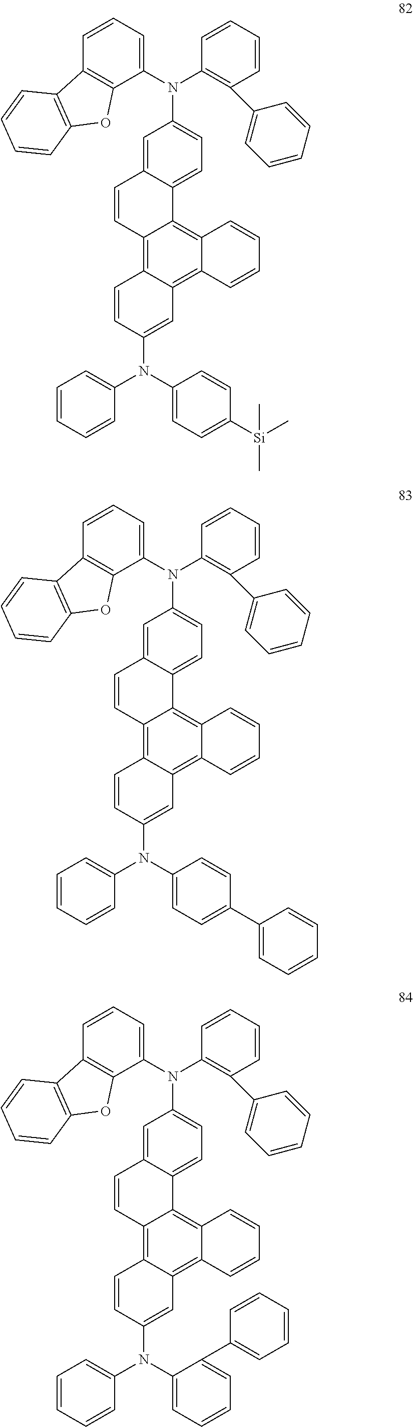 [SMILES: C[Si](C)(C)c1ccc(N(c2ccccc2)c2ccc3c(c2)c2ccccc2c2c4ccc(N(c5ccccc5-c5ccccc5)c5cccc6c5oc5ccccc56)cc4ccc32)cc1 ; c1ccc(-c2ccc(N(c3ccccc3)c3ccc4c(c3)c3ccccc3c3c5ccc(N(c6ccccc6-c6ccccc6)c6cccc7c6oc6ccccc67)cc5ccc43)cc2)cc1 ; c1ccc(-c2ccccc2N(c2ccccc2)c2ccc3c(c2)c2ccccc2c2c4ccc(N(c5ccccc5-c5ccccc5)c5cccc6c5oc5ccccc56)cc4ccc32)cc1]

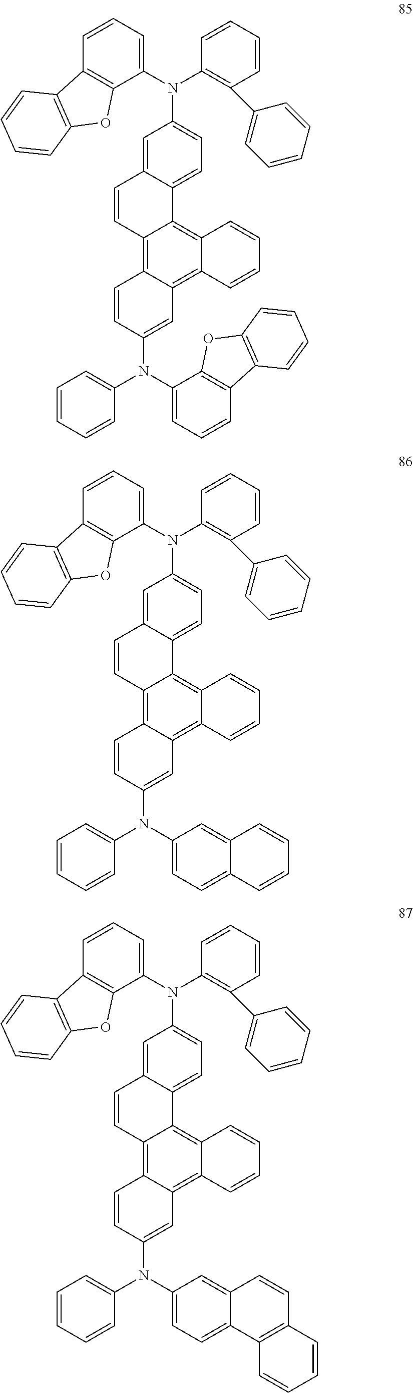

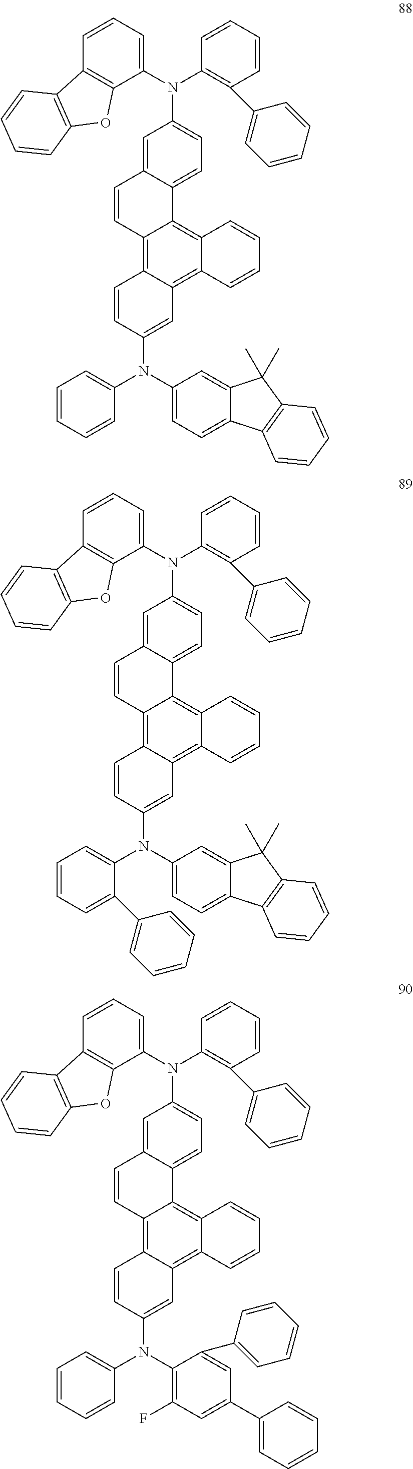

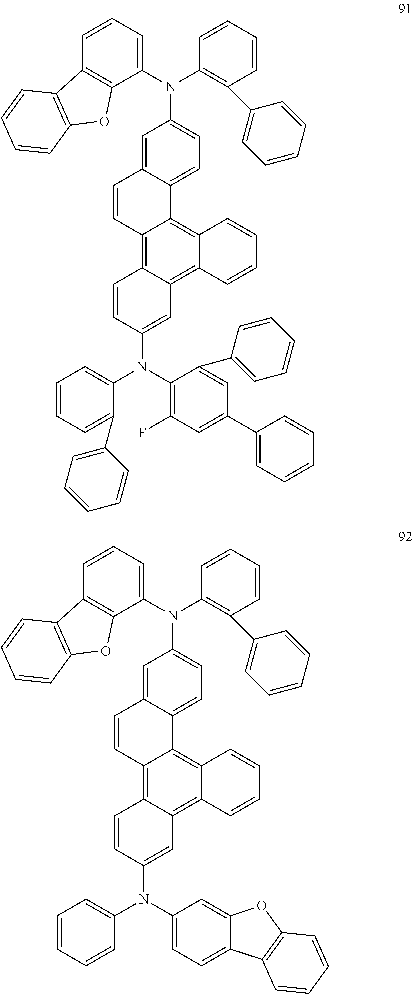

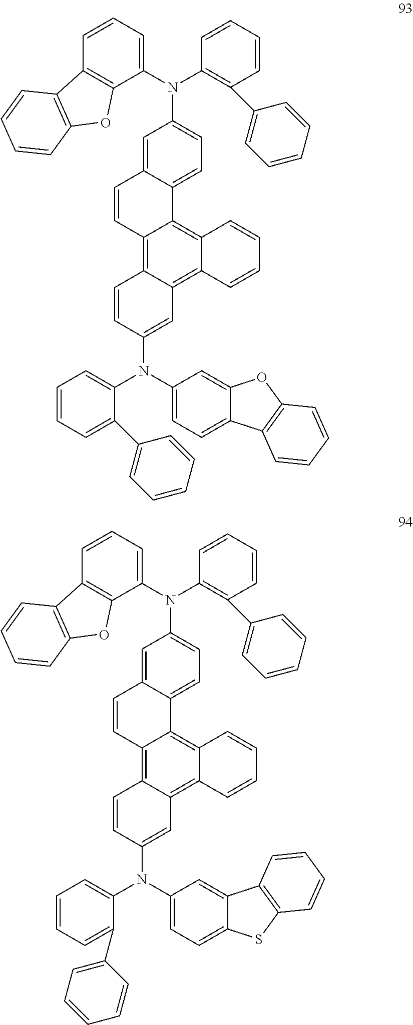 [SMILES: c1ccc(-c2ccccc2N(c2ccc3c(c2)oc2ccccc23)c2ccc3c(c2)c2ccccc2c2c4ccc(N(c5ccccc5-c5ccccc5)c5cccc6c5oc5ccccc56)cc4ccc32)cc1 ; c1ccc(-c2ccccc2N(c2ccc3sc4ccccc4c3c2)c2ccc3c(c2)c2ccccc2c2c4ccc(N(c5ccccc5-c5ccccc5)c5cccc6c5oc5ccccc56)cc4ccc32)cc1]

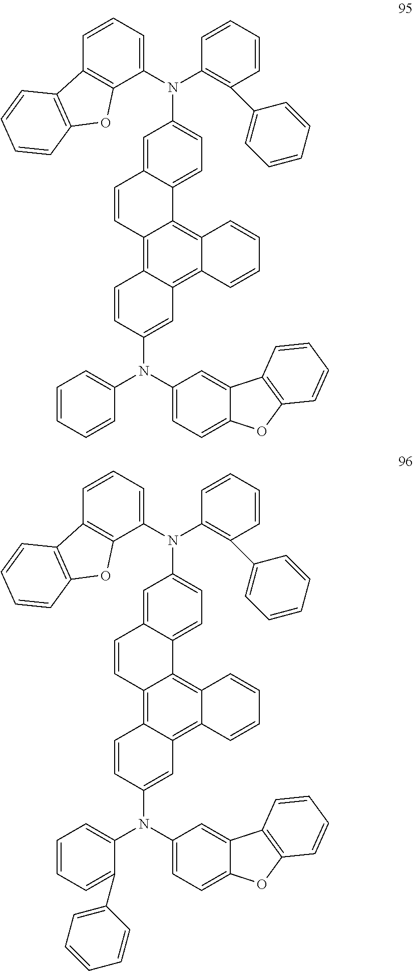

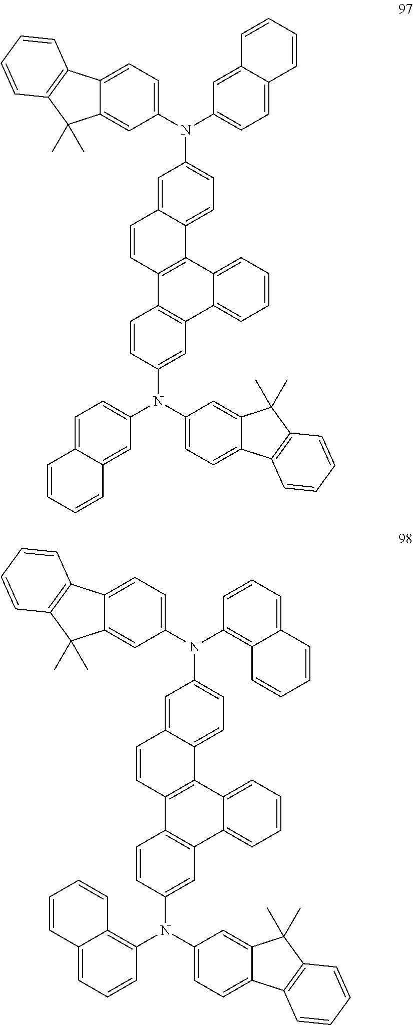

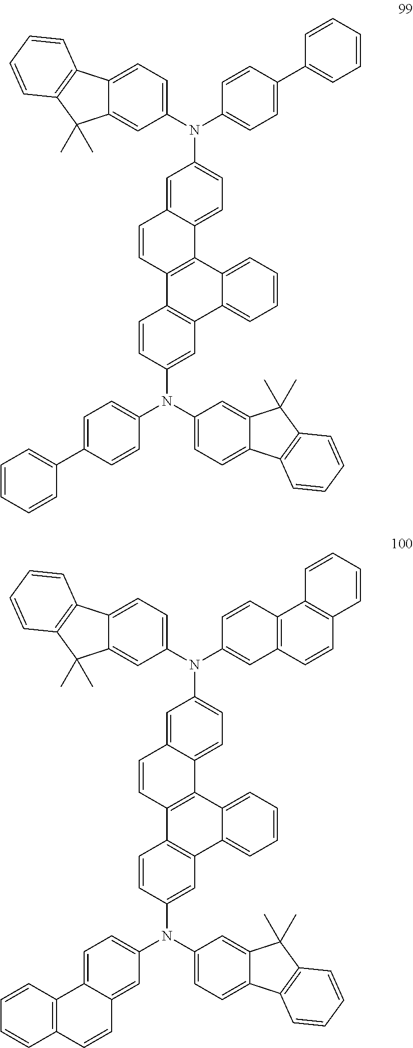 [SMILES: CC1(C)c2ccccc2-c2ccc(N(c3ccc(-c4ccccc4)cc3)c3ccc4c(ccc5c6ccc(N(c7ccc(-c8ccccc8)cc7)c7ccc8c(c7)C(C)(C)c7ccccc7-8)cc6c6ccccc6c45)c3)cc21 ; CC1(C)c2ccccc2-c2ccc(N(c3ccc4c(ccc5ccccc54)c3)c3ccc4c(ccc5c6ccc(N(c7ccc8c(c7)C(C)(C)c7ccccc7-8)c7ccc8c(ccc9ccccc98)c7)cc6c6ccccc6c45)c3)cc21]

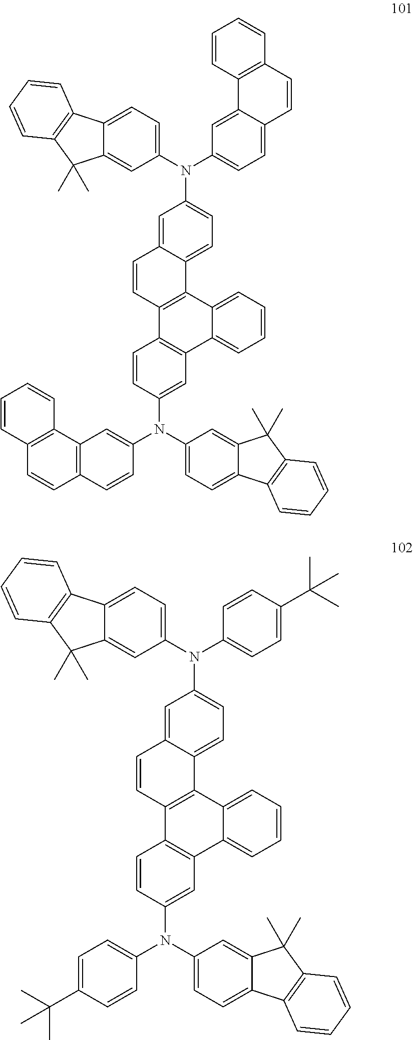

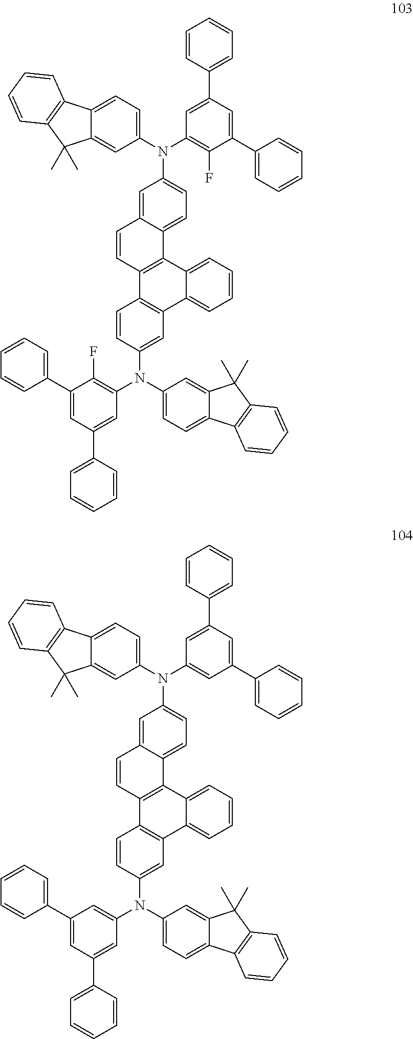 [SMILES: CC1(C)c2ccccc2-c2ccc(N(c3ccc4c(ccc5c6ccc(N(c7ccc8c(c7)C(C)(C)c7ccccc7-8)c7cc(-c8ccccc8)cc(-c8ccccc8)c7F)cc6c6ccccc6c45)c3)c3cc(-c4ccccc4)cc(-c4ccccc4)c3F)cc21 ; CC1(C)c2ccccc2-c2ccc(N(c3cc(-c4ccccc4)cc(-c4ccccc4)c3)c3ccc4c(ccc5c6ccc(N(c7cc(-c8ccccc8)cc(-c8ccccc8)c7)c7ccc8c(c7)C(C)(C)c7ccccc7-8)cc6c6ccccc6c45)c3)cc21]

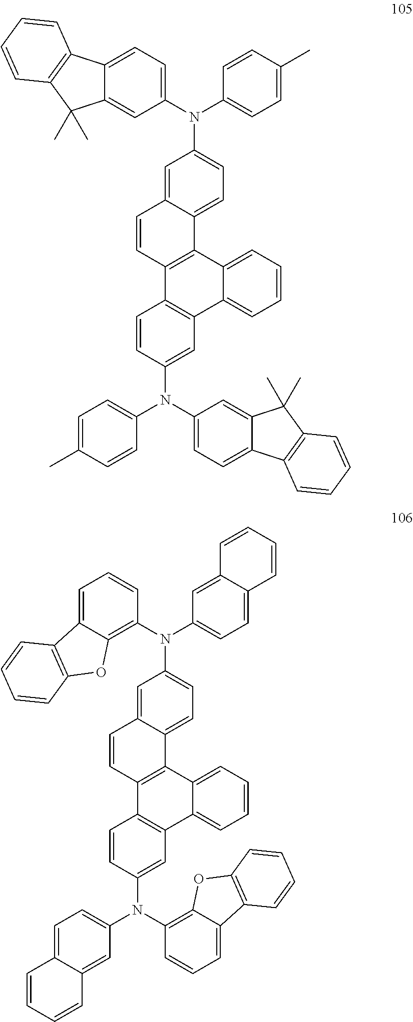 [SMILES: Cc1ccc(N(c2ccc3c(c2)C(C)(C)c2ccccc2-3)c2ccc3c(ccc4c5ccc(N(c6ccc(C)cc6)c6ccc7c(c6)C(C)(C)c6ccccc6-7)cc5c5ccccc5c34)c2)cc1 ; c1ccc2cc(N(c3ccc4c(ccc5c6ccc(N(c7ccc8ccccc8c7)c7cccc8c7oc7ccccc78)cc6c6ccccc6c45)c3)c3cccc4c3oc3ccccc34)ccc2c1]

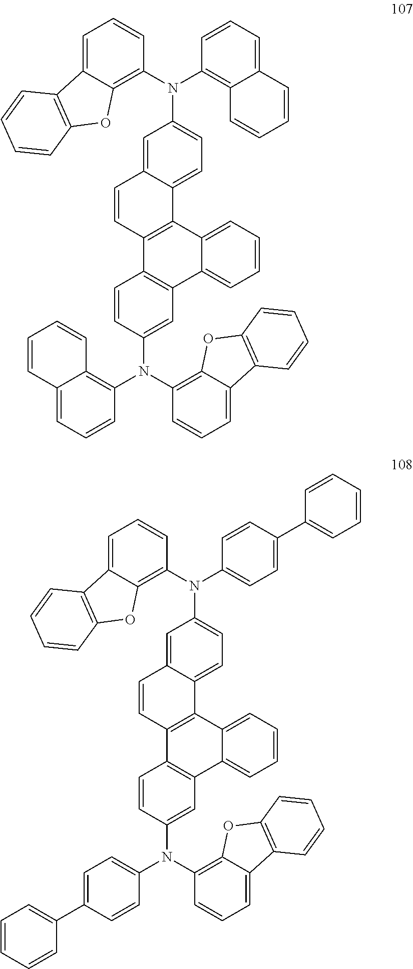 [SMILES: c1ccc2c(N(c3ccc4c(ccc5c6ccc(N(c7cccc8ccccc78)c7cccc8c7oc7ccccc78)cc6c6ccccc6c45)c3)c3cccc4c3oc3ccccc34)cccc2c1 ; c1ccc(-c2ccc(N(c3ccc4c(ccc5c6ccc(N(c7ccc(-c8ccccc8)cc7)c7cccc8c7oc7ccccc78)cc6c6ccccc6c45)c3)c3cccc4c3oc3ccccc34)cc2)cc1]

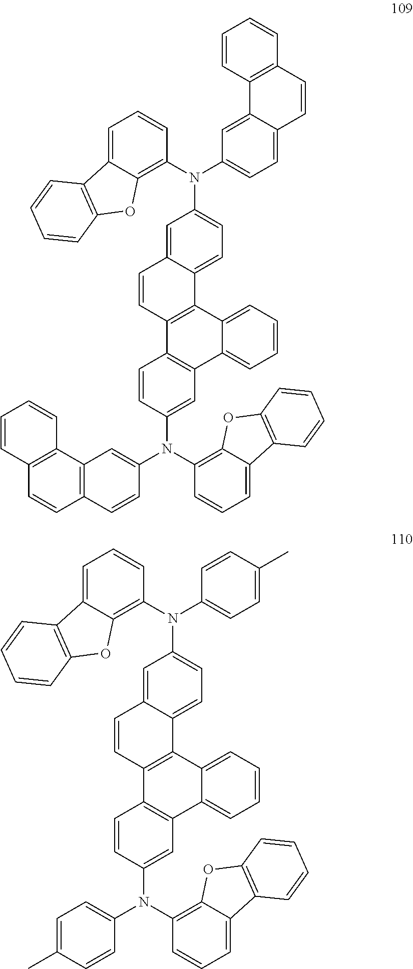

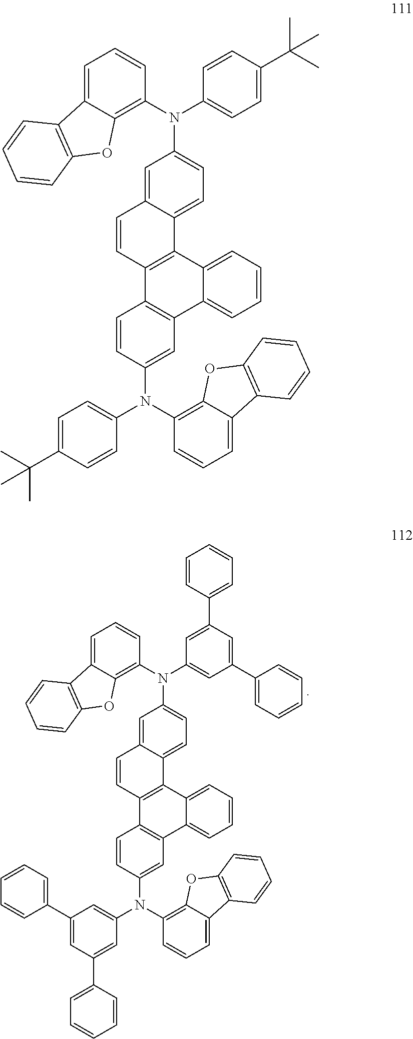 [SMILES: CC(C)(C)c1ccc(N(c2ccc3c(ccc4c5ccc(N(c6ccc(C(C)(C)C)cc6)c6cccc7c6oc6ccccc67)cc5c5ccccc5c34)c2)c2cccc3c2oc2ccccc23)cc1 ; c1ccc(-c2cc(-c3ccccc3)cc(N(c3ccc4c(ccc5c6ccc(N(c7cc(-c8ccccc8)cc(-c8ccccc8)c7)c7cccc8c7oc7ccccc78)cc6c6ccccc6c45)c3)c3cccc4c3oc3ccccc34)c2)cc1]

The expression “organic layer” used herein refers to a single layer and/or a plurality of layers between the first electrode and the second electrode of the organic light-emitting device. A material included in the “organic layer” is not limited to an organic material.

The drawing illustrates a schematic view of an organic light-emitting device 10 according to one or more embodiments of the present invention. The organic light-emitting device 10 may include a first electrode 110, an organic layer 150, and a second electrode 190.

Hereinafter, a structure of the organic light-emitting device 10 according to one or more embodiments of the present invention and a method of manufacturing the organic light-emitting device 10 will be described in connection with the drawing.

In the drawing, a substrate may be positioned under the first electrode 110 or on the second electrode 190. The substrate may be a glass substrate or a transparent plastic substrate, each with excellent mechanical strength, thermal stability, transparency, surface smoothness, ease of handling, and/or water repellency (or resistance).

The first electrode 110 may be formed by, for example, depositing or sputtering a material for forming the first electrode 110 on the substrate. When the first electrode 110 is an anode, the material for forming the first electrode 110 may be selected from materials with a high work function so as to facilitate hole injection. The first electrode 110 may be, for example, a reflective electrode, a semi-transparent electrode, or a transparent electrode. The material for forming the first electrode 110 may be a transparent and highly conductive material, and non-limiting examples thereof include tin indium (ITO), indium zinc oxide (IZO), tin oxide (SnO₂), and zinc oxide (ZnO). In some embodiments, when the first electrode 110 is a semi-transparent electrode or a reflective electrode, the material for forming the first electrode 110 may include at least one selected from magnesium (Mg), aluminum (Al), aluminum-lithium (Al—Li), calcium (Ca), magnesium-indium (Mg—In), and magnesium-silver (Mg—Ag).

The first electrode 110 may have a single-layered structure or a multi-layered structure including a plurality of layers. For example, the first electrode 110 may have a three-layered structure of ITO/Ag/ITO, but the structure is not limited thereto.

The organic layer 150 may be positioned on the first electrode 110. The organic layer 150 may include an emission layer.

The organic layer 150 may further include a hole transport region between the first electrode 110 and the emission layer and an electron transport region between the emission layer and the second electrode 190.

The hole transport region may include at least one selected from a hole transport layer (HTL), a hole injection layer (HIL), a buffer layer, and an electron blocking layer (EBL), and the electron transport region may include at least one selected from a hole blocking layer (HBL), an electron transport layer (ETL), and an electron injection layer (EIL), but the structures of the hole transport region and the electron transport region are not limited thereto.

The hole transport region may have a single-layered structure formed of a single material, a single-layered structure formed of a plurality of different materials, or a multi-layered structure having a plurality of layers formed of different materials.

For example, the hole transport region may have a single-layered structure formed of different materials, or a structure of HIL/HTL, a structure of HIL/HTL/buffer layer, a structure of HIL/buffer layer, a structure of HTL/buffer layer, or a structure of HIL/HTL/EBL, where the layers of each structure are sequentially stacked on the first electrode 110 in the stated order, but the structure is not limited thereto.

When hole transport region includes a HIL, the HIL may be formed on the first electrode 110 by using (utilizing) one or more suitable methods, such as vacuum deposition, spin coating, casting, a Langmuir-Blodgett (LB) method, an ink-jet printing, a laser-printing, and/or a laser induced thermal imaging (LITI) method.

When the HIL is formed by vacuum deposition, the deposition may be performed, for example, at a deposition temperature of about 100° C. to about 500° C., at a vacuum degree of about 10⁻⁸ torr to about 10⁻³ torr, and a deposition rate of about 0.01 Å/sec to about 100 Å/sec depending on the compound for forming the HIL and a structure of the HIL to be formed.

When the HIL is formed by spin coating, the coating may be performed, for example, at a coating speed of about 2,000 rpm to about 5,000 rpm and at a temperature of about 80° C. to about 200° C., depending on the compound for forming the HIL and a structure of the HIL to be formed.

When the hole transport region includes a HTL, the HTL may be formed on the first electrode 110 or on the HIL by using one or more suitable methods, such as vacuum deposition, spin coating, casting, an LB method, an ink-jet printing, a laser-printing, and/or an LITI method. When the HTL is formed by vacuum deposition and/or spin coating, the deposition and coating conditions for the HTL may be similar to the deposition and coating conditions for the HIL.

The hole transport region may include at least one selected from m-MTDATA, TDATA, 2-TNATA, NPB, β-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzenesulfonic acid (Pani/DBSA), poly(3,4-ethylenedioxythiophene)/poly(4-styrenesulfonate) (PEDOT/PSS), polyaniline/camphor sulfonicacid (Pani/CSA), polyaniline/poly(4-styrenesulfonate (PANI/PSS), a compound represented by Formula 201, and a compound represented by Formula 202, below:

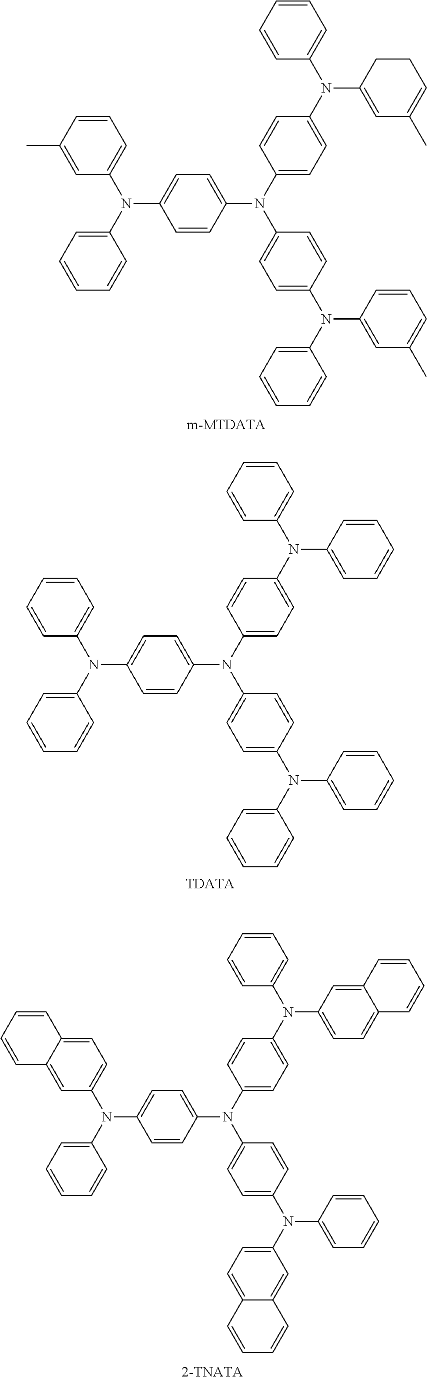

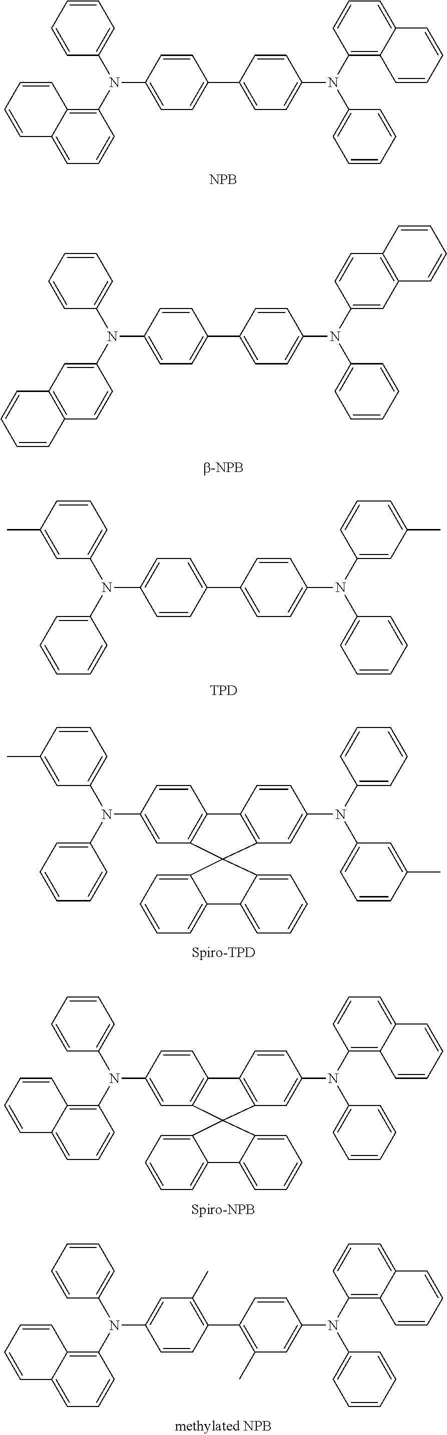

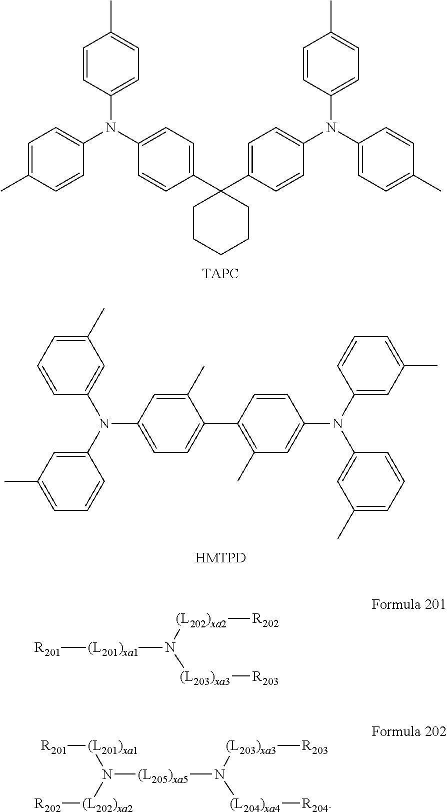

In Formulae 201 and 202,

L₂₀₁ to L₂₀₅ may be each independently understood by referring to the description provided in connection with L₁;

xa1 to xa4 may each independently be 0, 1, 2, or 3;

xa5 may be 1, 2, 3, 4, or 5; and

R₂₀₁ to R₂₀₄ may each independently be selected from a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

For example, in Formulae 201 and 202,

L₂₀₁ to L₂₀₅ may each independently be selected from:

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, an isoindolyl group, a quinolinyl group, an isoquinolinyl group, a quinoxalinyl group, a quinazolinyl group, a carbazolyl group, and a triazinyl group;

xa1 to xa4 may each independently be selected from 0, 1, and 2;

xa5 may be selected from 1, 2, and 3;

R₂₀₁ to R₂₀₄ may each independently be selected from a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, an azulenyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, but they are not limited thereto.

The compound of Formula 201 may be represented by Formula 201A below:

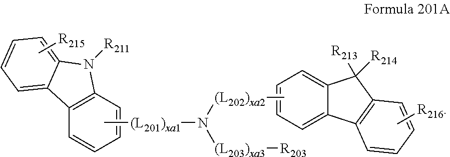

For example, the compound of Formula 201 may be represented by Formula 201A-1 below, but is not limited thereto:

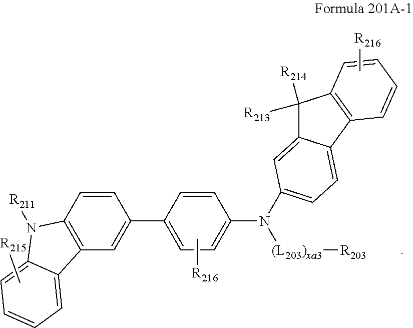

The compound of Formula 202 may be represented by Formula 202A below, but is not limited thereto:

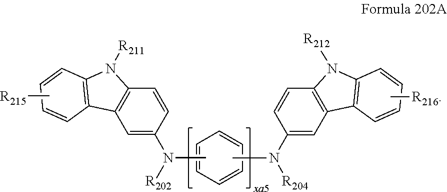

In Formulae 201A, 201A-1, and 202A, L₂₀₁ to L₂₀₃, xa1 to xa3, xa5, and R₂₀₂ to R₂₀₄ may be understood by referring to the descriptions provided herein; R₂₁₁ may be understood by referring to the description provided in connection with R₂₀₃; and R₂₁₃ to R₂₁₆ may each independently be selected from hydrogen, deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

For example, in Formulae 201A, 201A-1, and 202A,

L₂₀₁ to L₂₀₃ may each independently be selected from:

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

xa1 to xa3 may each independently be 0 or 1;

R₂₀₃, R₂₁₁, and R₂₁₂ may each independently be selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —CI, —Br, —I, a hydroxyl group, a cyano group, a nitro group, an amino group, an amidino group, a hydrazine group, a hydrazone group, carboxylic acid a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

R₂₁₃ and R₂₁₄ may each independently be selected from:

a C₁-C₂₀ alkyl group and a C₁-C₂₀ alkoxy group;

a C₁-C₂₀ alkyl group and a C₁-C₂₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

R₂₁₅ and R₂₁₆ may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

xa5 may be 1 or 2.

In Formulae 201A and 201A-1, R₂₁₃ and R₂₁₄ may be bound to each other to form a saturated or unsaturated ring.

The compound of Formula 201 and the compound of Formula 202 may each independently include Compounds HT1 to HT20 below, but are not limited thereto.

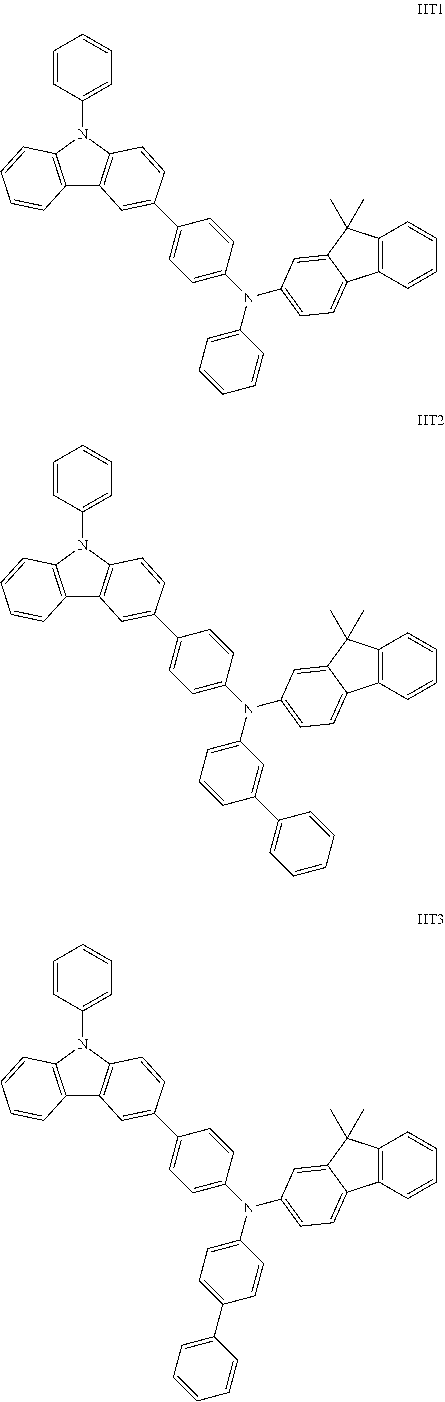 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

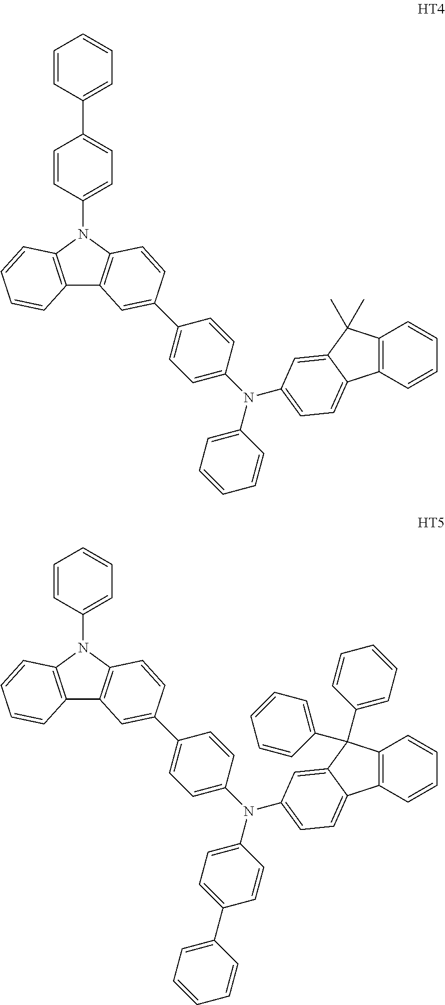 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

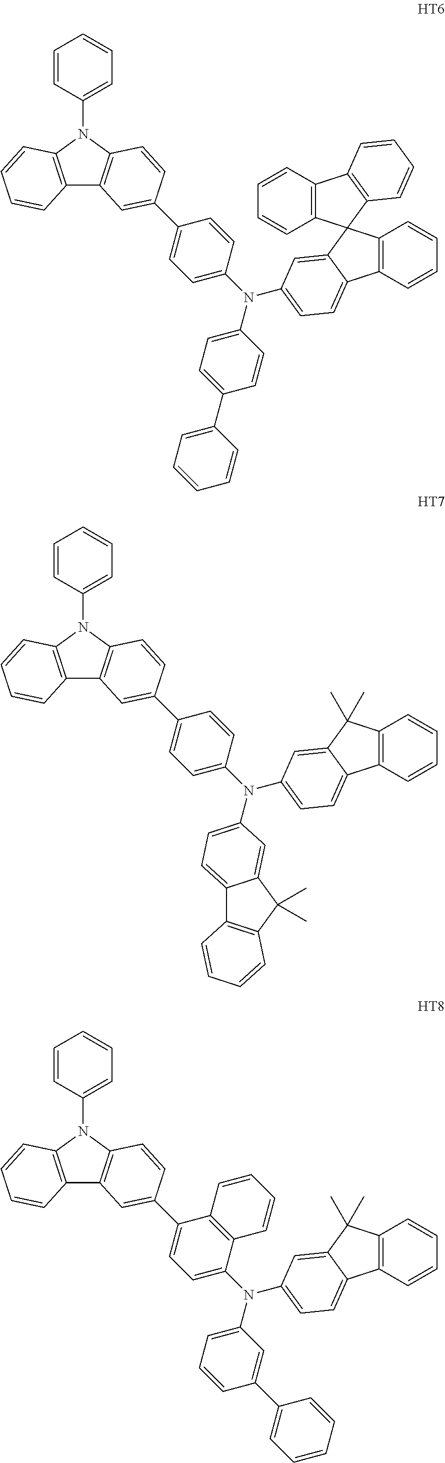 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21]

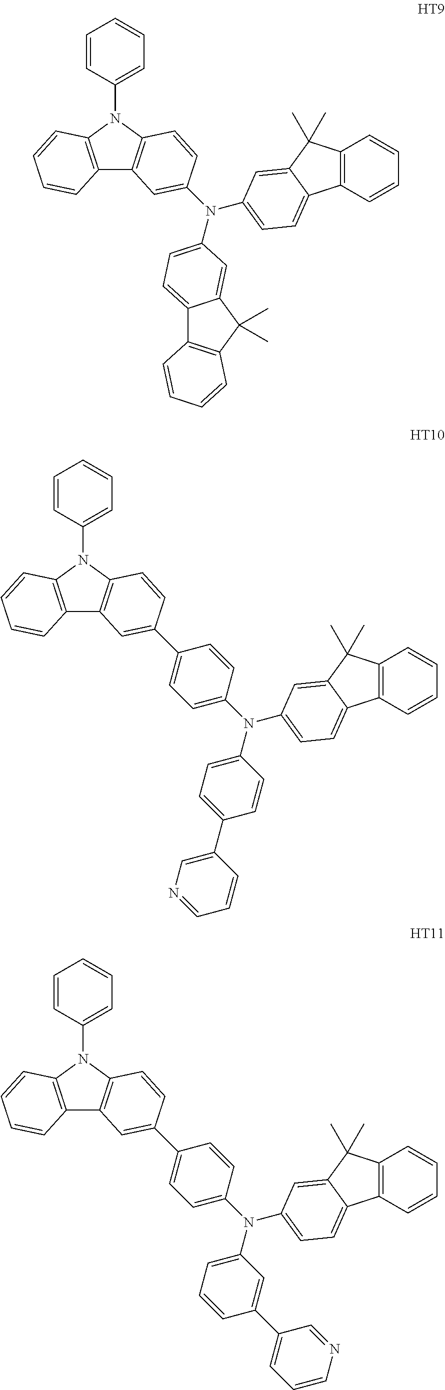 [SMILES: CC1(C)c2ccccc2-c2ccc(N(c3ccc4c(c3)C(C)(C)c3ccccc3-4)c3ccc4c(c3)c3ccccc3n4-c3ccccc3)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21]

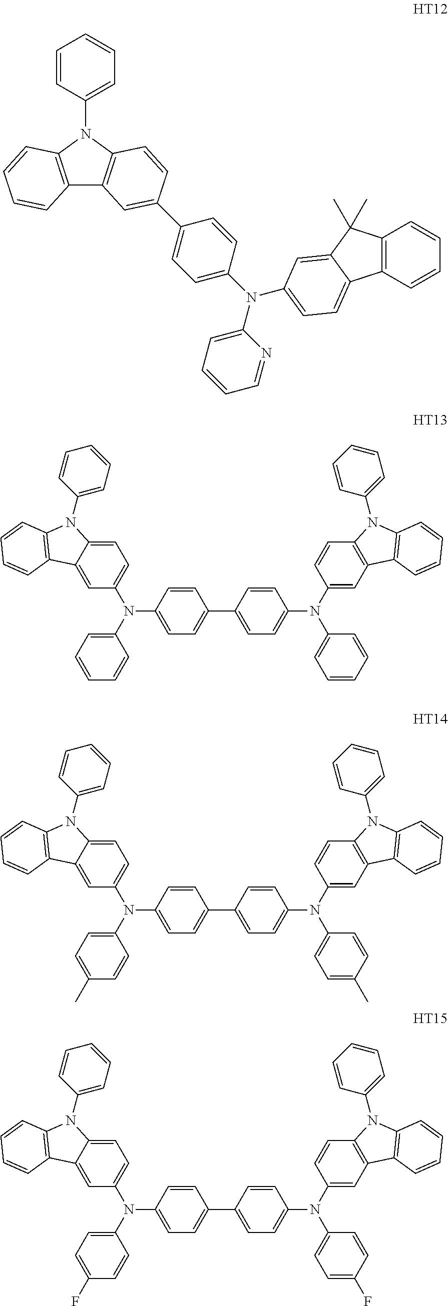 [SMILES: CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

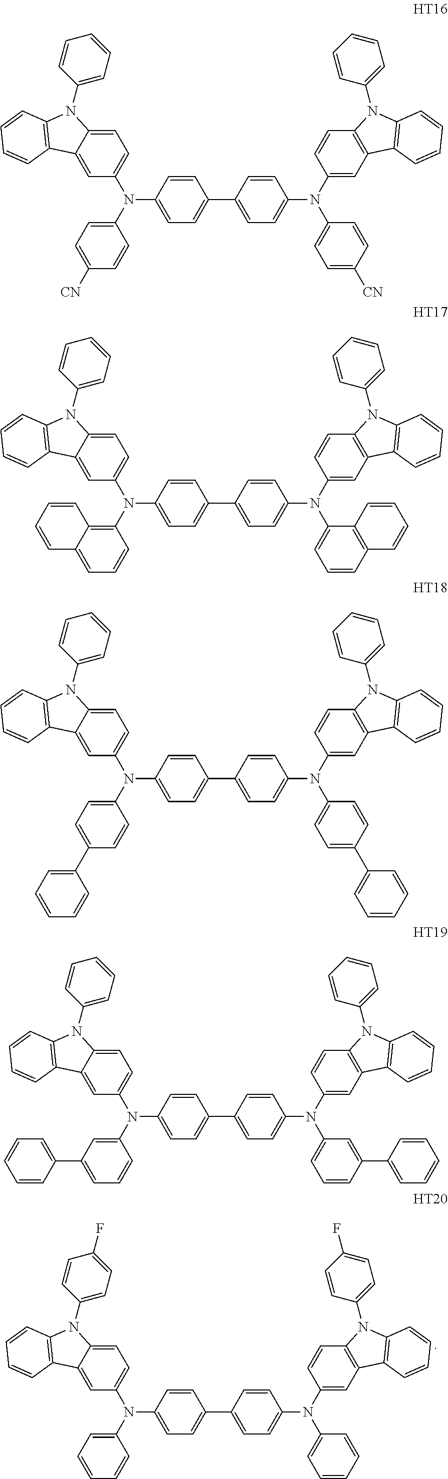

A thickness of the hole transport region may be about 100 Å to about 10,000 Å, for example, about 100 Å to about 1,000 Å. When the hole transport region includes both an HIL and an HTL, a thickness of the HIL may be about 100 Å to about 10,000 Å, for example, about 100 Å to about 9,950 Å, or about 100 Å to about 1,000 Å, and a thickness of the HTL may be about 50 Å to about 2,000 Å, for example, about 100 Å to about 1,500 Å. When the thicknesses of the hole transport region, the HIL, and the HTL are within any of these ranges, satisfactory hole transport characteristics may be obtained without a substantial increase in driving voltage.

The hole transport region may further include, in addition to the above-described materials, a charge-generation material for the improvement of conductivity. The charge-generation material may be homogeneously or non-homogeneously dispersed in the hole transport region.

The charge-generation material may be, for example, a p-dopant. The p-dopant may include, for example, at least one selected from a quinone derivative, a metal oxide, and a cyano group-containing compound, but is not limited thereto. Non-limiting examples of the p-dopant may include quinone derivatives, such as tetracyanoquinonedimethane (TCNQ) and/or 2,3,5,6-tetrafluoro-tetracyano-1,4-benzoquinonedimethane (F4-TCNQ); metal oxides, such as tungsten oxide and/or molybdenum oxide; and Compound HT-D1 below.

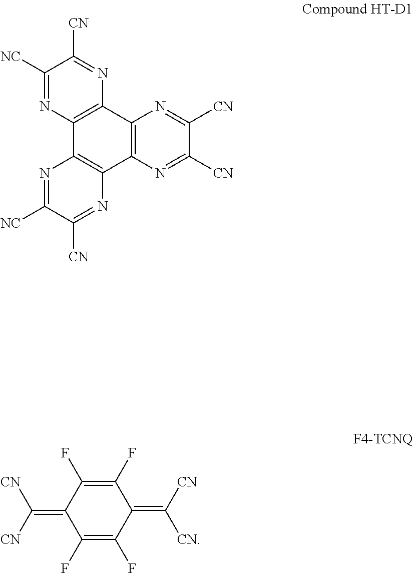

The hole transport region may further include, in addition to the HIL and the HTL, a buffer layer and/or a EBL. The buffer layer may help compensate for an optical resonance distance according to a wavelength of light emitted from the emission layer, and thus, a light-emission efficiency of the formed organic light-emitting device may be improved. For use as a material for forming the buffer layer, one or more materials for forming the hole transport region may be used (utilized). The EBL may prevent (or substantially block) electron injection from the electron transport region.

The emission layer may be formed on the first electrode 110 or on the hole transport region by using one or more suitable methods, such as vacuum deposition, spin coating, casting, an LB method, an ink-jet printing, a laser-printing, and/or an LITI method. When the emission layer is formed by vacuum deposition and/or spin coating, the deposition and coating conditions for forming the emission layer may be similar to the deposition and coating conditions for forming the HIL.

When the organic light-emitting device 10 is a full color organic light-emitting device, the emission layer may be patterned into a red emission layer, a green emission layer, and a blue emission layer, according to a sub-pixel. In some embodiments, the emission layer may have a stacked structure of a red emission layer, a green emission layer, and a blue emission layer, or may include a red light emission material, a green light emission material, and a blue light emission material, which are mixed with each other in a single layer, to emit white light.

The emission layer may include a host and a dopant.

The host may include, for example, at least one selected from TPBi, TBADN, AND (also known as “DNA” or “ADN”), CBP, CDBP, and TCP below:

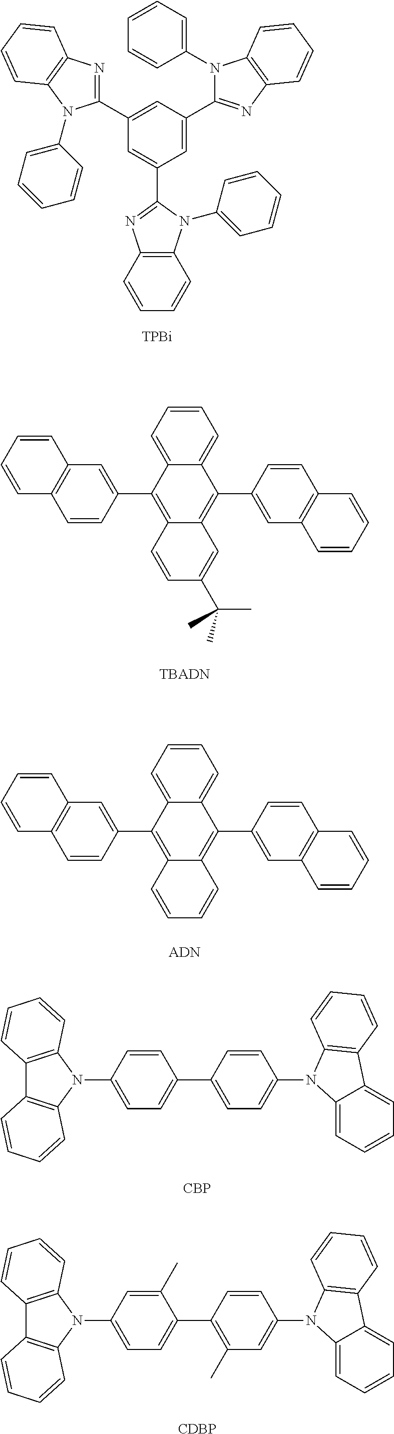

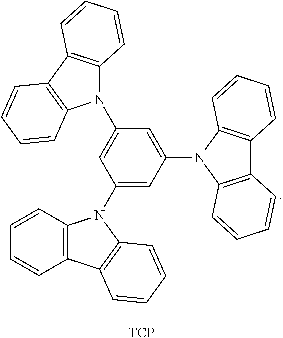

In some embodiments, the host may include a compound represented by Formula 301 below:

Ar₃₀₀-[(L₃₀₁)_{xb1}-R₃₀₁]_{xb2}. Formula 301

In Formula 301,

Ar₃₀₁ may be selected from:

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene; and

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene, each substituted with at least one selected from deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₃₀₁)(Q₃₀₂)(Q₃₀₃) (where Q₃₀₁ to Q₃₀₃ may each independently be selected from hydrogen, a C₁-C₆₀ alkyl group, a C₂-C₆₀ alkenyl group, a C₆-C₆₀ aryl group, and a C₁-C₆₀ heteroaryl group);

L₃₀₁ may be understood by referring to the description provided in connection with L₂₀₁;

R₃₀₁ may be selected from:

a C₁-C₂₀ alkyl group and a C₁-C₂₀ alkoxy group;

a C₁-C₂₀ alkyl group and a C₁-C₂₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

xb1 may be selected from 0, 1, 2, and 3; and

xb2 may be selected from 1, 2, 3, and 4.

For example, in Formula 301,

L₃₀₁ may be selected from:

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, and a chrysenylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, and a chrysenylene group, each substituted with at least one selected from deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group; and

R₃₀₁ may be selected from:

a C₁-C₂₀ alkyl group and a C₁-C₂₀ alkoxy group;

a C₁-C₂₀ alkyl group and a C₁-C₂₀ alkoxy group, each substituted with at least one selected from deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group, each substituted with at least one selected from deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group; but is not limited thereto.

For example, the host may include a compound represented by Formula 301A below:

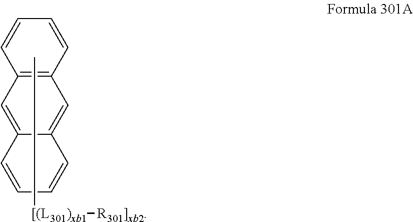

Definitions of the substituents used in Formula 301A are as described above.

The compound of Formula 301 may include at least one selected from Compounds H1 to H42 below, but is not limited thereto.

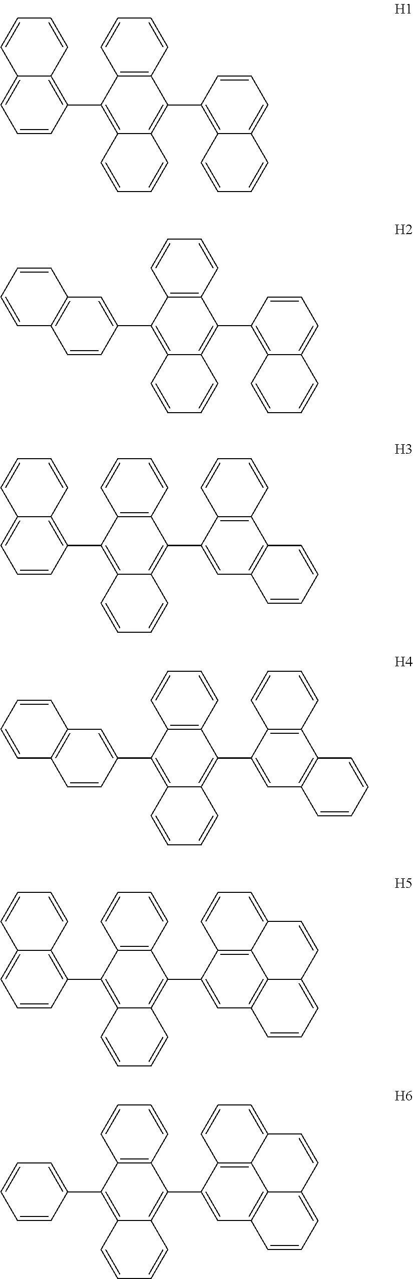

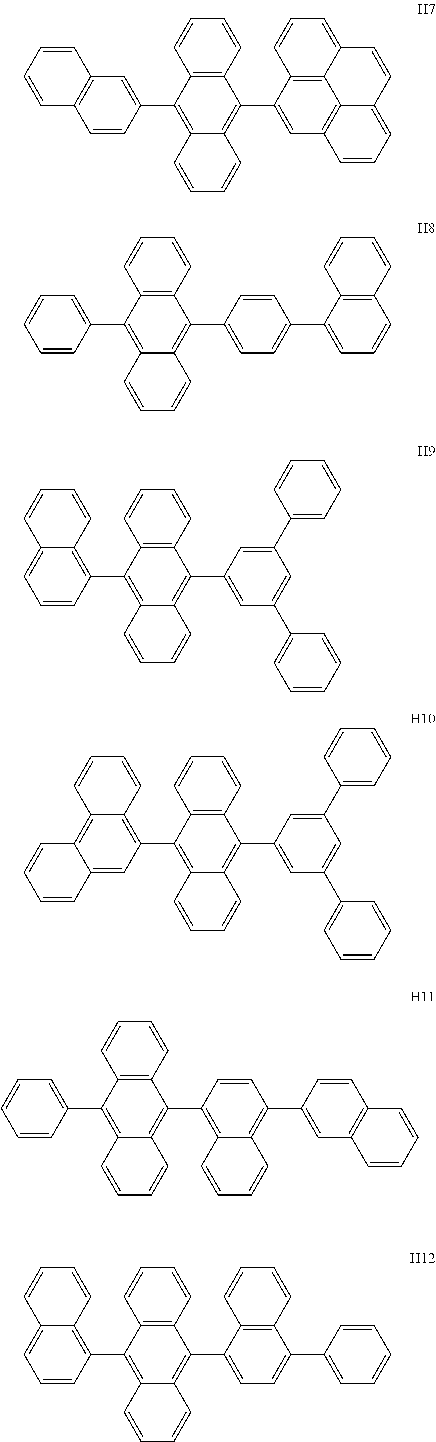

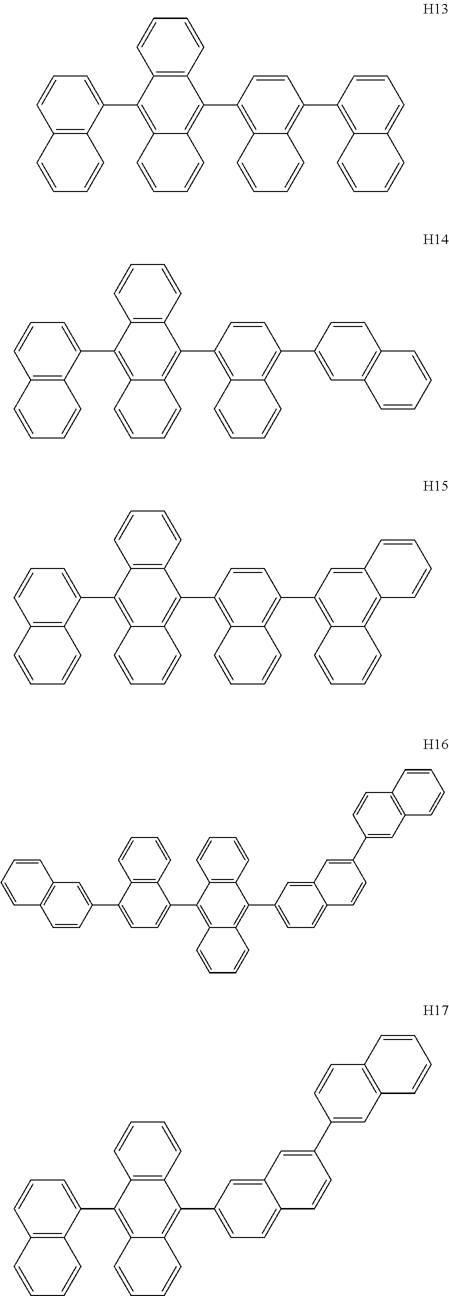

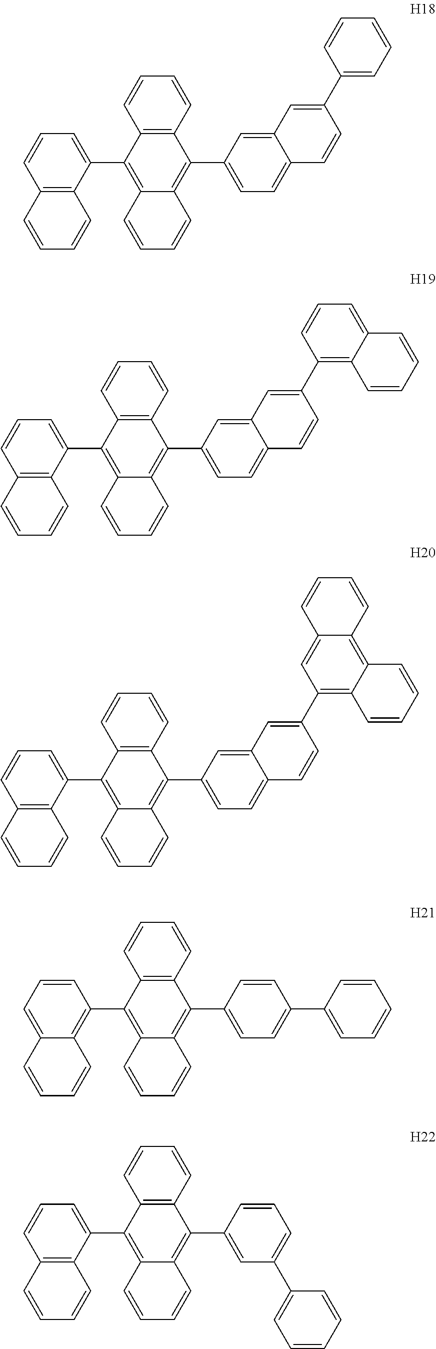

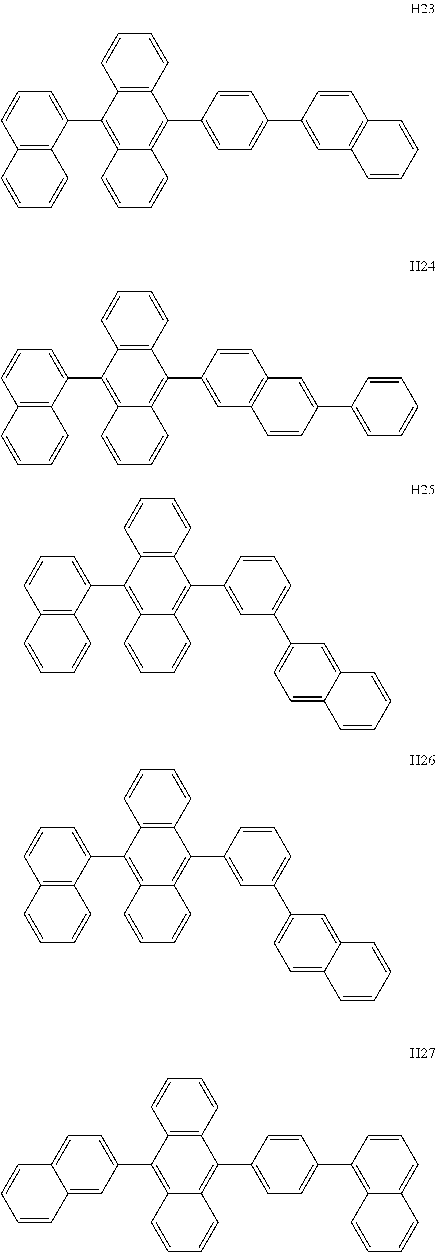

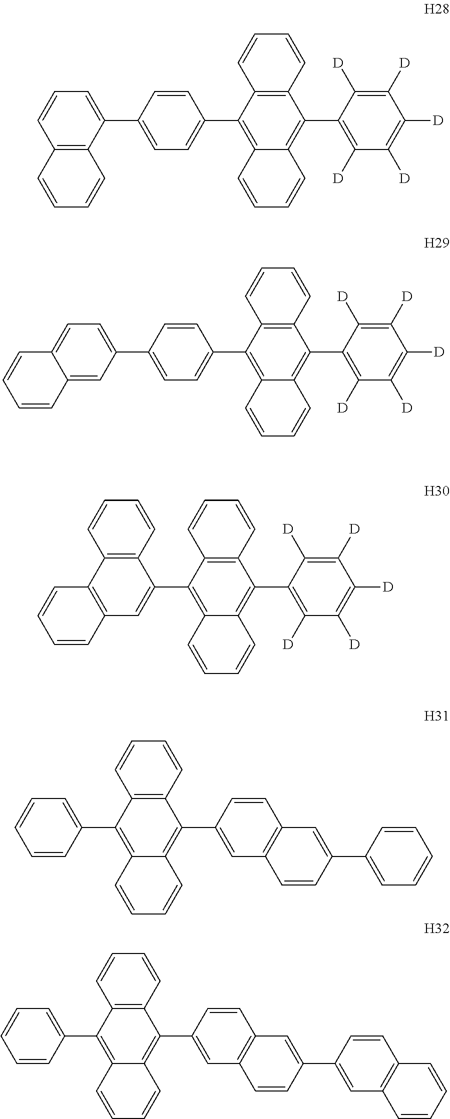

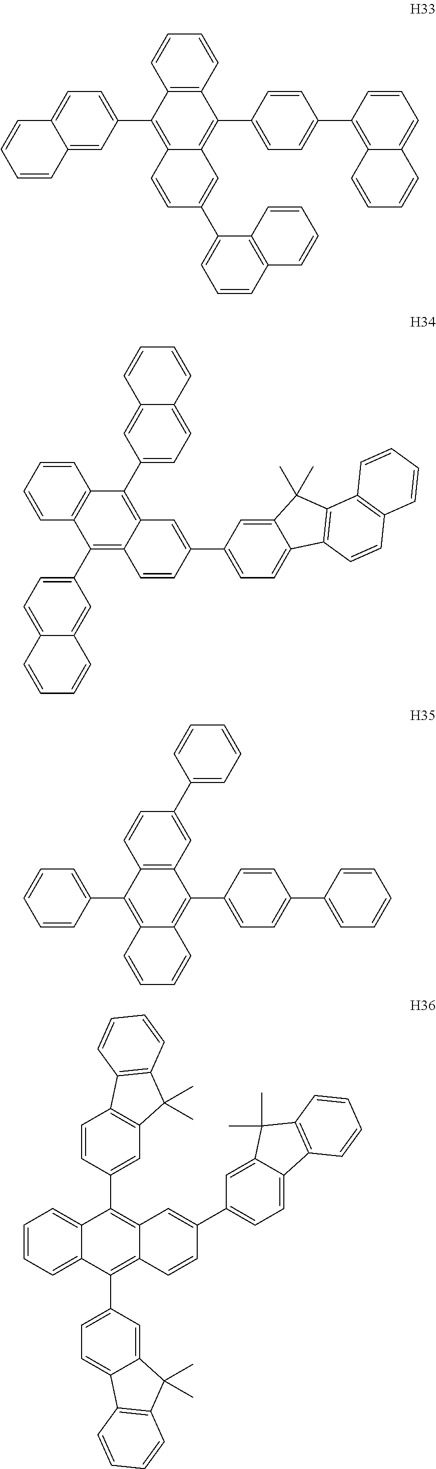

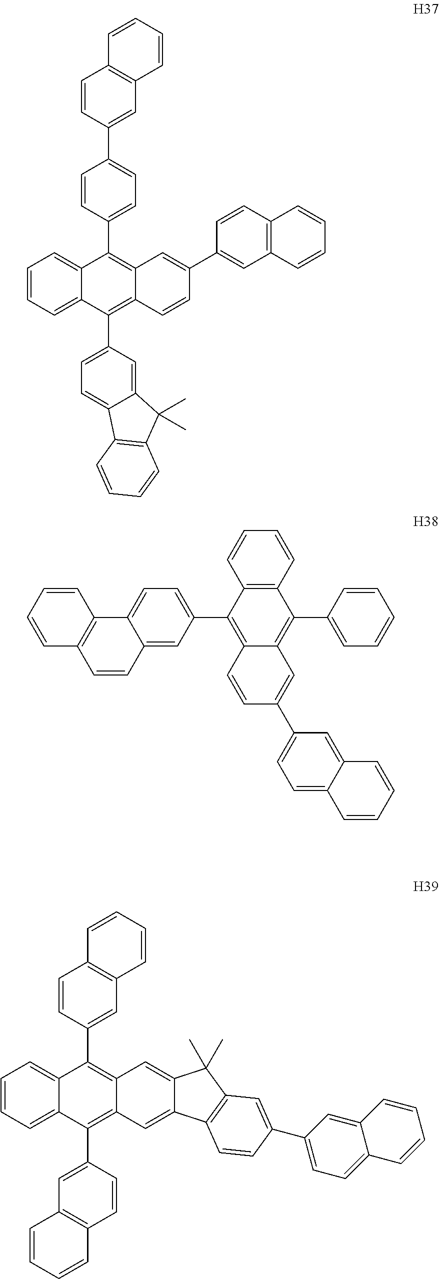

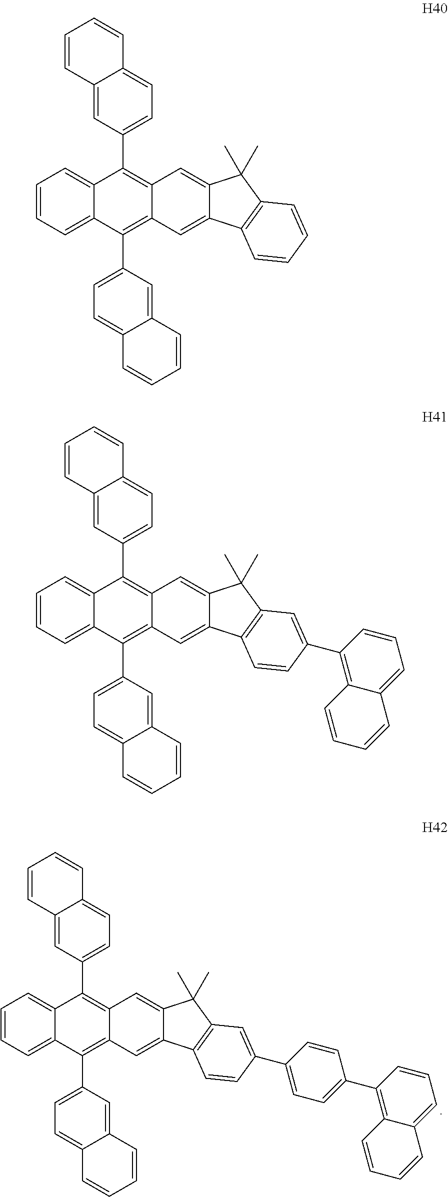

In some embodiments, the host may include at least one selected from Compounds H43 to H49 below, but is not limited thereto.

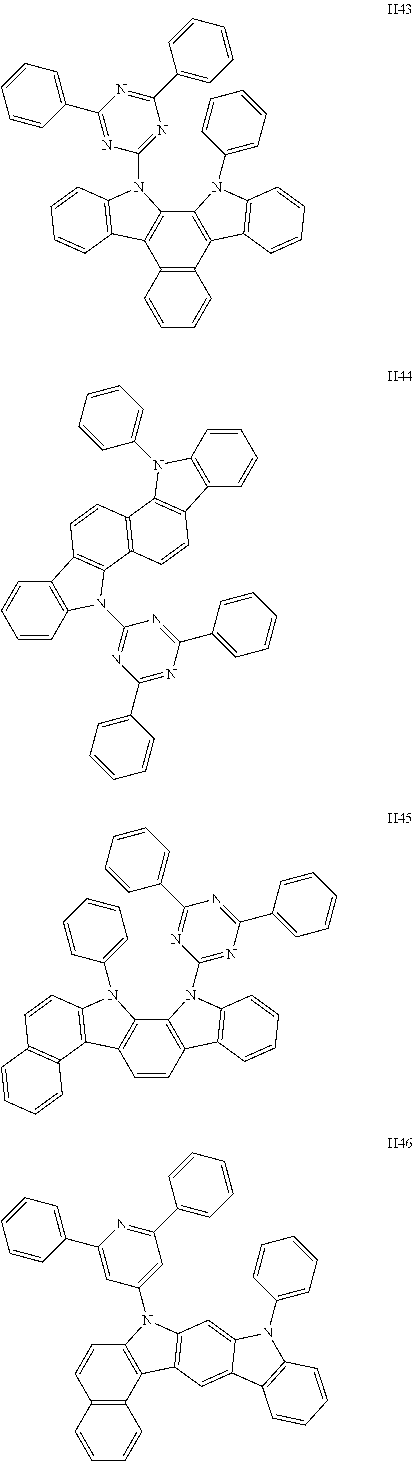 [SMILES: c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-c3ccccc3)nc(-n3c4ccccc4c4ccc5c(ccc6c7ccccc7n(-c7ccccc7)c65)c43)n2)cc1 ; c1ccc(-c2nc(-c3ccccc3)nc(-n3c4ccccc4c4ccc5c6c7ccccc7ccc6n(-c6ccccc6)c5c43)n2)cc1 ; c1ccc(-c2cc(-n3c4cc5c(cc4c4c6ccccc6ccc43)c3ccccc3n5-c3ccccc3)cc(-c3ccccc3)n2)cc1]

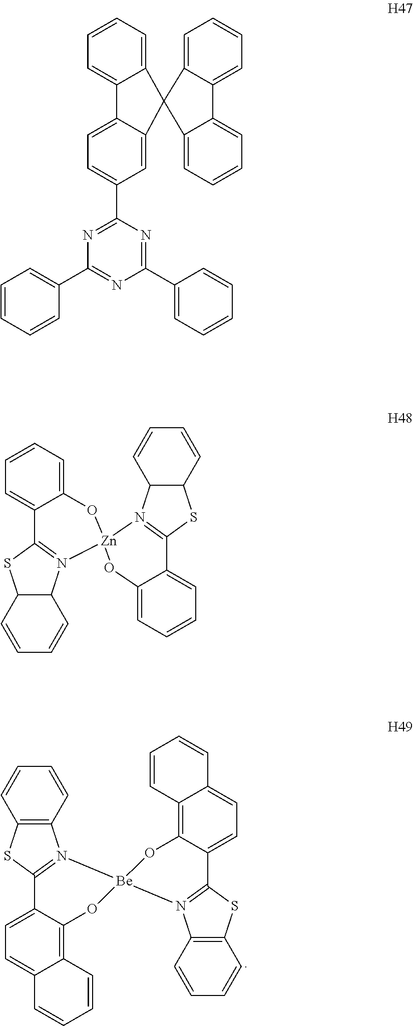

The dopant may include the compound of Formula 1 according to one or more embodiments of the present invention.

An amount of the dopant included in the emission layer may be, for example, about 0.01 parts to about 30 parts by weight, based on 100 parts by weight of the host.

A thickness of the emission layer may be about 100 Å to about 1,000 Å, for example, about 200 Å to about 600 Å. When the thickness of the emission layer is within any of these range, excellent light emission characteristics may be obtained without a substantial increase in driving voltage.

The electron transport region may be positioned on the emission layer.

The electron transport region may include, for example, at least one selected from an HBL, an ETL, and an EIL, but is not limited thereto.

When the electron transport region includes a HBL, the HBL may be formed on the emission layer by using one or more suitable methods, such as vacuum deposition, spin coating casting, an LB method, an ink-jet printing, a laser-printing, and/or an LITI method. When the HBL is formed by vacuum deposition and/or spin coating, deposition and coating conditions for forming the HBL may be similar to the deposition and coating conditions for forming the HIL.

The HBL may include, for example, at least one selected from BCP and Bphen below, but is not limited thereto.

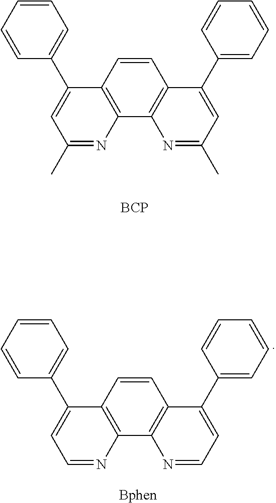

A thickness of the HBL may be about 20 Å to about 1,000 Å, for example, about 30 Å to about 300 Å. When the thickness of the HBL is within any of these ranges, excellent hole blocking characteristics may be obtained without a substantial increase in driving voltage.

The electron transport region may have a structure of ETL/EIL or a structure of HBL/ETL/EIL, where the layers of each structure are sequentially stacked on the emission layer in the stated order, but the structure of the electron transport region is not limited thereto.

According to some embodiments of the present invention, the organic layer 150 of the organic light-emitting device 10 may include the electron transport region between the emission layer and the second electrode 190. The electron transport region may include an ETL. Here, the ETL may include a plurality of layers. For example, the electron transport region may include an ETL including a first electron transport layer and a second electron transport layer.

The ETL may include at least one selected from BCP and Bphen (illustrated above) and Alq₃, Balq, TAZ, and NTAZ (illustrated below):

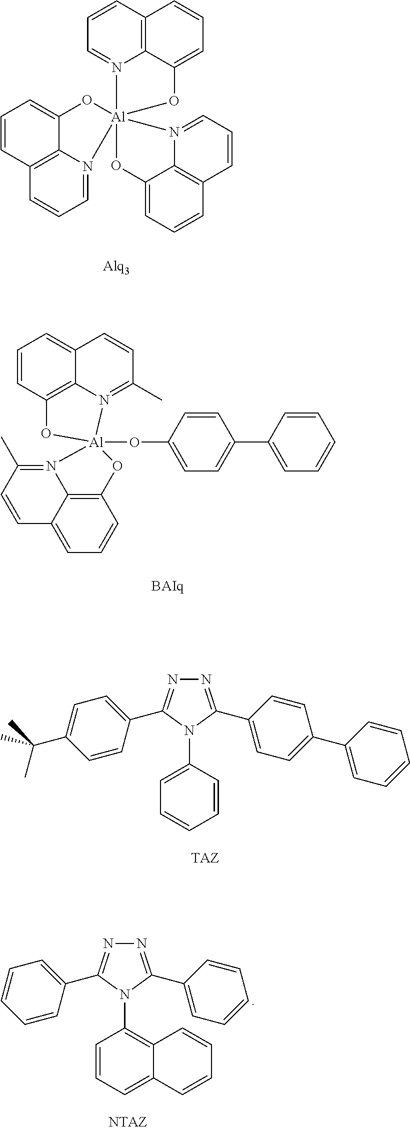

In some embodiments, the ETL may include at least one selected from a compound represented by Formula 601 and a compound represented by Formula 602 below:

Ar₆₀₁-[(L₆₀₁)ₓₑ₁-E₆₀₁]ₓₑ₂. Formula 601

In Formula 601,

Ar₆₀₁ may be selected from a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene;

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ heterocycloalkyl group, a C₃-C₁₀ cycloalkenyl group, a C₃-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, and —Si(Q₃₀₁)(Q₃₀₂)(Q₃₀₃) (where Q₃₀₁ to Q₃₀₃ may each independently be selected from hydrogen, a C₁-C₆₀ alkyl group, a C₂-C₆₀ alkenyl group, a C₆-C₆₀ aryl group, and a C₁-C₆₀ heteroaryl group);

L₆₀₁ may be understood by referring to the description provided in connection with L₂₀₃;

E₆₀₁ may be selected from:

a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group; and

a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group;

xe1 may be selected from 0, 12 and 3; and

xe2 may be selected from 1, 2, 3, and 4.

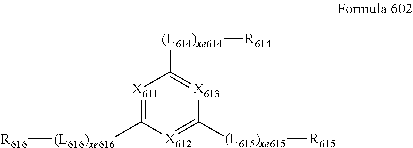

In Formula 602,

X₆₁₁ may be N or C-(L₆₁₁)ₓₑ₆₁₁-R₆₁₁, X₆₁₂ may be N or C-(L₆₁₂)ₓₑ₆₁₂-R₆₁₂, and X₆₁₃ may be N or C-(L₆₁₃)ₓₑ₆₁₃-R₆₁₃, and at least one of X₆₁₁ to X₆₁₃ may be N;

L₆₁₁ to L₆₁₆ may each independently be understood by referring to the description provided in connection with L₂₀₃;

R₆₁₁ to R₆₁₆ may each independently be selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

xe611 to xe616 may each independently be selected from 0, 1, 2, and 3.

The compound of Formula 601 and the compound of Formula 602 may be each independently selected from Compounds ET1 to ET15 below:

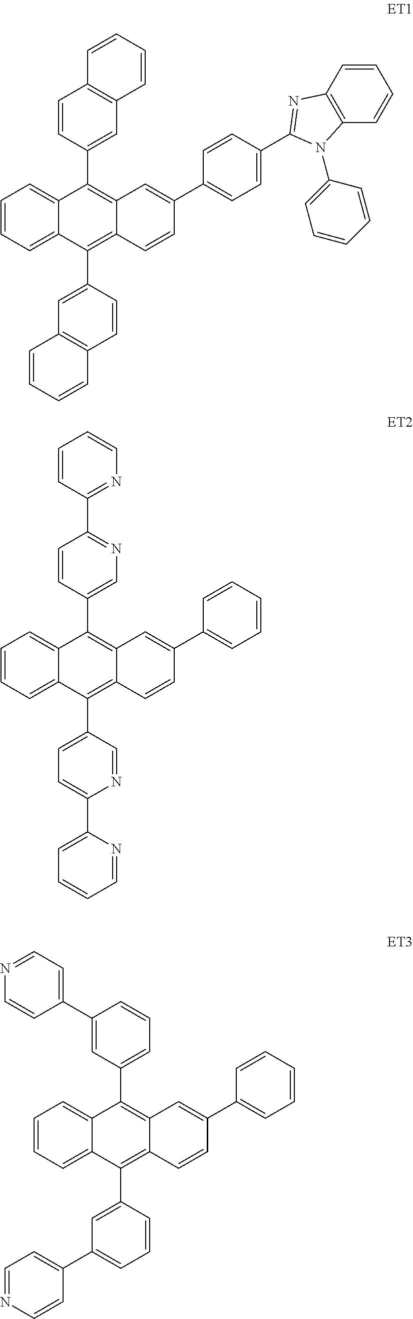

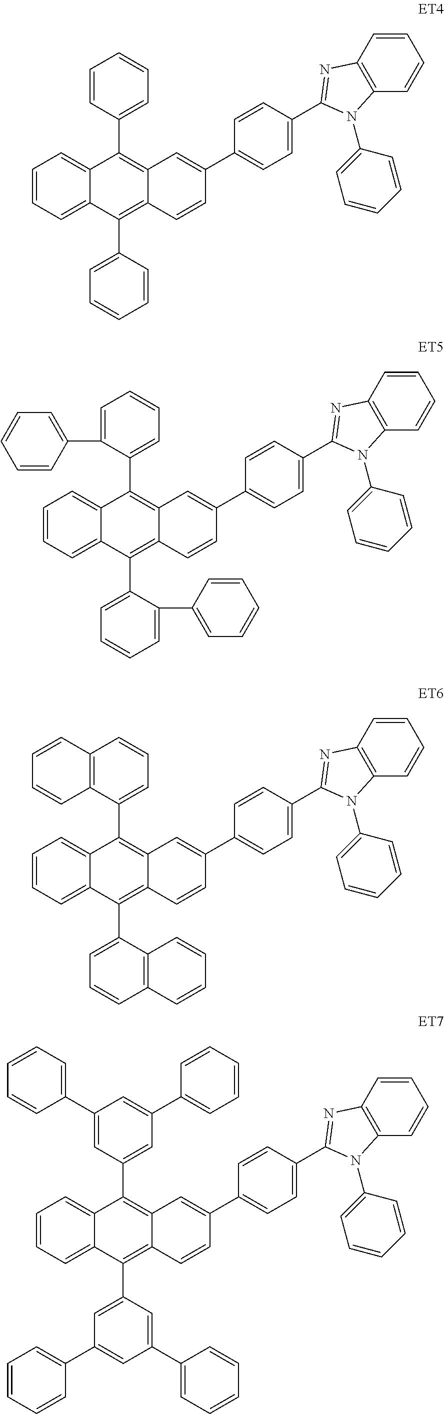 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

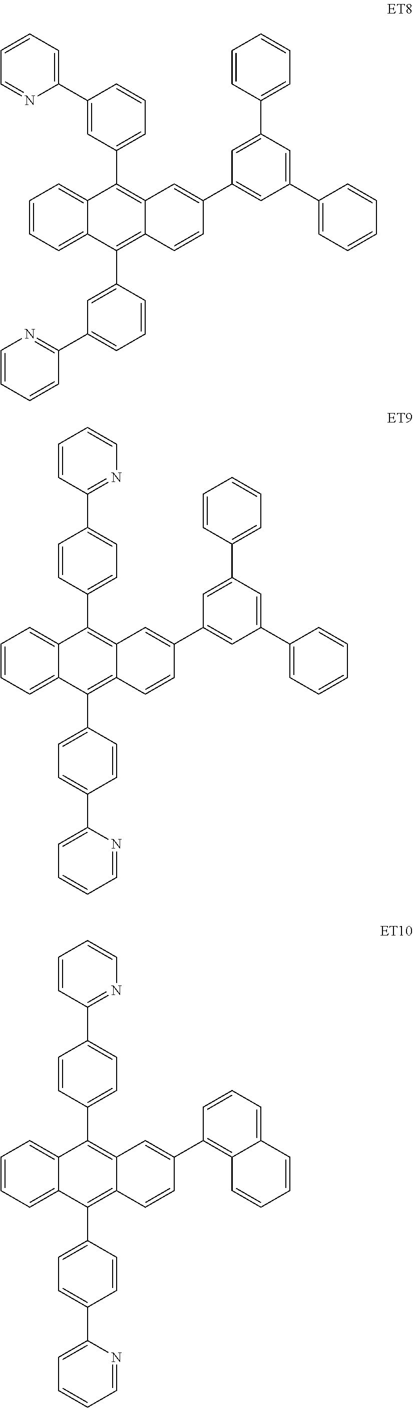

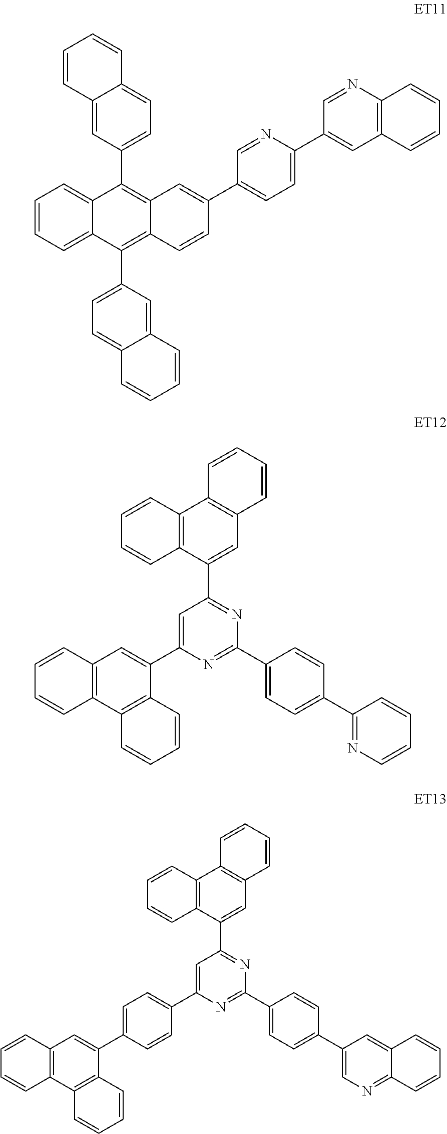 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1]

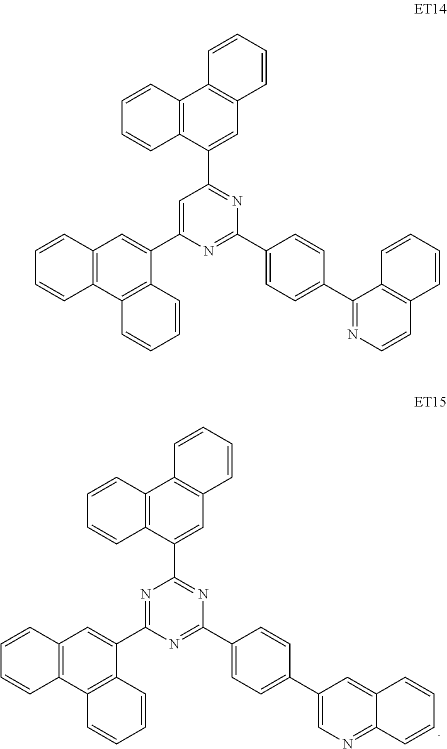 [SMILES: c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

A thickness of the ETL may be about 100 Å to about 1,000 Å, for example, about 150 Å to about 500 Å. When the thickness of ETL is within any of these ranges, satisfactory electron transporting characteristics may be obtained without a substantial increase in driving voltage.

The ETL may further include, in addition to the materials described above, a metal-containing material.

The metal-containing material may include a lithium (Li) complex. The Li complex may include, for example, Compound ET-D1 (lithium quinolate, LiQ) or ET-D2 below:

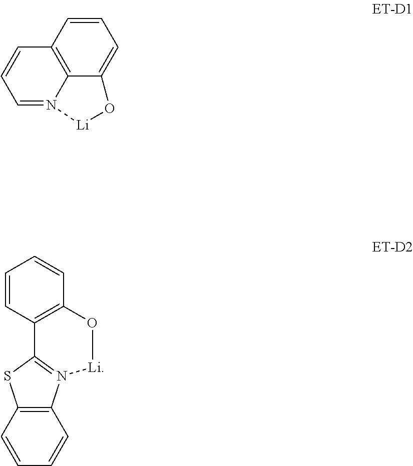

The electron transport region may include an EIL capable of facilitating electron injection from the second electrode 190.

The EIL may be formed on the ETL by using one or more suitable methods, such as vacuum deposition, spin coating casting, an LB method, an ink-jet printing, a laser-printing, and/or an LITI method. When the EIL is formed by vacuum deposition and/or spin coating, the deposition and coating conditions for the EIL may be similar to the deposition and coating conditions for the HIL.

The EIL may include at least one selected from LiF, NaCl, CsF, Li₂O, BaO, and LiQ.

A thickness of the EIL may be about 1 Å to about 100 Å, for example, about 3 Å to about 90 Å. When the thickness of the EIL is within any of these ranges, satisfactory electron transporting characteristics may be obtained without a substantial increase in driving voltage.

The second electrode 190 may be positioned on the organic layer 150. The second electrode 190 may be a cathode (e.g., an electron injection electrode). In this regard, a material for forming the second electrode 190 may be a material having a low work function, and non-limiting examples thereof may include a metal, an alloy, an electrically conductive compound, and mixtures thereof. Non-limiting examples of the material for forming the second electrode 190 may include lithium (Li), magnesium (Mg), aluminum (Al), aluminum-lithium (Al—Li), calcium (Ca), magnesium-indium (Mg—In), and magnesium-silver (Mg—Ag). In some embodiments, the material for forming the second electrode 190 may include ITO or IZO. The second electrode 190 may be a reflective electrode, a semi-transmissive electrode, or a transmissive electrode.

According to one or more embodiments of the present invention, the organic layer 150 of the organic light-emitting device 10 may be formed by vacuum deposition using (utilizing) the compound according to the present embodiments, or by coating the compound prepared as a solution.

According to one or more embodiments of the present invention, an organic light-emitting device may be loaded onto various types (kinds) of flat display devices, for example, a passive matrix organic light-emitting display device and/or an active matrix organic light-emitting display device. When the organic light-emitting device is loaded onto an active matrix organic light-emitting display device, the first electrode 110 positioned on the substrate of the organic light-emitting device 10 may be electrically connected to a source electrode or a drain electrode of a thin film transistor as a pixel electrode. In addition, the organic light-emitting device may be included in a flat display device that may display images on both sides.

Hereinabove, the organic light-emitting device was described with reference to the drawing, but embodiments of the present invention are not limited thereto.

Hereinafter, descriptions of representative substituents as used herein are provided. Carbon numbers defined for the substituents are not intended to limit properties of the substituents, and substituents that are not defined herein may be defined according to their general description known to those of ordinary skill in the art.

As used herein, the C₁-C₆₀ alkyl group refers to a linear or branched aliphatic C₁-C₆₀ hydrocarbon monovalent group, and non-limiting examples thereof include a methyl group, an ethyl group, a propyl group, an isobutyl group, a sec-butyl group, a ter-butyl group, a pentyl group, an iso-amyl, and a hexyl group. As used herein, the C₁-C₆₀ alkylene group refers to a divalent group having the same structure as the C₁-C₆₀ alkyl group.

As used herein, the C₁-C₆₀ alkoxy group refers to a monovalent group having a formula of —OA₁₀₁ (where A₁₀₁ is the C₁-C₆₀ alkyl group), and non-limiting examples thereof include a methoxy group, an ethoxy group, and an isopropyloxy group.

As used herein, the C₂-C₆₀ alkenyl group refers to a hydrocarbon group having at least one carbon-carbon double bond at one or more positions along a carbon chain of the C₂-C₆₀ alkyl group (e.g., in the middle or at either terminal end of the C₂-C₆₀ alkyl group), and non-limiting examples thereof include an ethenyl group, a propenyl group, and a butenyl group. As used herein, the C₂-C₆₀ alkenylene group refers to a divalent group having the same structure as the C₂-C₆₀ alkenyl group.

As used herein, the C₂-C₆₀ alkynyl group refers to a hydrocarbon group having at least one carbon-carbon triple bond at one or more positions along a carbon chain of the C₂-C₆₀ alkyl group (e.g., in the middle or at either terminal end of the C₂-C₆₀ alkyl group), and non-limiting examples thereof include an ethynyl group and a propynyl group. As used herein, the C₂-C₆₀ alkynylene group refers to a divalent group having the same structure as the C₂-C₆₀ alkynyl group.

As used herein, the C₃-C₁₀ cycloalkyl group refers to a saturated C₃-C₁₀ monovalent hydrocarbon monocyclic group, and non-limiting examples thereof include a cyclopropyl group, a cyclobutyl group, a cyclopentyl group, a cyclohexyl group, and a cycloheptyl group. As used herein, the C₃-C₁₀ cycloalkylene group refers to a divalent group having the same structure as the C₃-C₁₀ cycloalkyl group.

As used herein, the C₂-C₁₀ heterocycloalkyl group refers to a C₂-C₁₀ monovalent monocyclic group including at least one heteroatom selected from N, O, P, and S as a ring-forming atom (and carbon atoms as the remaining ring-forming atoms), and non-limiting examples thereof include a tetrahydrofuranyl group and a tetrahydrothiophenyl group. As used herein, the C₂-C₁₀ heterocycloalkylene group refers to a divalent group having the same structure as the C₂-C₁₀ heterocycloalkyl group.

As used herein, the C₃-C₁₀ cycloalkenyl group refers to a C₃-C₁₀ monovalent monocyclic group that has at least one double bond in a ring, but does not have aromaticity, and non-limiting examples thereof include a cyclopentenyl group, a cyclohexenyl group, and a cycloheptenyl group. As used herein, the C₃-C₁₀ cycloalkenylene group refers to a divalent group having the same structure as the C₃-C₁₀ cycloalkenyl group.

As used herein, the C₂-C₁₀ heterocycloalkenyl group refers to a C₂-C₁₀ monovalent monocyclic group including at least one heteroatom selected from N, O, P, and S as a ring-forming atom (and carbon atoms as the remaining ring-forming atoms), and includes at least one double bond in a ring. Non-limiting examples of the C₂-C₁₀ heterocycloalkenyl group include a 2,3-hydrofuranyl group and a 2,3-hydrothiophenyl group. As used herein, the C₂-C₁₀ heterocycloalkenylene group refers to a divalent group having the same structure as the C₂-C₁₀ heterocycloalkenyl group.

As used herein, the C₆-C₆₀ aryl group refers to a monovalent group having a C₆-C₆₀ carbocyclic aromatic system, and the C₆-C₆₀ arylene group refers to a divalent group having a C₆-C₆₀ carbocyclic aromatic system. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and/or the C₆-C₆₀ arylene group include 2 or more rings, the 2 or more rings may be respectively fused to each other.

As used herein, the C₁-C₆₀ heteroaryl group refers to a monovalent group having a C₁-C₆₀ carbocyclic aromatic system including at least one heteroatom selected from N, O, P, and S as a ring-forming atom (and carbon atoms as the remaining ring-forming atoms), and the C₁-C₆₀ heteroarylene group refers to a divalent group having a C₁-C₆₀ carbocyclic aromatic system including at least one heteroatom selected from N, O, P, and S as a ring-forming atom (and carbon atoms as the remaining ring-forming atoms). Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and/or the C₁-C₆₀ heteroarylene group include 2 or more rings, the 2 or more rings may be respectively fused to each other.

As used herein, the C₆-C₆₀ aryloxy group refers to a monovalent group having a formula of —OA₁₀₂ (where A₁₀₂ is the C₆-C₆₀ aryl group), and the C₆-C₆₀ arylthio group refers to a monovalent group having a formula of —SA₁₀₃ (where A₁₀₃ is the C₆-C₆₀ aryl group).

As used herein, the monovalent non-aromatic condensed polycyclic group refers to a monovalent group having 2 or more rings that are fused to each other, including only carbon atoms as ring-forming atoms (for example, carbon number may be 8 to 60), where the molecule does not have overall aromaticity. Non-limiting examples of the monovalent non-aromatic condensed polycyclic include a fluorenyl group and the like. As used herein, the divalent non-aromatic condensed polycyclic group refers to a divalent group having the same structure as the monovalent non-aromatic condensed polycyclic group.

As used herein, the monovalent non-aromatic condensed heteropolycyclic group refers to a monovalent group having 2 or more rings that are fused to each other, including at least one heteroatom selected from N, O, P, and S as a ring-forming atom, and carbon atoms as the remaining ring-forming atoms (for example, carbon number may be 2 to 60), where the molecule does not have overall aromaticity. Non-limiting examples of the monovalent non-aromatic condensed heteropolycyclic group include a carbazolyl group and the like. As used herein, the divalent non-aromatic condensed heteropolycyclic group refers to a divalent group having the same structure as the monovalent non-aromatic hetero-condensed polycyclic group.

As used herein, at least one substituent of the substituted C₃-C₁₀ cycloalkylene group, the substituted C₂-C₁₀ heterocycloalkylene group, the substituted C₃-C₁₀ cycloalkenylene group, the substituted C₂-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), and —B(Q₁₆)(Q₁₇);

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅), and —B(Q₃₆)(Q₃₇),

where Q₁₁ to Q₁₇, Q₂₁ to Q₂₇, and Q₃₁ to Q₃₇ may each independently be selected from hydrogen, deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

For example, at least one substituent of the substituted C₃-C₁₀ cycloalkylene group, the substituted C₂-C₁₀ heterocycloalkylene group, the substituted C₃-C₁₀ cycloalkenylene group, the substituted C₂-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, —N(Q₁₁)(Q₁₂), —Si(Q₁₃)(Q₁₄)(Q₁₅), and —B(Q₁₆)(Q₁₇);

a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group;

a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅), and —B(Q₃₆)(Q₃₇),

where Q₁₁ to Q₁₇, Q₂₁ to Q₂₇, and Q₃₁ to Q₃₇ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group.

The term “Ph” used herein refers to a phenyl group, the term “Me” used herein refers to a methyl group, the term “Et” used herein refers to an ethyl group, and the term “ter-Bu” or “Bu^{t}” used herein refers to a tert-butyl group.

Hereinafter, the organic light-emitting device according to one or more embodiments of the present invention will be described in greater detail with reference to the following examples.

### Synthesis Example 1

### Synthesis of Intermediate A

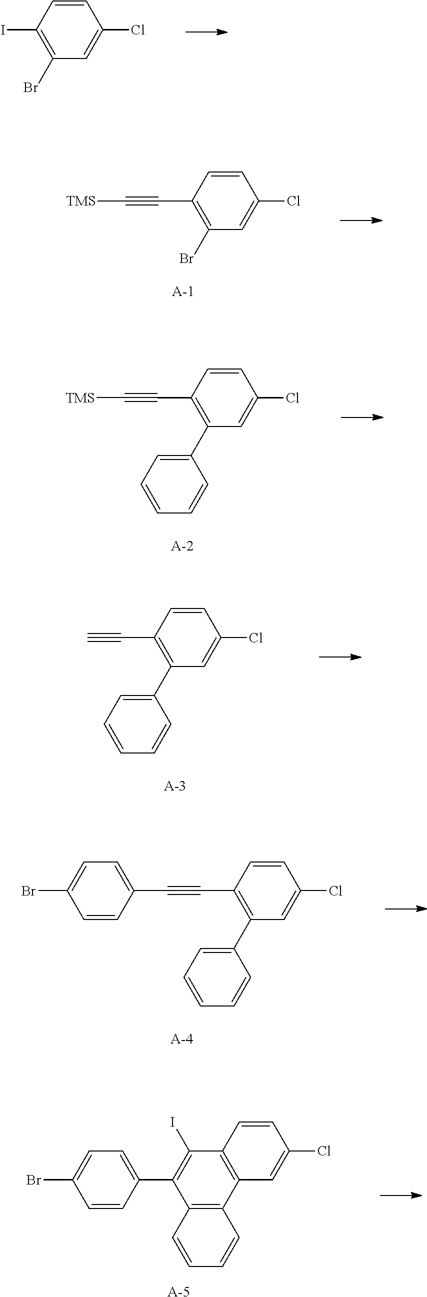

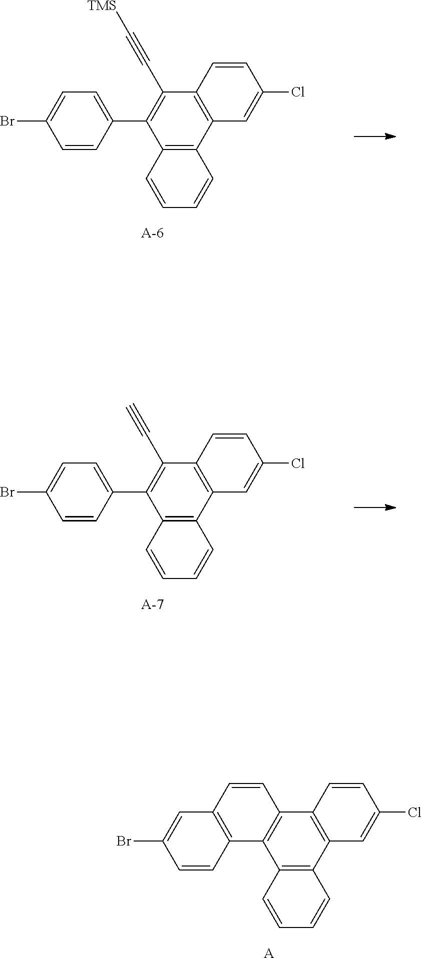

### Synthesis of Intermediate A-1

6.34 g (20 mmol) of 2-bromo-4-chloro-1-iodobenzene, 0.190 g (1 mmol) of CuI, 1.155 g (1 mmol) of (Ph3)4Pd, and 1.96 g (20 mmol) of ethynyltrimethylsilane were dissolved in 200 ml of anhydrous THF under nitrogen atmosphere, and then, 3.066 g (30 mmol) of triethylamine was added thereto. After 3 hours, the mixed solution was extracted three times, each using 100 ml of water and 10 ml of diethylether. An organic layer obtained therefrom was dried by using magnesium sulfate, and the residues obtained by evaporating the solvent were separated-purified by silica gel column chromatography, so as to obtain 5.113 g (18 mmol, yield: 90%) of Intermediate A-1.

### Synthesis of Intermediate A-2

5.113 g (18 mmol) of Intermediate A-1, 2.44 g (20 mmol) of phenylboronic acid, 1.155 g (1 mmol) of Pd(PPh₃)₄, and 2.762 g (20 mmol) of K₂CO₃ were dissolved in 200 ml of a mixed solution of THF/H2O (at a volume ratio of 2/1) under nitrogen atmosphere, and then, the mixed solution was stirred at a temperature of 80° C. for 12 hours. The resulting reaction solution was cooled to room temperature, and then, extracted three times, each using 50 ml of water and 150 ml of ethylether. An organic layer obtained therefrom was dried by using magnesium sulfate, and the residues obtained by evaporating the solvent were separated-purified by silica gel column chromatography, so as to obtain 4.26 g (15 mmol, yield: 83%) of Intermediate A-2.

### Synthesis of Intermediate A-3

4.26 g (15 mmol) of Intermediate A-2 and 0.800 g (20 mmol) of sodium hydroxide were dissolved and added in 100 ml of methanol, and then, the mixed solution was stirred at a temperature of 60° C. for 1 hour. The resulting reaction solution was extracted three times, each using 50 ml of water and 50 ml of ethylether. An organic layer obtained therefrom was dried by using magnesium sulfate solvent, and the residues obtained by evaporating the solvent were separated-purified by silica gel column chromatography, so as to obtain 2.968 g (14 mmol, yield: 93%) of Intermediate A-3.

### Synthesis of Intermediate A-4

3.678 g (10 mmol, yield: 71%) of Intermediate A-4 was obtained in the same (or substantially the same) manner as in synthesizing Intermediate A-1 of Synthesis Example 1, except that 1-bromo-4-iodine-benzene and Intermediate A-3 were used instead of 2-bromo-4-chloro-1-iodobenzene and ethynyltrimethylsilane, respectively.

### Synthesis of Intermediate A-5

3.678 g (10 mmol) of Intermediate A-4 was dissolved in 100 ml of dichloromethane, and then, 1.622 g of ICI was added thereto. The mixed solution was extracted three times, each using 50 ml of water and 10 ml of dichloromethane. An organic layer obtained therefrom was dried by using magnesium sulfate, and the residues obtained by evaporating the solvent were separated-purified by silica gel column chromatography, so as to obtain 4.437 g (9 mmol, yield: 90%) of Intermediate A-5.

### Synthesis of Intermediate A-6

3.756 g (8.1 mmol, yield: 90%) of Intermediate A-6 was synthesized in the same (or substantially the same) manner as in synthesizing Intermediate A-1 of Synthesis Example 1, except that Intermediate A-5 was used instead of 2-bromo-4-chloro-1-iodobenzene.

### Synthesis of Intermediate A-7

2.741 g (7 mmol, yield: 87%) of Intermediate A-7 was synthesized in the same (or substantially the same) manner as in synthesizing Intermediate A-3 of Synthesis Example 1, except that Intermediate A-6 was used instead of Intermediate A-2.

### Synthesis of Compound A

2.741 g (7 mmol) of Intermediate A-7 and 0.092 g (0.35 mmol) of PtCl₂ were dissolved in 50 ml of toluene, and then, the mixed solution was stirred at a temperature of 100° C. for 12 hours. The resulting reaction solution was extracted three times, each using 50 ml of water and 50 ml of ethylether. An organic layer obtained therefrom was dried by using magnesium sulfate, and the residues obtained by evaporating the solvent were separated-purified by silica gel column chromatography, so as to obtain 1.564 g (4 mmol, yield: 57%) of Compound A.

### Synthesis Example 2

### Synthesis of Compound 2

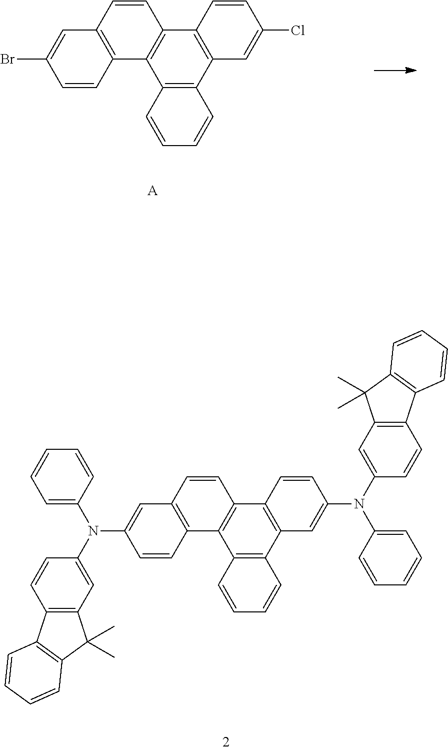

### Synthesis of Compound 2

0.391 g (1 mmol) of Intermediate A, 0.855 g (3 mmol) of 9,9-dimethyl-N-phenyl-9H-fluoren-2-amine, 0.091 g (0.1 mmol) of tris(dibenzylideneacetone)dipalladium(0) (Pd₂(dba)₃), 0.020 g (0.1 mmol) of tri-tert-butylphosphine (P(t-Bu)₃), and 0.28 g (3 mmol) of KOtBu were dissolved in 60 ml of toluene under nitrogen atmosphere, and then, the mixed solution was stirred at a temperature of 90° C. for 4 hours. The resulting reaction solution was cooled to room temperature, and then, was extracted three times, each using 50 ml of water and 50 ml of diethylether. An organic layer obtained therefrom was dried by using magnesium sulfate, and the residues obtained by evaporating the solvent were separated-purified by silica gel column chromatography, so as to obtain 0.720 g (0.85 mmol, yield: 86%) of Compound 2.

### Synthesis Example 3

### Synthesis of Compound 72

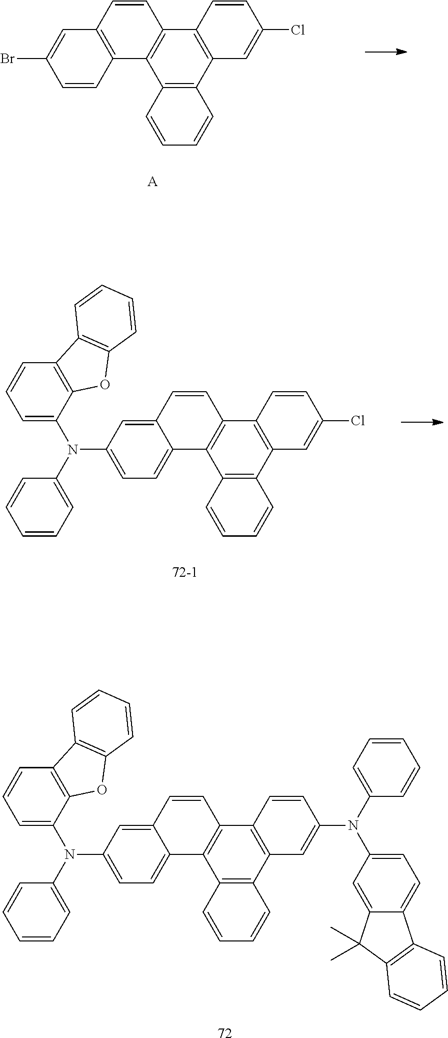

### Synthesis of Compound 72-1

0.500 g (0.87 mmol, yield: 87%) of Intermediate 72-1 was synthesized in the same (or substantially the same) manner as in synthesizing Compound 2 of Synthesis Example 2, except that N-phenyldibenzo[b,d]furan-4-amine was used instead of 9,9-dimethyl-N-phenyl-9H-fluoren-2-amine.

### Synthesis of Compound 72

0.492 g (0.6 mmol, yield: 69%) of Compound 72 was synthesized in the same (or substantially the same) manner as in synthesizing Compound 2 of Synthesis Example 2, except that Intermediate 72-1 was used instead of Intermediate A.

Additional compounds were synthesized by using appropriate intermediate compounds according to the same (or substantially the same) synthesis method as described above. Other compounds in addition to the synthesized compounds described above should be apparent to one of ordinary skill in the art by referring to methods and raw materials described above.

### Example 1

An anode was prepared by cutting a Corning 15 Ω/cm² (1,200 Å) ITO glass substrate to a size of 50 mm×50 mm×0.7 mm, ultrasonically cleaning the glass substrate by using isopropyl alcohol and pure water, for 5 minutes each, and then, irradiating UV light for 30 minutes thereto, and exposing to ozone to clean. Then, the obtained anode was loaded onto a vacuum deposition apparatus.

Then, 2-TNATA was vacuum deposited on the ITO layer to form an HIL having a thickness of 600 Å. Then, 4,4′-bis[N-(1-naphthyl)-N-phenylamino]biphenyl (NPB) was vacuum deposited on the HIL to form an HTL having a thickness of 300 Å.

Next, 9,10-di-naphthalene-2-yl-anthracene (DNA or ADN) and Compound 2 were co-deposited on the HTL at a weight ratio of 98:2 to form an emission layer having a thickness of 300 Å.

Alq₃ was deposited on the emission layer to form an ETL having a thickness of 300 Å, and then, LiF was deposited on the ETL to form an EIL having a thickness of 10 Å. Then, AI was deposited on the EIL to form a second electrode (i.e., a cathode) having a thickness of 3,000 Å, thereby completing manufacturing of an organic light-emitting device.

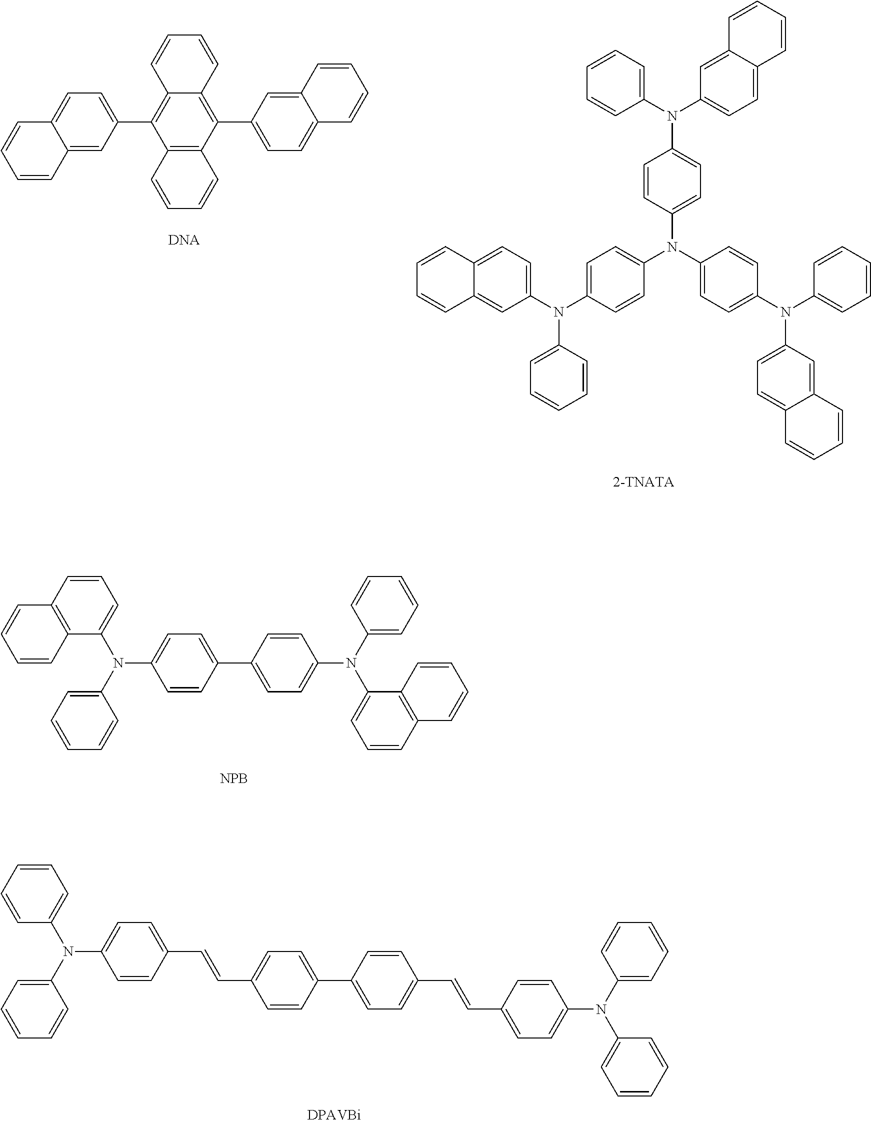

### Example 2

An organic light-emitting device was manufactured in the same (or substantially the same) manner as in Example 1, except that Compound 9 was used instead of Compound 2 to form the emission layer.

### Example 3

An organic light-emitting device was manufactured in the same (or substantially the same) manner as in Example 1, except that Compound 13 was used instead of Compound 2 to form the emission layer.

### Example 4

An organic light-emitting device was manufactured in the same (or substantially the same) manner as in Example 1, except that Compound 15 was used instead of Compound 2 to form the emission layer.

### Example 5

An organic light-emitting device was manufactured in the same (or substantially the same) manner as in Example 1, except that Compound 38 was used instead of Compound 2 to form the emission layer.

### Example 6

An organic light-emitting device was manufactured in the same (or substantially the same) manner as in Example 1, except that Compound 57 was used instead of Compound 2 to form the emission layer.

### Example 7

An organic light-emitting device was manufactured in the same (or substantially the same) manner as in Example 1, except that Compound 72 was used instead of Compound 2 to form the emission layer.

### Example 8

An organic light-emitting device was manufactured in the same (or substantially the same) manner as in Example 1, except that Compound 88 was used instead of Compound 2 to form the emission layer.

### Example 9

An organic light-emitting device was manufactured in the same (or substantially the same) manner as in Example 1, except that Compound 90 was used instead of Compound 2 to form the emission layer.

### Comparative Example 1

An organic light-emitting device was manufactured in the same (or substantially the same) manner as in Example 1, except that DPAVBi (a known blue fluorescent dopant) was used instead of Compound 2 to form the emission layer.

### Comparative Example 2

An organic light-emitting device was manufactured in the same (or substantially the same) manner as in Example 1, except that Compound 200 below was used instead of Compound 2 to form the emission layer.

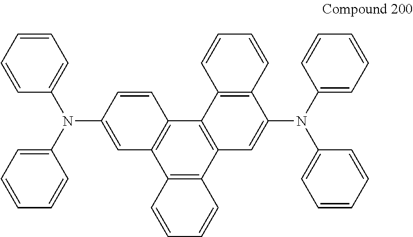 [SMILES: c1ccc(N(c2ccccc2)c2ccc3c(c2)c2ccccc2c2cc(N(c4ccccc4)c4ccccc4)c4ccccc4c32)cc1]

The characteristics of the organic light-emitting devices of Comparative Examples and Examples are shown in Table 1 below.

TABLE 1

| | | Driving | Current | | | | |
|---|---|---|---|---|---|---|---|
| | | voltage | density | Brightness | Efficiency | Emission | Half-lifespan |
| | Material | (V) | (mA/cm²) | (cd/m²) | (cd/A) | color | (hr @100 mA/cm²) |
| Example 1 | Compound 2 | 5.96 | 50 | 3,005 | 6.01 | Blue | 315 |
| Example 2 | Compound 9 | 6.02 | 50 | 2,995 | 5.99 | Blue | 320 |
| Example 3 | Compound 13 | 5.99 | 50 | 3,108 | 6.21 | Blue | 305 |
| Example 4 | Compound 15 | 6.24 | 50 | 3,069 | 6.14 | Blue | 335 |
| Example 5 | Compound 38 | 5.89 | 50 | 3,052 | 6.10 | Blue | 300 |
| Example 6 | Compound 57 | 5.90 | 50 | 3,041 | 6.08 | Blue | 290 |
| Example 7 | Compound 72 | 6.01 | 50 | 2,900 | 5.80 | Blue | 308 |
| Example 8 | Compound 88 | 6.10 | 50 | 2,950 | 5.90 | Blue | 315 |
| Example 9 | Compound 90 | 6.15 | 50 | 3,080 | 6.16 | Blue | 302 |
| Comparative | DPAVBi | 7.01 | 50 | 2,645 | 5.29 | Blue | 258 |
| Example 1 | | | | | | | |
| Comparative | 200 | 6.98 | 50 | 2,789 | 5.34 | Blue | 261 |
| Example 2 | | | | | | | |

Referring to Table 1, when the compound of Formula 1 according to embodiments of the present invention was used as a dopant in the emission layer, the resulting organic light-emitting devices of the Examples had low driving voltages and showed excellent I-V-L characteristics with significantly improved efficiencies, as compared to those of the organic light-emitting devices of Comparative Examples. In particular, organic light-emitting devices of the Examples showed excellent results in the lifespan improvement.

As described above, according to one or more embodiments of the present invention, the compounds represented by Formula 1 may have excellent emission characteristics and charge-transporting capability, and thus may be used as an emission material that is suitable for fluorescent and phosphorescent devices of all colors including but not limited to red, green, blue, and white. In this regard, an organic light-emitting device including the compounds may have high efficiency, low voltage, high brightness, and long lifespan.

It should be understood that the embodiments described therein should be considered in a descriptive sense only and not for purposes of limitation. Descriptions of features or aspects within each embodiment should typically be considered as available for other similar features or aspects in other embodiments.

While one or more embodiments have been described with reference to the drawing, it will be understood by those of ordinary skill in the art that various changes in form and details may be made therein without departing from the spirit and scope as defined by the following claims and equivalents thereof.

What is claimed is:

1. A compound represented by Formula 1: 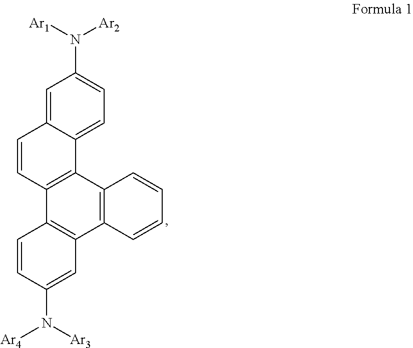
wherein, in Formula 1,
Ar₁ to Ar₄ are each independently selected from a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;
at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is selected from:
deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), and —B(Q₁₆)(Q₁₇);
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and
—N(Q₃₁)(Q₃₂), —Si(Q₃₃)(Q₃₄)(Q₃₅), and —B(Q₃₆)(Q₃₇),
wherein Q₁₁ to Q₁₇, Q₂₁ to Q₂₇, and Q₃₁ to Q₃₇ are each independently selected from hydrogen, deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

2. The compound of claim 1, wherein Ar₁ to Ar₄ in Formula 1 are each independently selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

3. The compound of claim 1, wherein the compound of Formula 1 is represented by Formula 2: 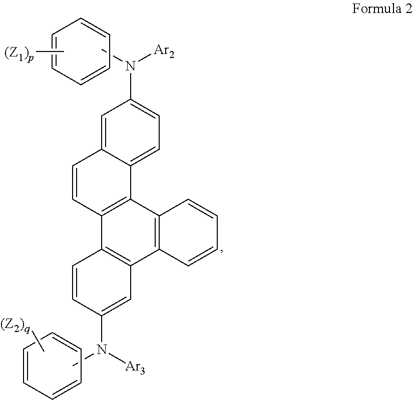
wherein, in Formula 2, Z₁ and Z₂ are each independently selected from hydrogen, deuterium, a halogen atom, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₃)(Q₃₄)(Q₃₅);
p and q are each independently an integer selected from 1 to 5, wherein when p is 2 or more, Z₁s are identical to or different from each other, and when q is 2 or more, Z₂s are identical to or different from each other; and
* indicates a binding site.

4. The compound of claim 1, wherein the compound of Formula 1 is represented by Formula 3: 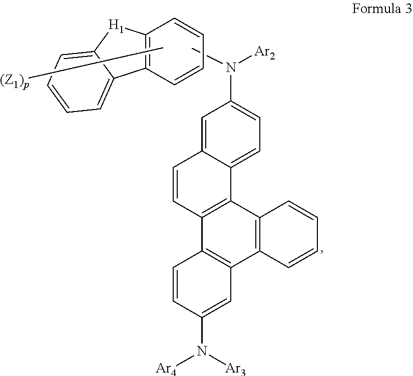
wherein, in Formula 3, H₁ is CR₁R₂, O, or S;
Z₁, R₁, and R₂ are each independently selected from hydrogen, deuterium, a halogen atom, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₃)(Q₃₄)(Q₃₅);
p is an integer selected from 1 to 7, wherein when p is 2 or more, Z₁s are identical to or different from each other; and
* indicates a binding site.

5. The compound of claim 1, wherein the compound of Formula 1 is represented by Formula 4: 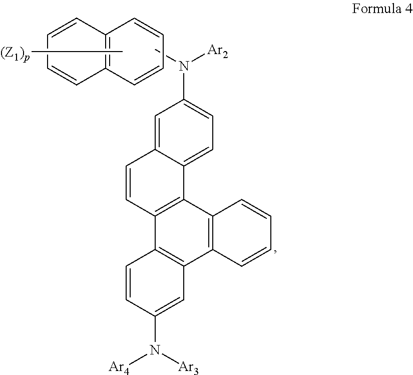
wherein, in Formula 4, Z₁ is selected from hydrogen, deuterium, a halogen atom, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₃)(Q₃₄)(Q₃₅);
p is an integer selected from 1 to 7, wherein when p is 2 or more, Z₁s are identical to or different from each other; and
* indicates a binding site.

6. The compound of claim 1, wherein the compound of Formula 1 is represented by Formula 5: 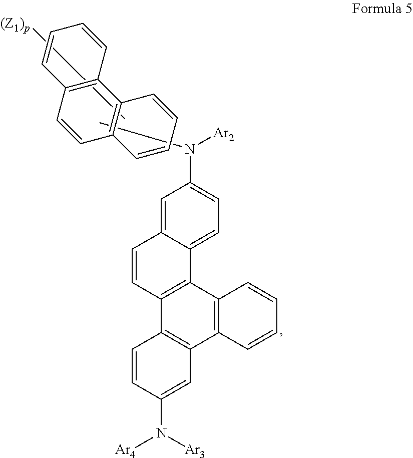
wherein, in Formula 5, Z₁ is selected from hydrogen, deuterium, a halogen atom, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₃)(Q₃₄)(Q₃₅);
p is an integer selected from 1 to 9, wherein when p is 2 or more, Z₁s are identical to or different from each other; and
* indicates a binding site.

7. The compound of claim 1, wherein Ar₁ to Ar₄ in Formula 1 are each independently selected from hydrogen, deuterium, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group; and
a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group, each substituted with at least one selected from deuterium, —F, —CI, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, —Si(Q₃₃)(Q₃₄)(Q₃₅), a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzooxazolyl group, an isobenzooxazolyl group, a triazolyl group, a tetrazolyl group, an oxadiazolyl group, a triazinyl group, a dibenzofuranyl group, a dibenzothiophenyl group, a benzocarbazolyl group and a dibenzocarbazolyl group, a thiadiazolyl group, an imidazopyridinyl group, and an imidazopyrimidinyl group.

8. The compound of claim 1, wherein Ar₁ to Ar₄ in Formula 1 are each independently selected from hydrogen, deuterium, and a compound represented by any one of Formulae 2a to 2e: 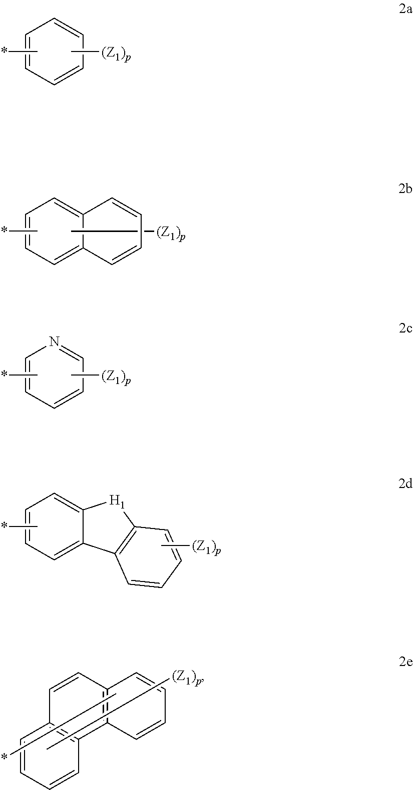
wherein, in Formulae 2a to 2e, H₁ is CR₁R₂, O, or S;
Z₁, R₁, and R₂ are each independently selected from hydrogen, deuterium, a halogen atom, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₃)(Q₃₄)(Q₃₅);
p is an integer selected from 1 to 9, wherein when p is 2 or more, Z₁s are identical to or different from each other; and
* indicates a binding site.

9. The compound of claim 1, wherein the compound of Formula 1 is selected from Compounds 1-112 below:

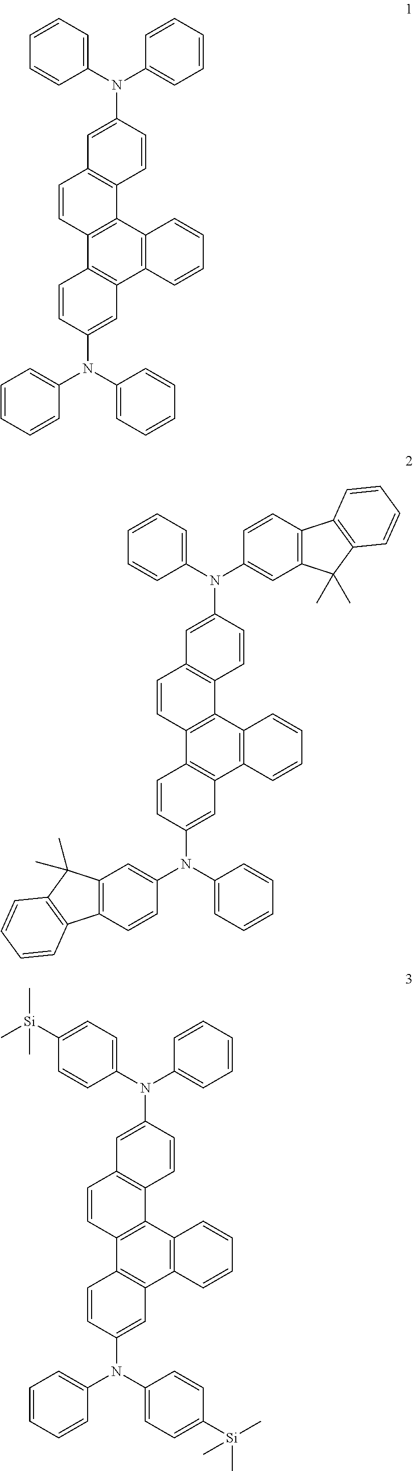

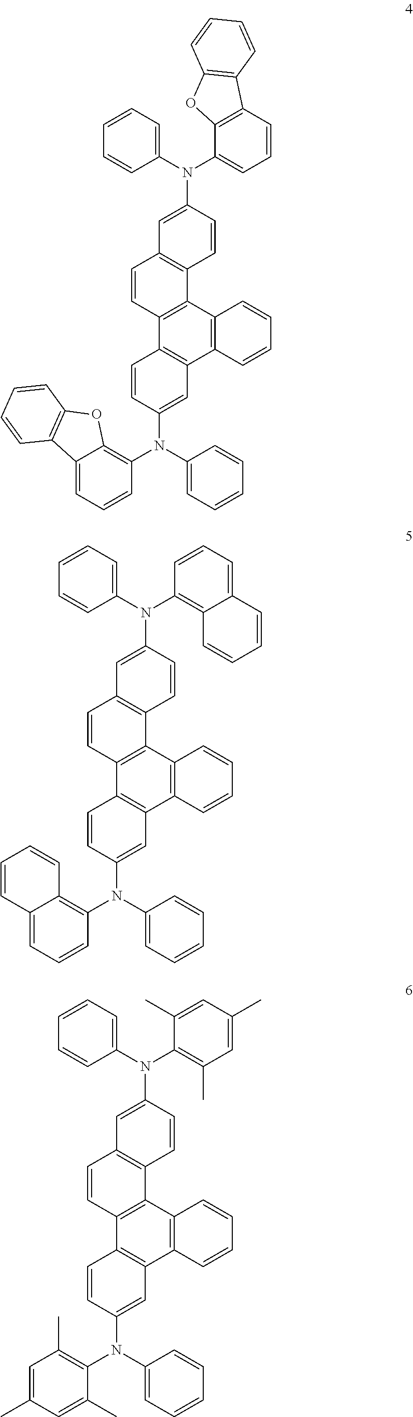

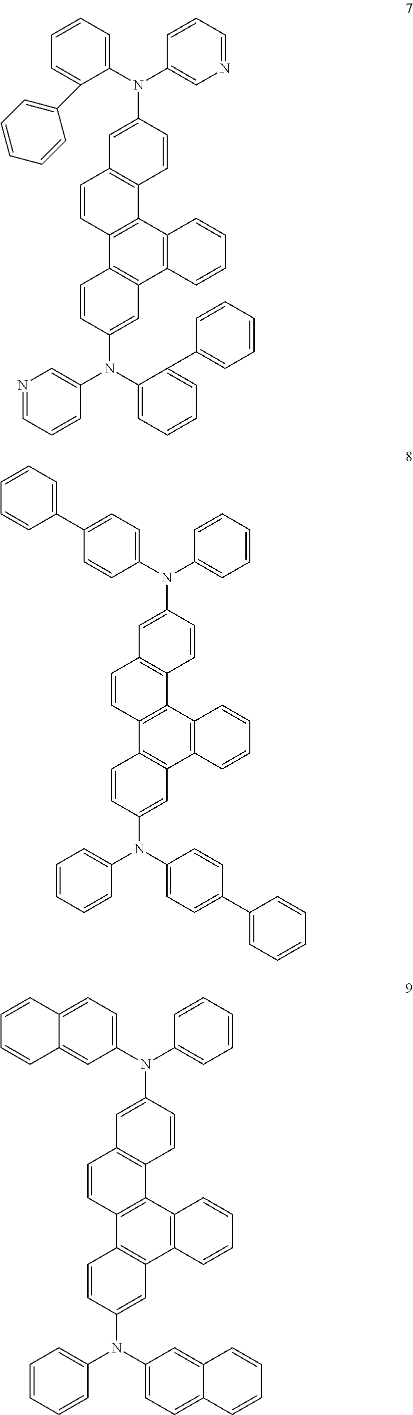

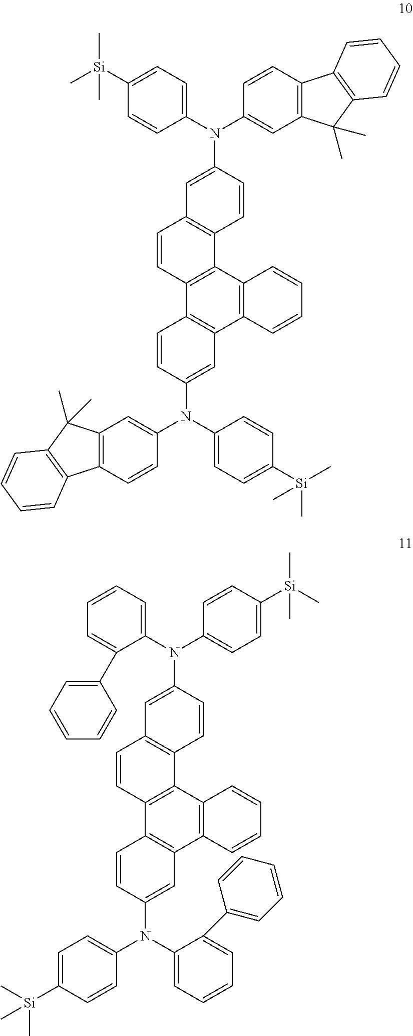

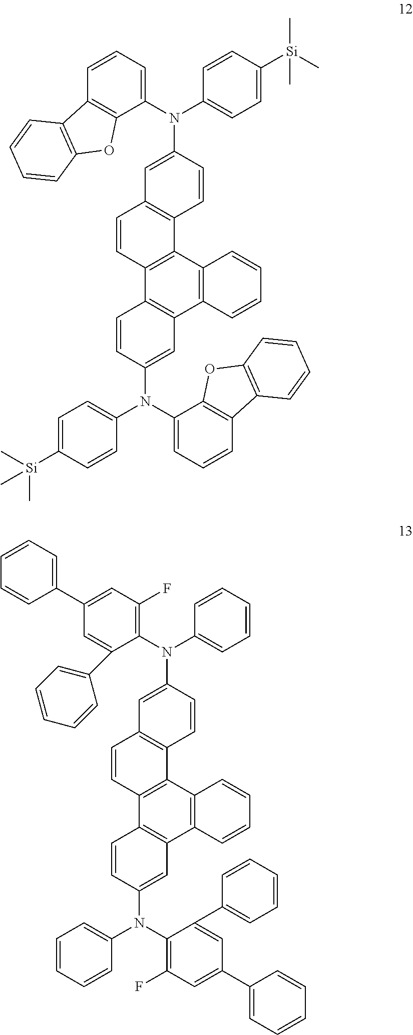

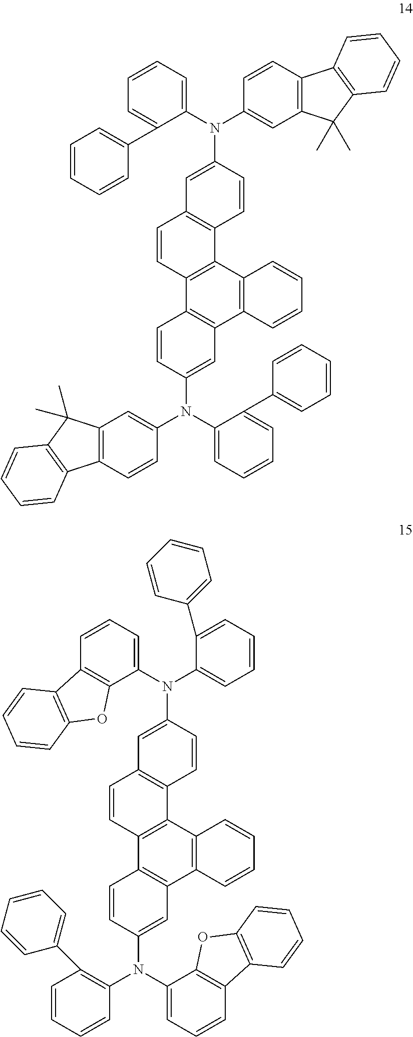 [SMILES: CC1(C)c2ccccc2-c2ccc(N(c3ccc4c(ccc5c6ccc(N(c7ccc8c(c7)C(C)(C)c7ccccc7-8)c7ccccc7-c7ccccc7)cc6c6ccccc6c45)c3)c3ccccc3-c3ccccc3)cc21 ; c1ccc(-c2ccccc2N(c2ccc3c(ccc4c5ccc(N(c6ccccc6-c6ccccc6)c6cccc7c6oc6ccccc67)cc5c5ccccc5c34)c2)c2cccc3c2oc2ccccc23)cc1]

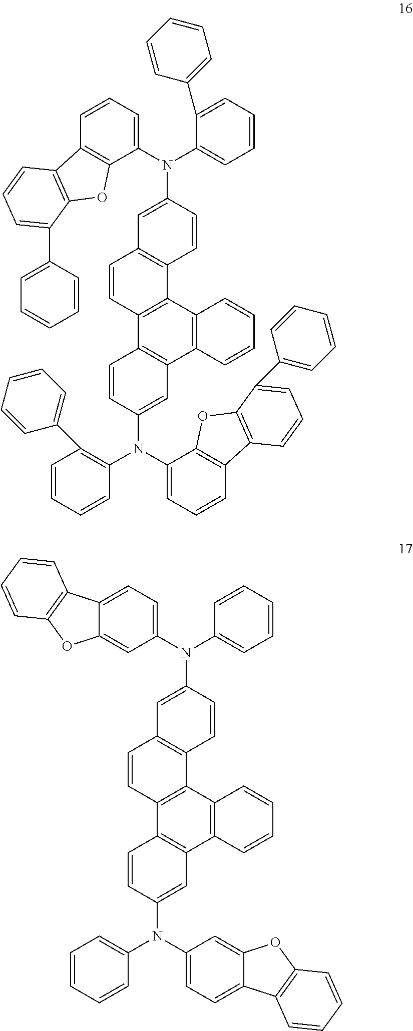 [SMILES: c1ccc(-c2ccccc2N(c2ccc3c(ccc4c5ccc(N(c6ccccc6-c6ccccc6)c6cccc7c6oc6c(-c8ccccc8)cccc67)cc5c5ccccc5c34)c2)c2cccc3c2oc2c(-c4ccccc4)cccc23)cc1 ; c1ccc(N(c2ccc3c(ccc4c5ccc(N(c6ccccc6)c6ccc7c(c6)oc6ccccc67)cc5c5ccccc5c34)c2)c2ccc3c(c2)oc2ccccc23)cc1]

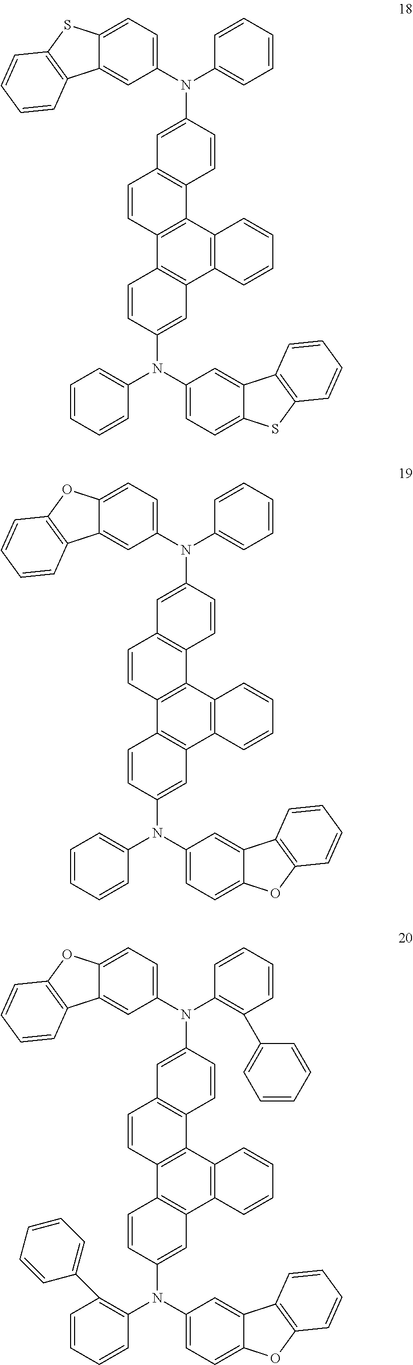 [SMILES: c1ccc(N(c2ccc3c(ccc4c5ccc(N(c6ccccc6)c6ccc7sc8ccccc8c7c6)cc5c5ccccc5c34)c2)c2ccc3sc4ccccc4c3c2)cc1 ; c1ccc(N(c2ccc3c(ccc4c5ccc(N(c6ccccc6)c6ccc7oc8ccccc8c7c6)cc5c5ccccc5c34)c2)c2ccc3oc4ccccc4c3c2)cc1 ; c1ccc(-c2ccccc2N(c2ccc3c(ccc4c5ccc(N(c6ccc7oc8ccccc8c7c6)c6ccccc6-c6ccccc6)cc5c5ccccc5c34)c2)c2ccc3oc4ccccc4c3c2)cc1]

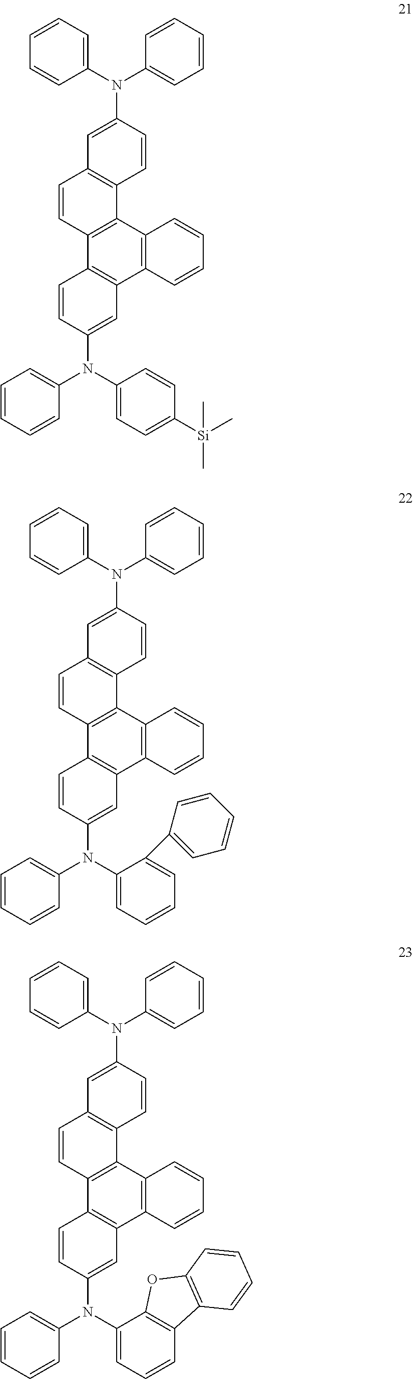 [SMILES: C[Si](C)(C)c1ccc(N(c2ccccc2)c2ccc3c(c2)c2ccccc2c2c4ccc(N(c5ccccc5)c5ccccc5)cc4ccc32)cc1 ; c1ccc(-c2ccccc2N(c2ccccc2)c2ccc3c(c2)c2ccccc2c2c4ccc(N(c5ccccc5)c5ccccc5)cc4ccc32)cc1 ; c1ccc(N(c2ccccc2)c2ccc3c(ccc4c5ccc(N(c6ccccc6)c6cccc7c6oc6ccccc67)cc5c5ccccc5c34)c2)cc1]

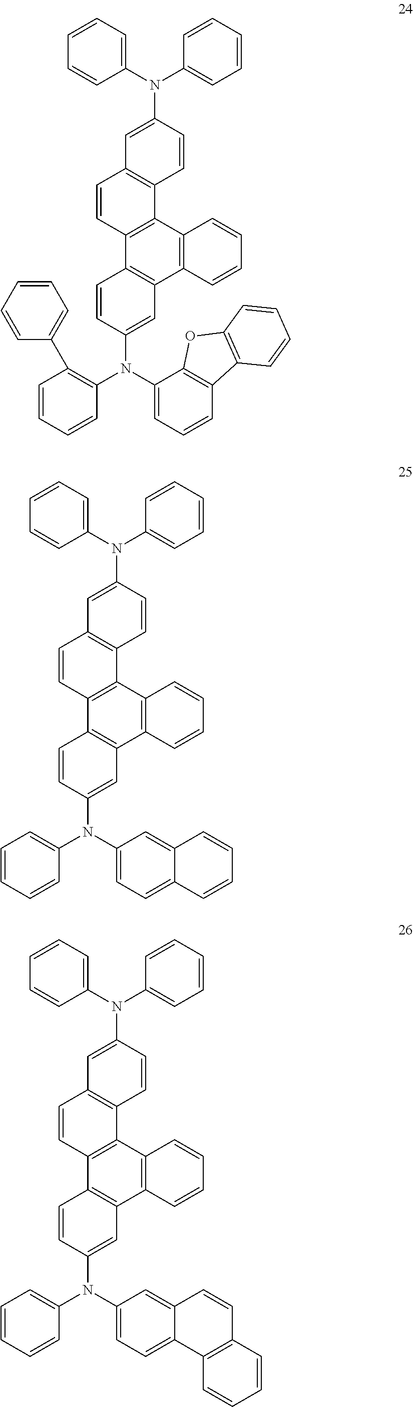 [SMILES: c1ccc(-c2ccccc2N(c2ccc3c(c2)c2ccccc2c2c4ccc(N(c5ccccc5)c5ccccc5)cc4ccc32)c2cccc3c2oc2ccccc23)cc1 ; c1ccc(N(c2ccccc2)c2ccc3c(ccc4c5ccc(N(c6ccccc6)c6ccc7ccccc7c6)cc5c5ccccc5c34)c2)cc1 ; c1ccc(N(c2ccccc2)c2ccc3c(ccc4c5ccc(N(c6ccccc6)c6ccc7c(ccc8ccccc87)c6)cc5c5ccccc5c34)c2)cc1]

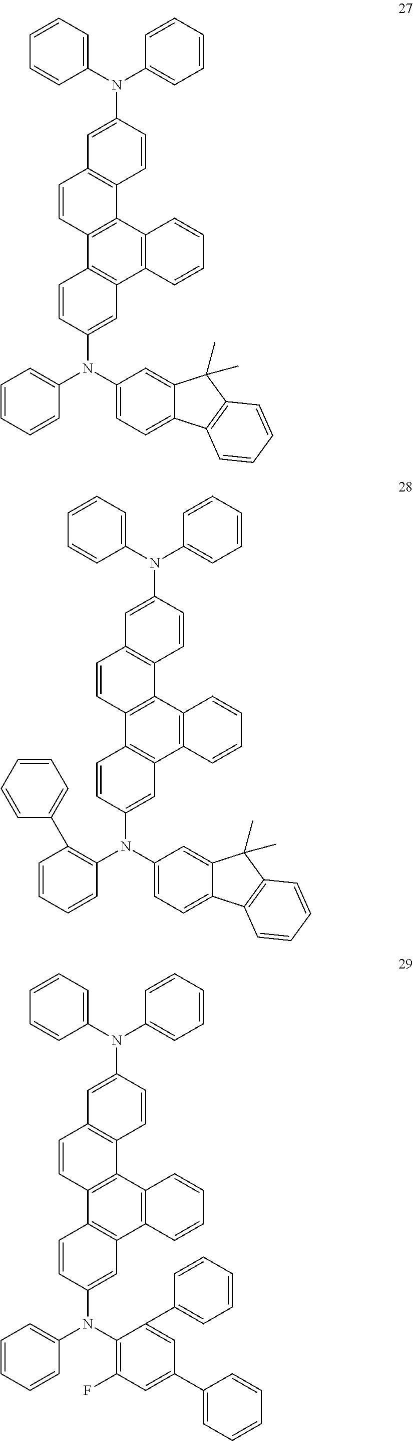

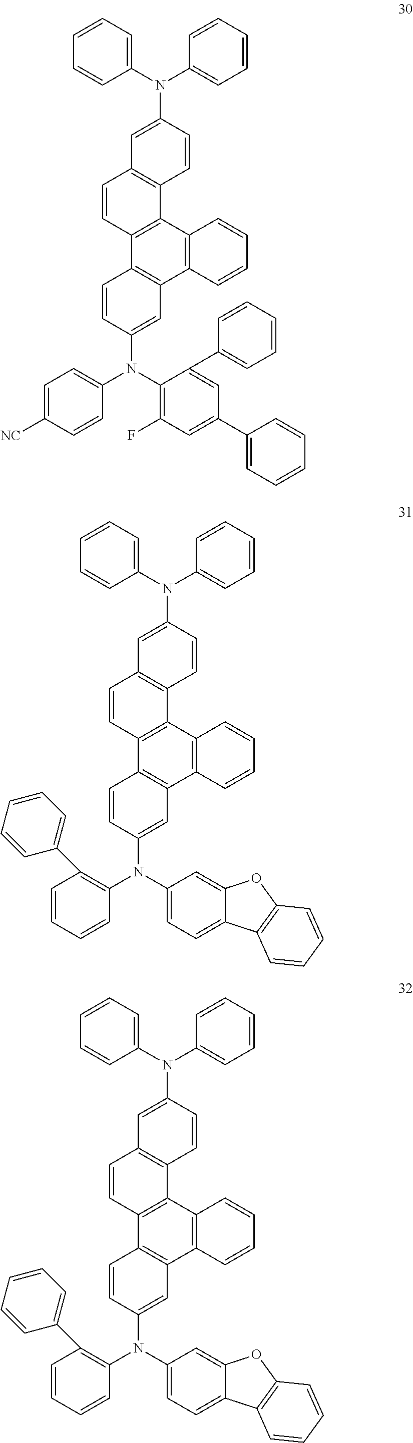 [SMILES: N#Cc1ccc(N(c2ccc3c(c2)c2ccccc2c2c4ccc(N(c5ccccc5)c5ccccc5)cc4ccc32)c2c(F)cc(-c3ccccc3)cc2-c2ccccc2)cc1 ; c1ccc(-c2ccccc2N(c2ccc3c(c2)oc2ccccc23)c2ccc3c(c2)c2ccccc2c2c4ccc(N(c5ccccc5)c5ccccc5)cc4ccc32)cc1 ; c1ccc(-c2ccccc2N(c2ccc3c(c2)oc2ccccc23)c2ccc3c(c2)c2ccccc2c2c4ccc(N(c5ccccc5)c5ccccc5)cc4ccc32)cc1]

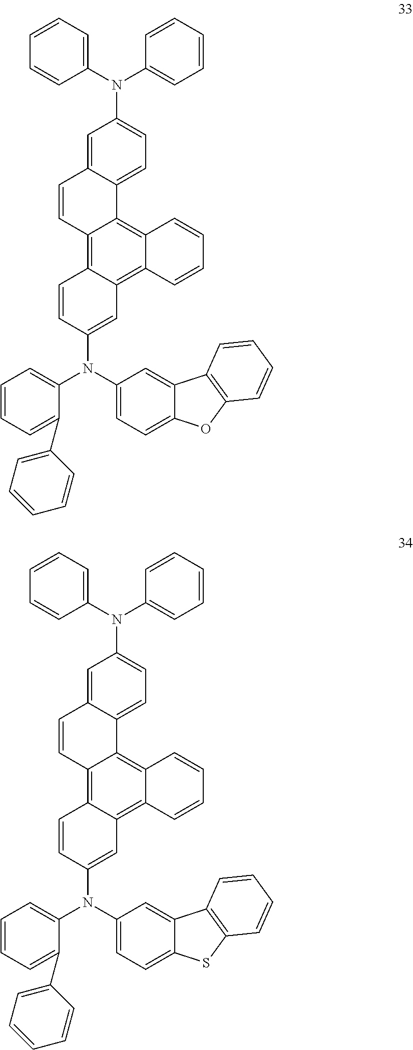

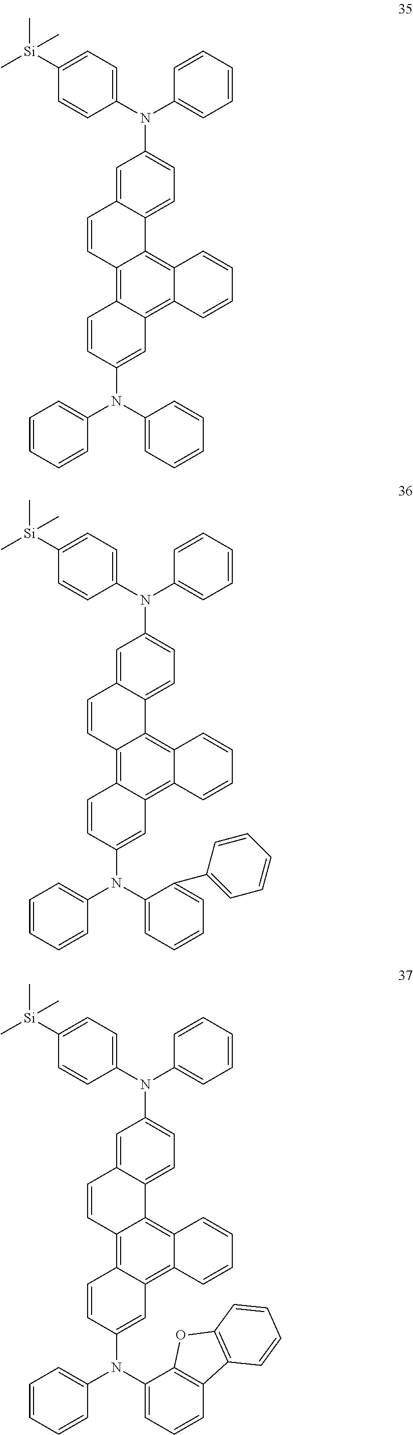

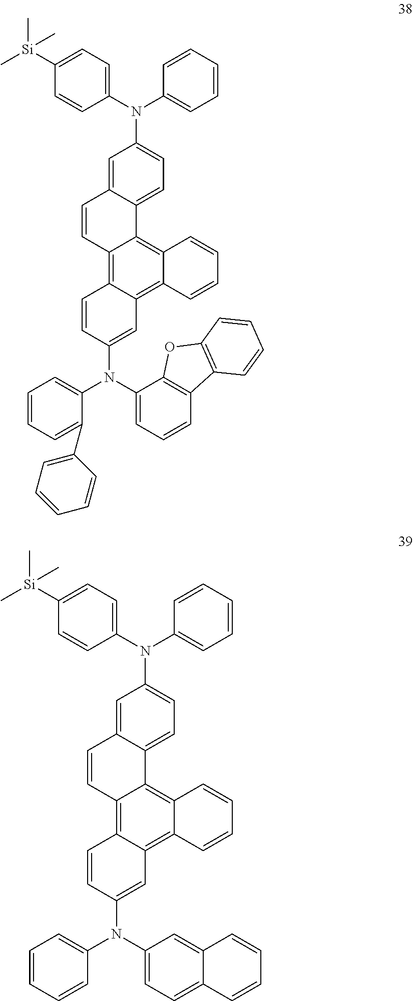 [SMILES: C[Si](C)(C)c1ccc(N(c2ccccc2)c2ccc3c(ccc4c5ccc(N(c6ccccc6-c6ccccc6)c6cccc7c6oc6ccccc67)cc5c5ccccc5c34)c2)cc1 ; C[Si](C)(C)c1ccc(N(c2ccccc2)c2ccc3c(ccc4c5ccc(N(c6ccccc6)c6ccc7ccccc7c6)cc5c5ccccc5c34)c2)cc1]

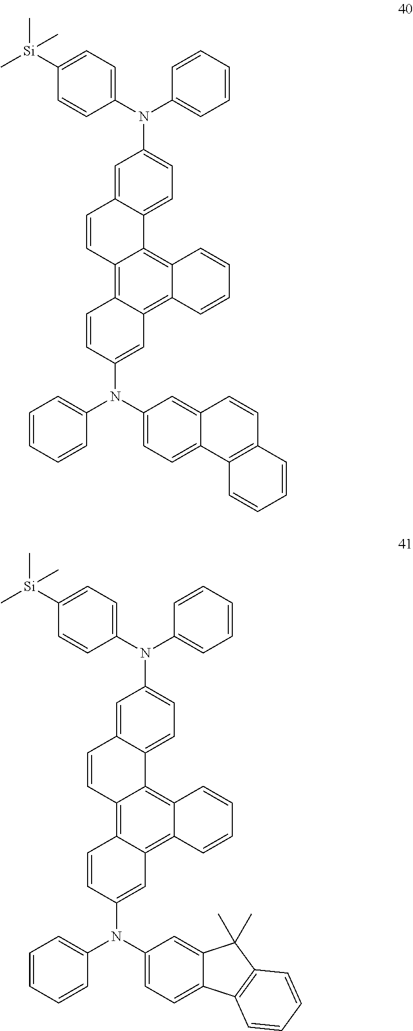 [SMILES: C[Si](C)(C)c1ccc(N(c2ccccc2)c2ccc3c(ccc4c5ccc(N(c6ccccc6)c6ccc7c(ccc8ccccc87)c6)cc5c5ccccc5c34)c2)cc1 ; CC1(C)c2ccccc2-c2ccc(N(c3ccccc3)c3ccc4c(c3)c3ccccc3c3c5ccc(N(c6ccccc6)c6ccc([Si](C)(C)C)cc6)cc5ccc43)cc21]

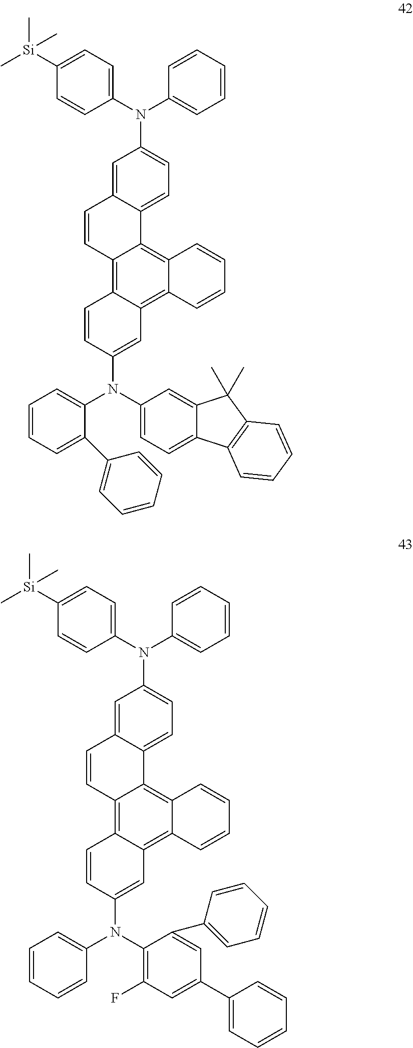 [SMILES: CC1(C)c2ccccc2-c2ccc(N(c3ccc4c(c3)c3ccccc3c3c5ccc(N(c6ccccc6)c6ccc([Si](C)(C)C)cc6)cc5ccc43)c3ccccc3-c3ccccc3)cc21 ; C[Si](C)(C)c1ccc(N(c2ccccc2)c2ccc3c(ccc4c5ccc(N(c6ccccc6)c6c(F)cc(-c7ccccc7)cc6-c6ccccc6)cc5c5ccccc5c34)c2)cc1]

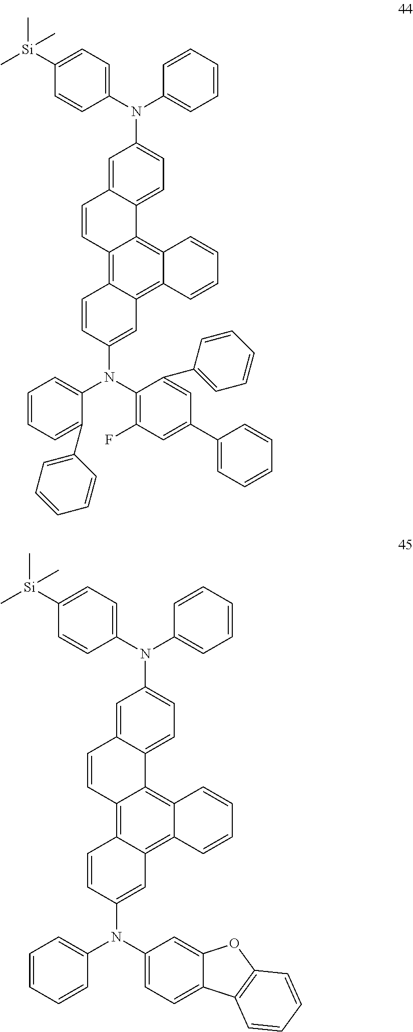 [SMILES: C[Si](C)(C)c1ccc(N(c2ccccc2)c2ccc3c(ccc4c5ccc(N(c6ccccc6-c6ccccc6)c6c(F)cc(-c7ccccc7)cc6-c6ccccc6)cc5c5ccccc5c34)c2)cc1 ; C[Si](C)(C)c1ccc(N(c2ccccc2)c2ccc3c(ccc4c5ccc(N(c6ccccc6)c6ccc7c(c6)oc6ccccc67)cc5c5ccccc5c34)c2)cc1]

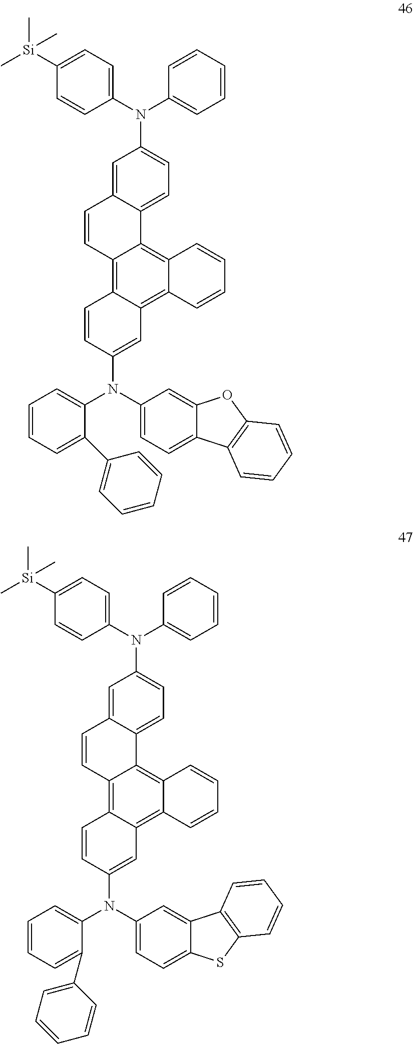 [SMILES: C[Si](C)(C)c1ccc(N(c2ccccc2)c2ccc3c(ccc4c5ccc(N(c6ccc7c(c6)oc6ccccc67)c6ccccc6-c6ccccc6)cc5c5ccccc5c34)c2)cc1 ; C[Si](C)(C)c1ccc(N(c2ccccc2)c2ccc3c(ccc4c5ccc(N(c6ccc7sc8ccccc8c7c6)c6ccccc6-c6ccccc6)cc5c5ccccc5c34)c2)cc1]

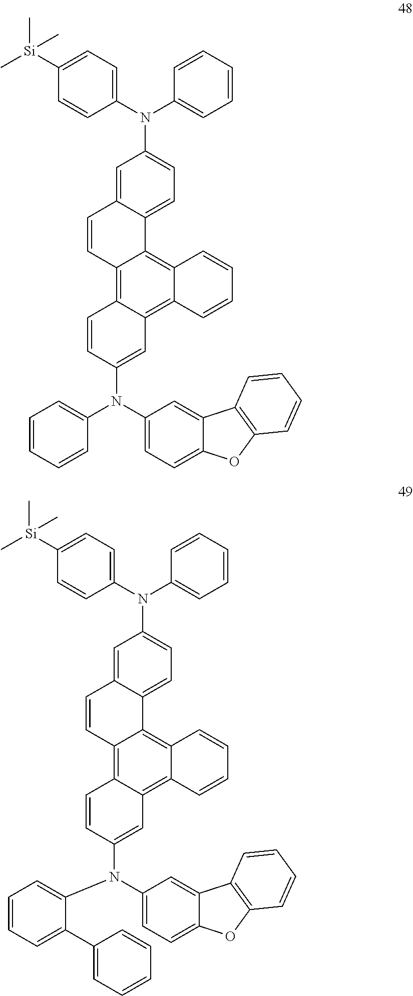

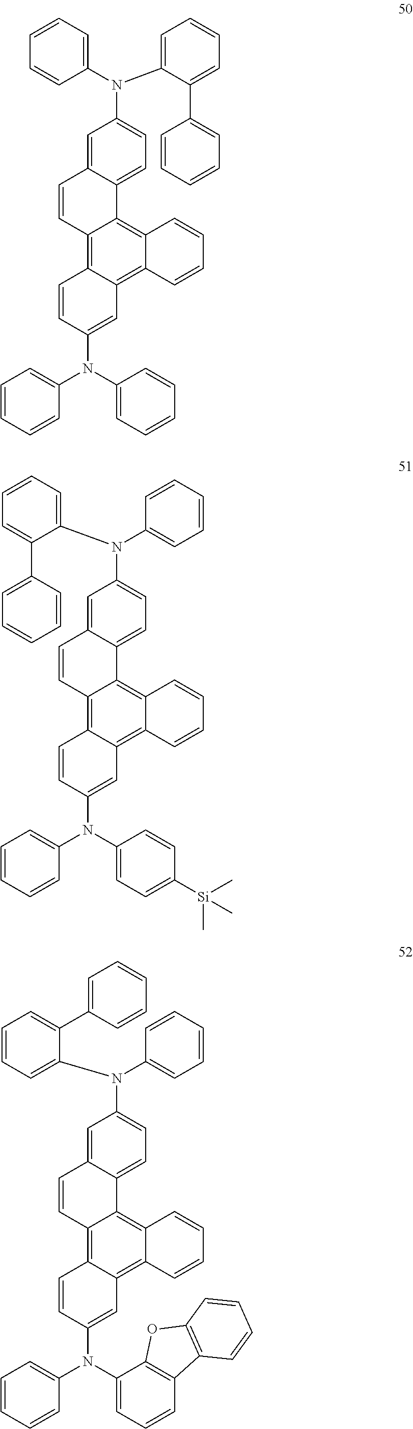

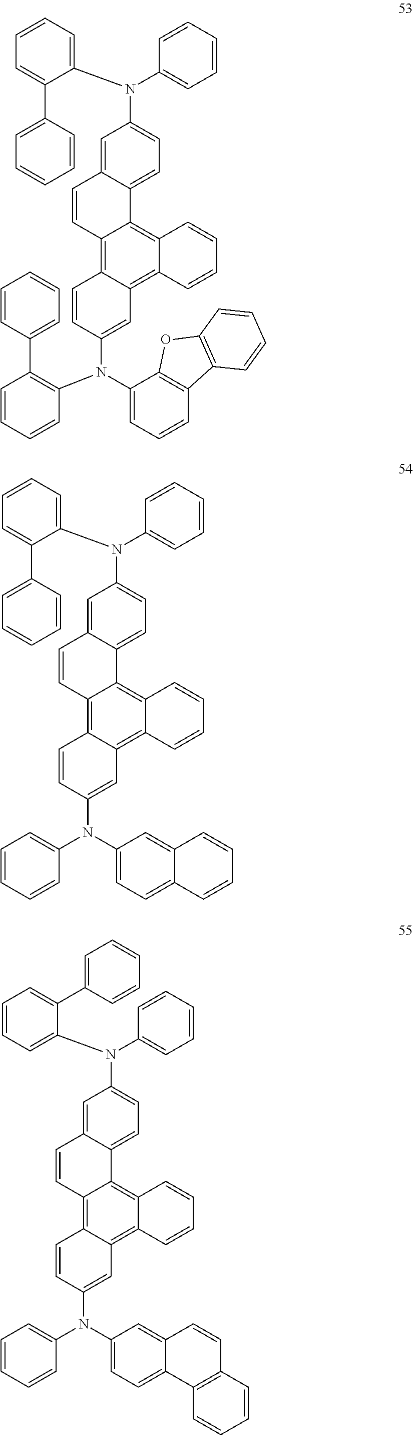

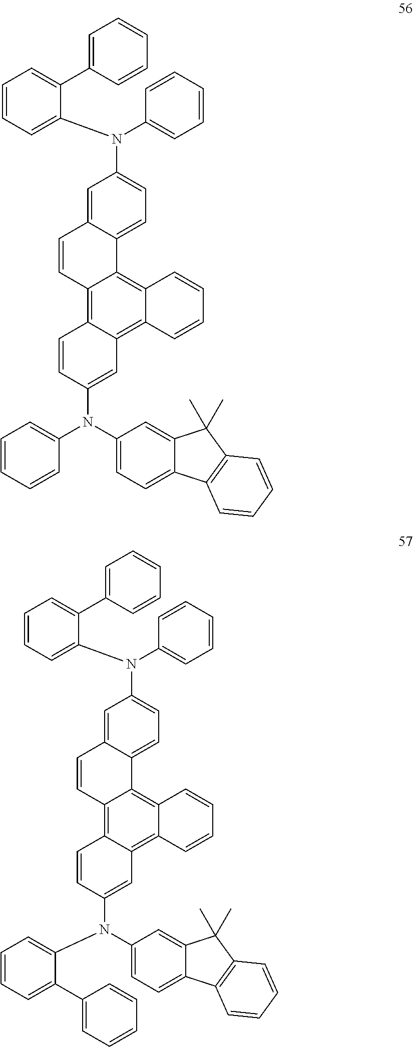 [SMILES: CC1(C)c2ccccc2-c2ccc(N(c3ccccc3)c3ccc4c(c3)c3ccccc3c3c5ccc(N(c6ccccc6)c6ccccc6-c6ccccc6)cc5ccc43)cc21 ; CC1(C)c2ccccc2-c2ccc(N(c3ccc4c(c3)c3ccccc3c3c5ccc(N(c6ccccc6)c6ccccc6-c6ccccc6)cc5ccc43)c3ccccc3-c3ccccc3)cc21]

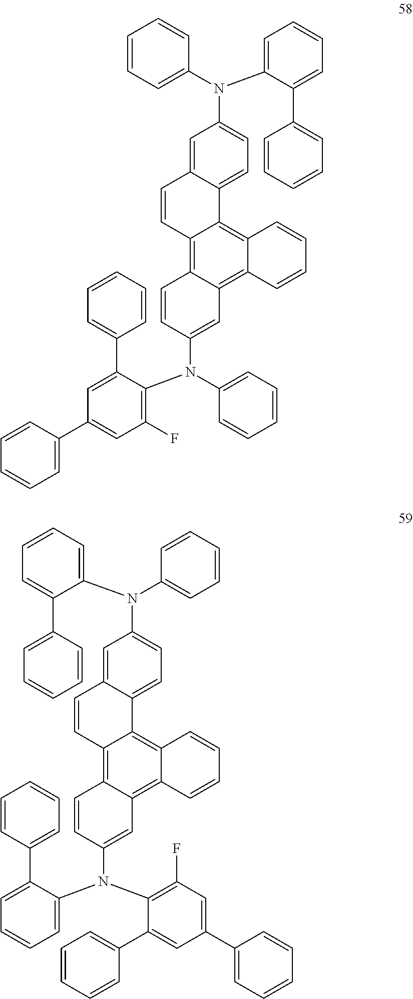

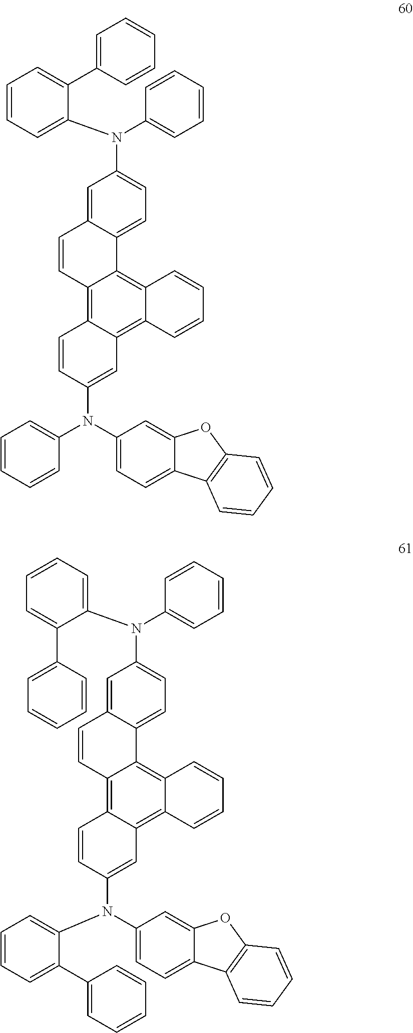 [SMILES: c1ccc(-c2ccccc2N(c2ccccc2)c2ccc3c(ccc4c5ccc(N(c6ccccc6)c6ccc7c(c6)oc6ccccc67)cc5c5ccccc5c34)c2)cc1 ; c1ccc(-c2ccccc2N(c2ccccc2)c2ccc3c(ccc4c5ccc(N(c6ccc7c(c6)oc6ccccc67)c6ccccc6-c6ccccc6)cc5c5ccccc5c34)c2)cc1]

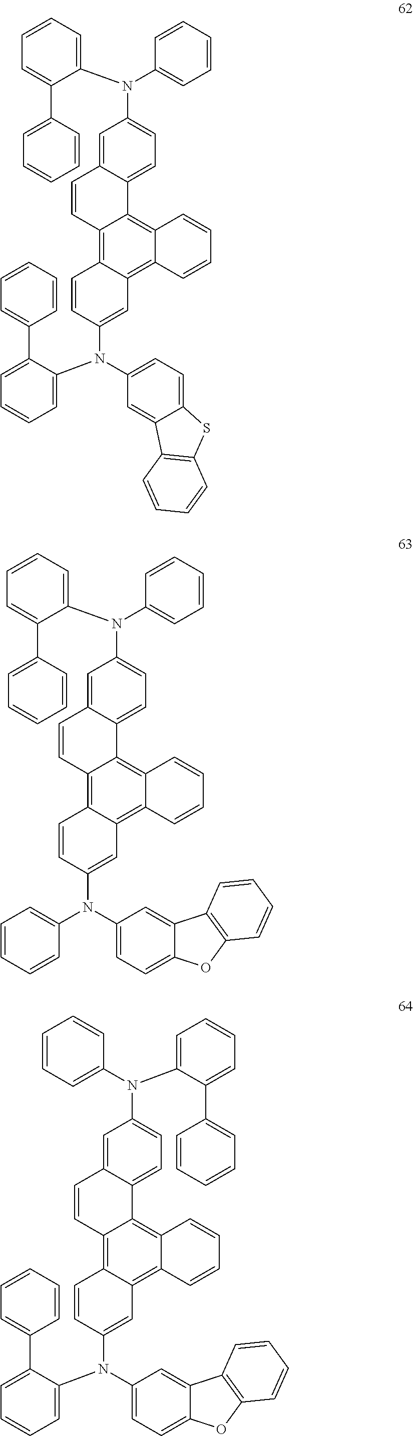

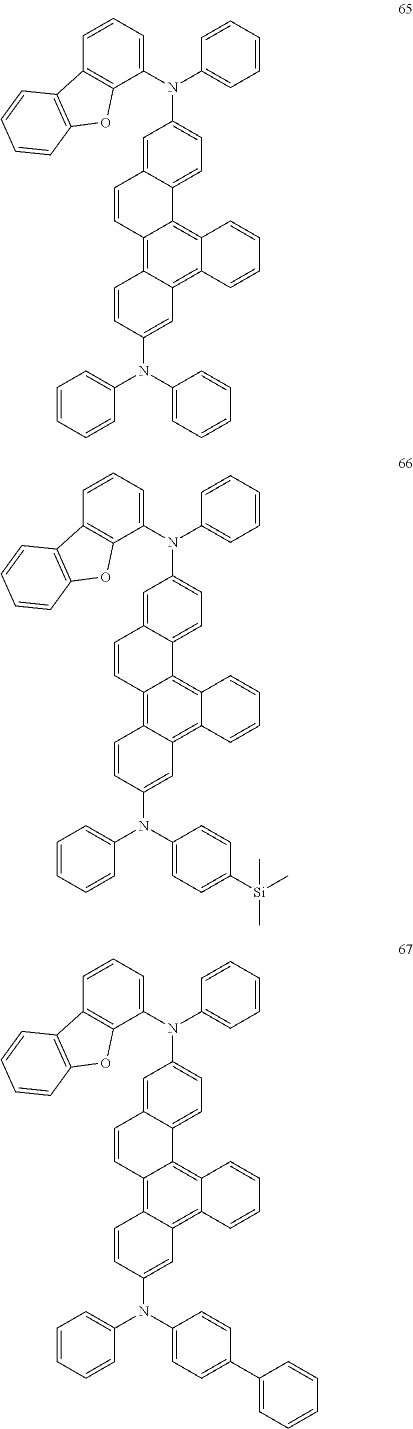

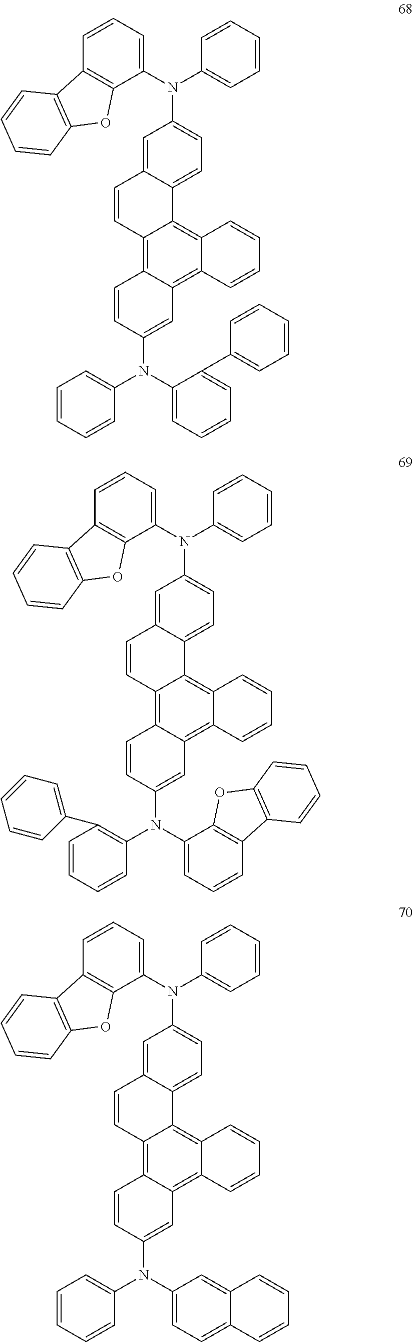

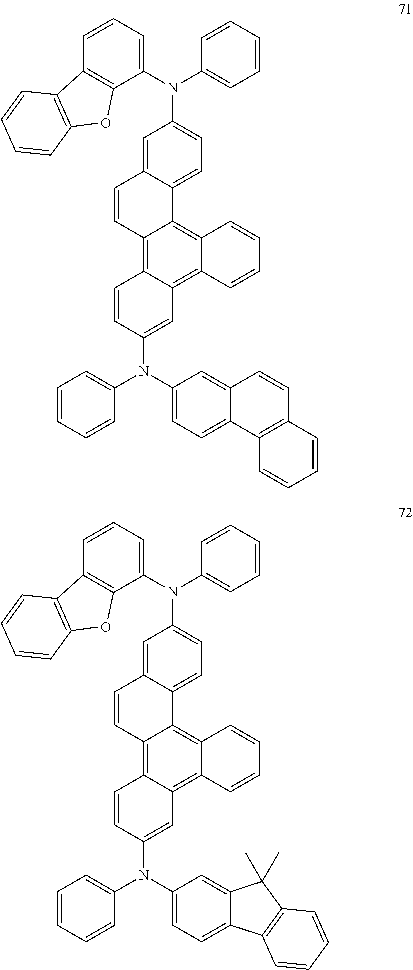 [SMILES: c1ccc(N(c2ccc3c(ccc4ccccc43)c2)c2ccc3c(c2)c2ccccc2c2c4ccc(N(c5ccccc5)c5cccc6c5oc5ccccc56)cc4ccc32)cc1 ; CC1(C)c2ccccc2-c2ccc(N(c3ccccc3)c3ccc4c(c3)c3ccccc3c3c5ccc(N(c6ccccc6)c6cccc7c6oc6ccccc67)cc5ccc43)cc21]

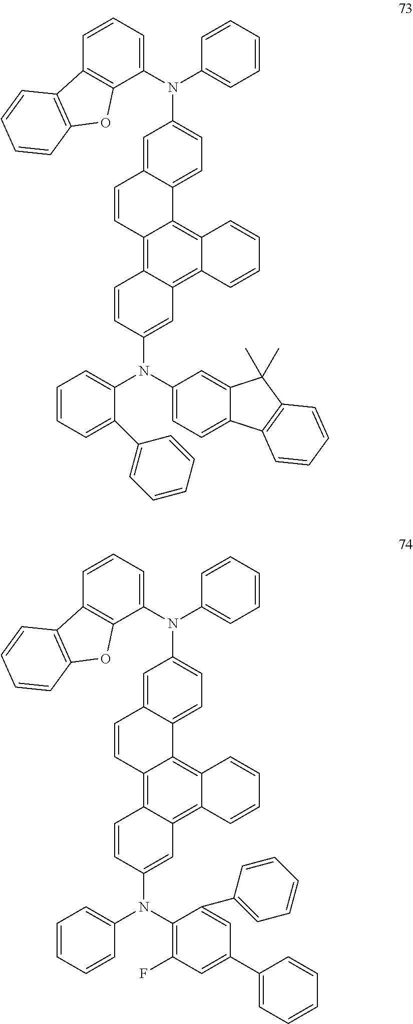 [SMILES: CC1(C)c2ccccc2-c2ccc(N(c3ccc4c(c3)c3ccccc3c3c5ccc(N(c6ccccc6)c6cccc7c6oc6ccccc67)cc5ccc43)c3ccccc3-c3ccccc3)cc21 ; Fc1cc(-c2ccccc2)cc(-c2ccccc2)c1N(c1ccccc1)c1ccc2c(c1)c1ccccc1c1c3ccc(N(c4ccccc4)c4cccc5c4oc4ccccc45)cc3ccc21]

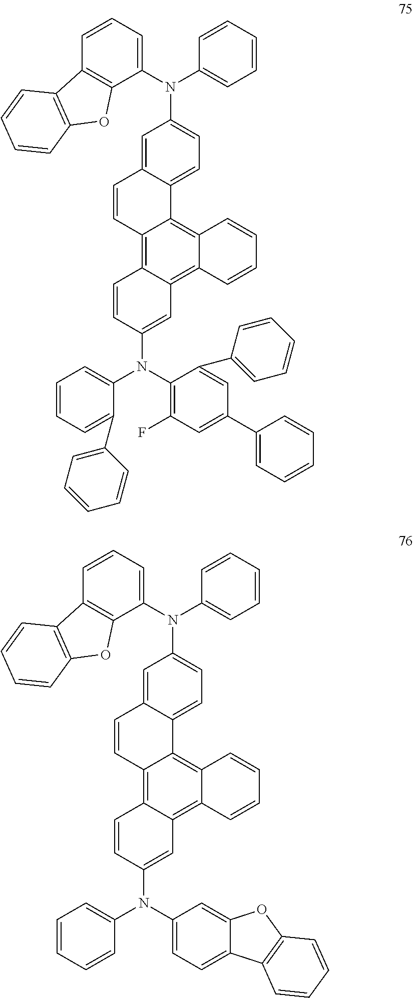

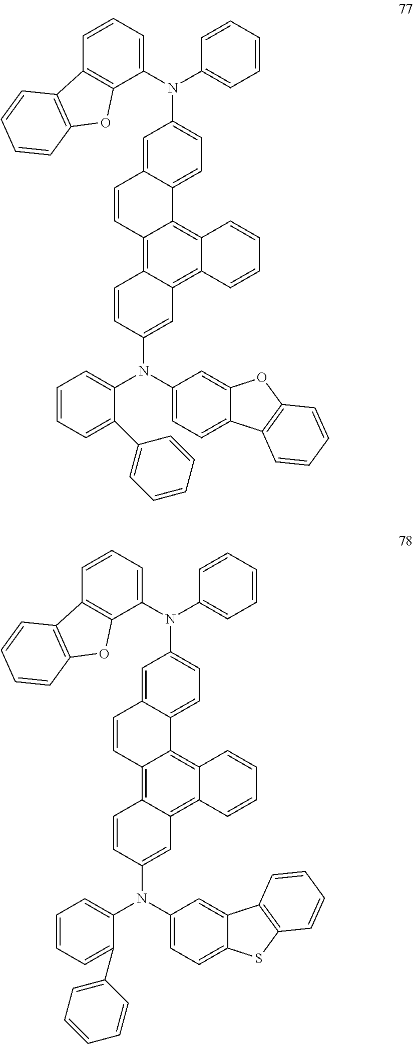 [SMILES: c1ccc(-c2ccccc2N(c2ccc3c(c2)oc2ccccc23)c2ccc3c(c2)c2ccccc2c2c4ccc(N(c5ccccc5)c5cccc6c5oc5ccccc56)cc4ccc32)cc1 ; c1ccc(-c2ccccc2N(c2ccc3sc4ccccc4c3c2)c2ccc3c(c2)c2ccccc2c2c4ccc(N(c5ccccc5)c5cccc6c5oc5ccccc56)cc4ccc32)cc1]

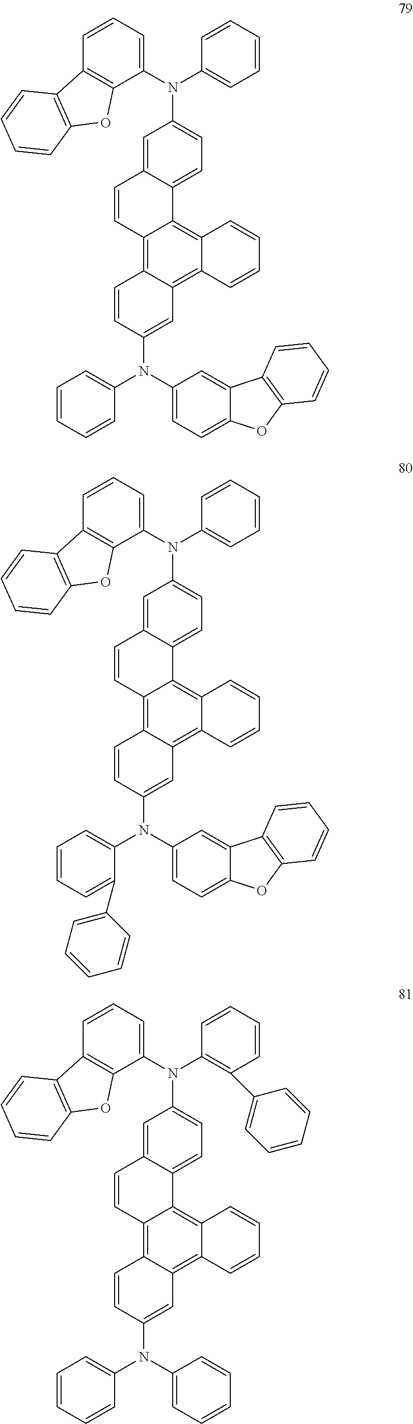

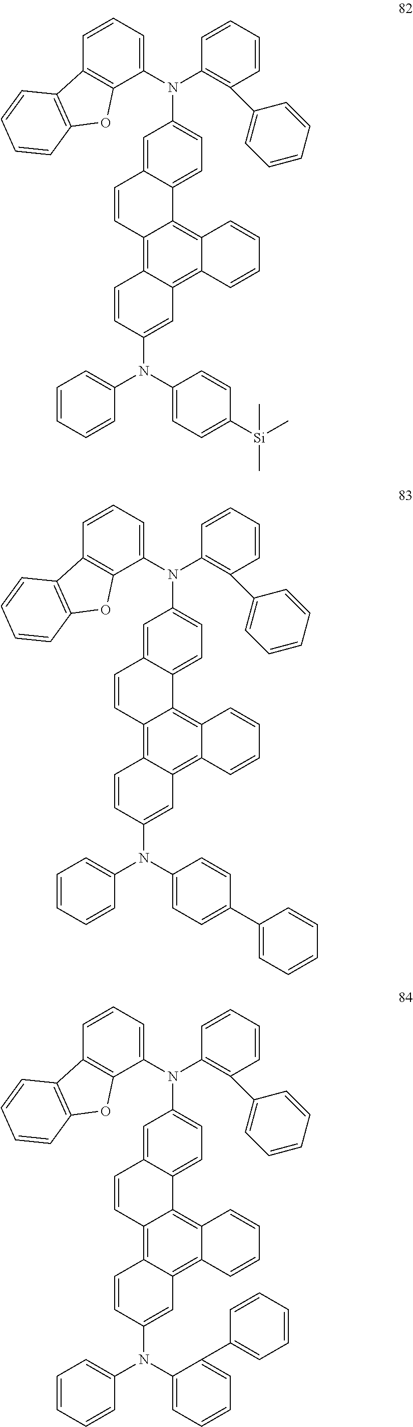 [SMILES: C[Si](C)(C)c1ccc(N(c2ccccc2)c2ccc3c(c2)c2ccccc2c2c4ccc(N(c5ccccc5-c5ccccc5)c5cccc6c5oc5ccccc56)cc4ccc32)cc1 ; c1ccc(-c2ccc(N(c3ccccc3)c3ccc4c(c3)c3ccccc3c3c5ccc(N(c6ccccc6-c6ccccc6)c6cccc7c6oc6ccccc67)cc5ccc43)cc2)cc1 ; c1ccc(-c2ccccc2N(c2ccccc2)c2ccc3c(c2)c2ccccc2c2c4ccc(N(c5ccccc5-c5ccccc5)c5cccc6c5oc5ccccc56)cc4ccc32)cc1]

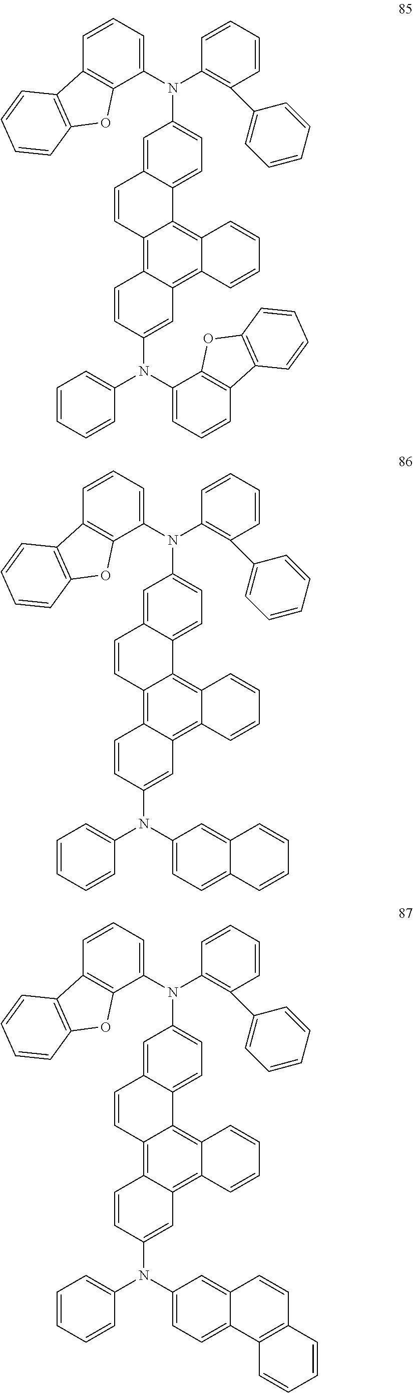

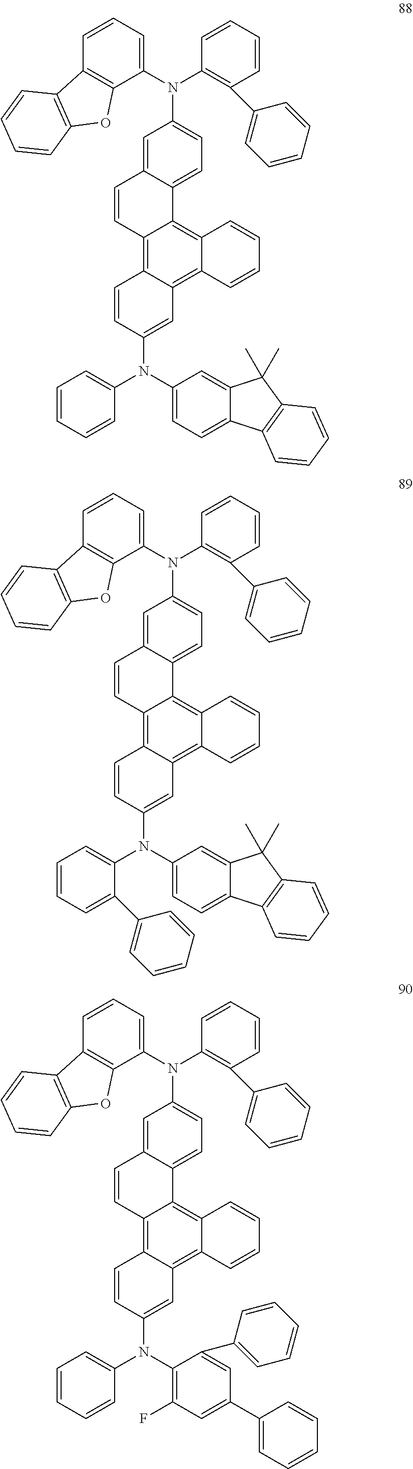

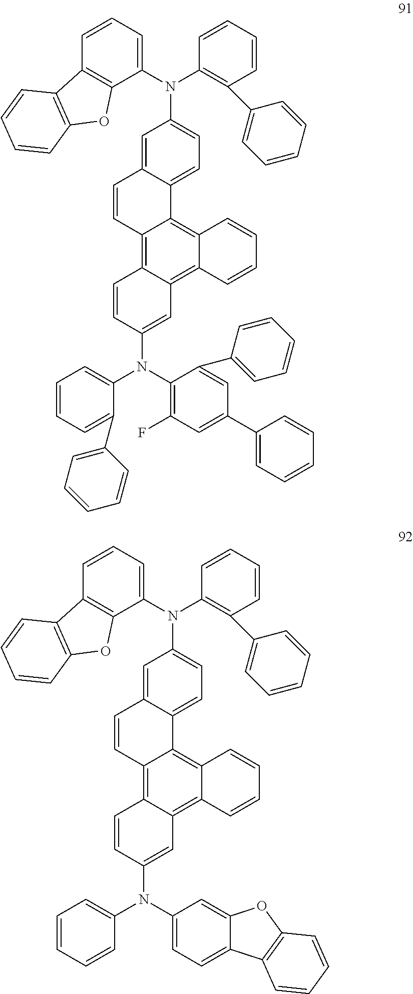

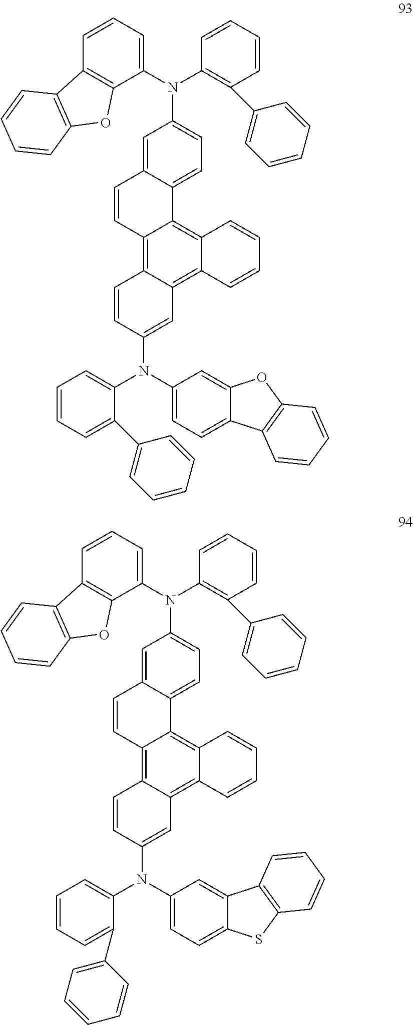 [SMILES: c1ccc(-c2ccccc2N(c2ccc3c(c2)oc2ccccc23)c2ccc3c(c2)c2ccccc2c2c4ccc(N(c5ccccc5-c5ccccc5)c5cccc6c5oc5ccccc56)cc4ccc32)cc1 ; c1ccc(-c2ccccc2N(c2ccc3sc4ccccc4c3c2)c2ccc3c(c2)c2ccccc2c2c4ccc(N(c5ccccc5-c5ccccc5)c5cccc6c5oc5ccccc56)cc4ccc32)cc1]

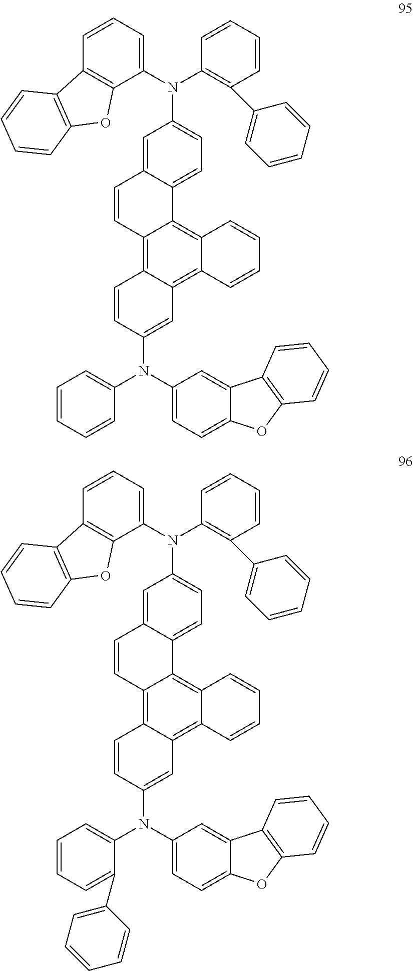

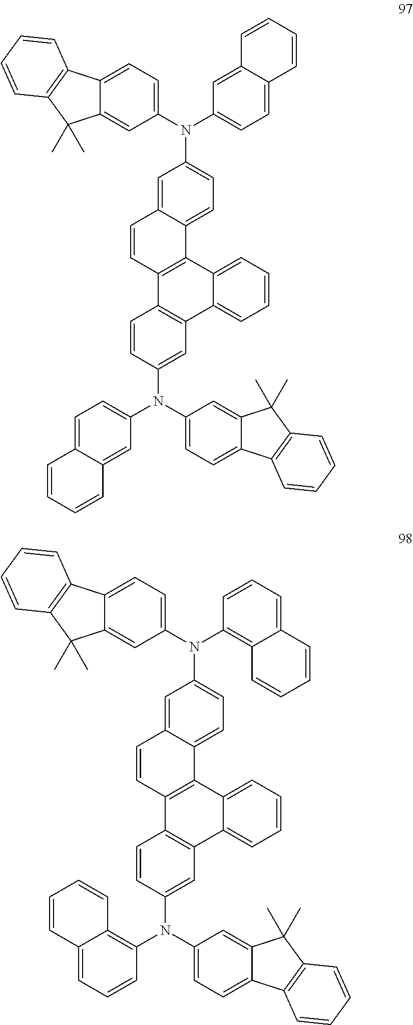

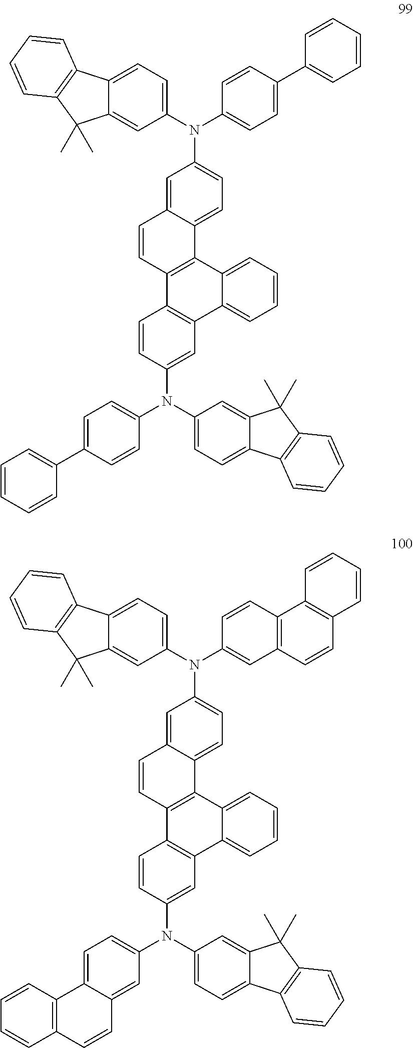 [SMILES: CC1(C)c2ccccc2-c2ccc(N(c3ccc(-c4ccccc4)cc3)c3ccc4c(ccc5c6ccc(N(c7ccc(-c8ccccc8)cc7)c7ccc8c(c7)C(C)(C)c7ccccc7-8)cc6c6ccccc6c45)c3)cc21 ; CC1(C)c2ccccc2-c2ccc(N(c3ccc4c(ccc5ccccc54)c3)c3ccc4c(ccc5c6ccc(N(c7ccc8c(c7)C(C)(C)c7ccccc7-8)c7ccc8c(ccc9ccccc98)c7)cc6c6ccccc6c45)c3)cc21]

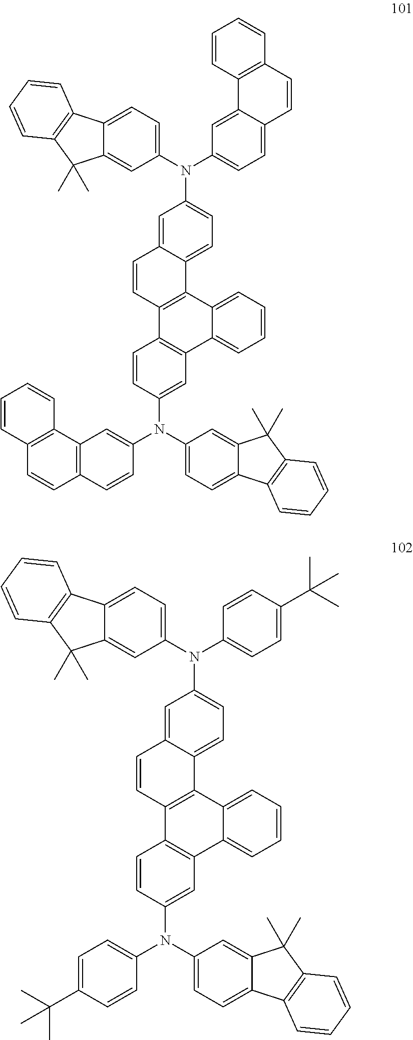

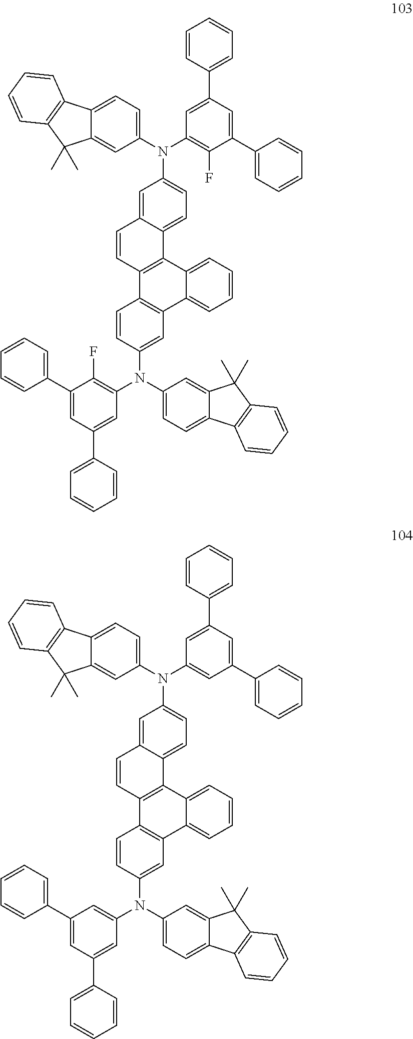 [SMILES: CC1(C)c2ccccc2-c2ccc(N(c3ccc4c(ccc5c6ccc(N(c7ccc8c(c7)C(C)(C)c7ccccc7-8)c7cc(-c8ccccc8)cc(-c8ccccc8)c7F)cc6c6ccccc6c45)c3)c3cc(-c4ccccc4)cc(-c4ccccc4)c3F)cc21 ; CC1(C)c2ccccc2-c2ccc(N(c3cc(-c4ccccc4)cc(-c4ccccc4)c3)c3ccc4c(ccc5c6ccc(N(c7cc(-c8ccccc8)cc(-c8ccccc8)c7)c7ccc8c(c7)C(C)(C)c7ccccc7-8)cc6c6ccccc6c45)c3)cc21]

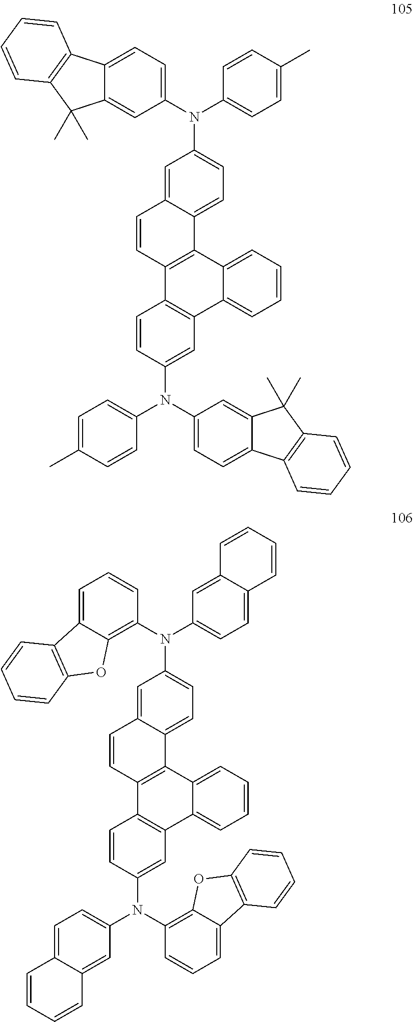 [SMILES: Cc1ccc(N(c2ccc3c(c2)C(C)(C)c2ccccc2-3)c2ccc3c(ccc4c5ccc(N(c6ccc(C)cc6)c6ccc7c(c6)C(C)(C)c6ccccc6-7)cc5c5ccccc5c34)c2)cc1 ; c1ccc2cc(N(c3ccc4c(ccc5c6ccc(N(c7ccc8ccccc8c7)c7cccc8c7oc7ccccc78)cc6c6ccccc6c45)c3)c3cccc4c3oc3ccccc34)ccc2c1]

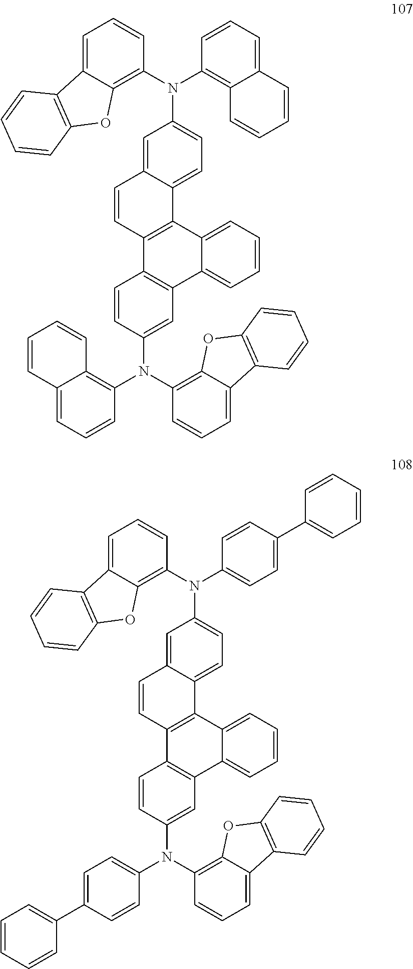 [SMILES: c1ccc2c(N(c3ccc4c(ccc5c6ccc(N(c7cccc8ccccc78)c7cccc8c7oc7ccccc78)cc6c6ccccc6c45)c3)c3cccc4c3oc3ccccc34)cccc2c1 ; c1ccc(-c2ccc(N(c3ccc4c(ccc5c6ccc(N(c7ccc(-c8ccccc8)cc7)c7cccc8c7oc7ccccc78)cc6c6ccccc6c45)c3)c3cccc4c3oc3ccccc34)cc2)cc1]

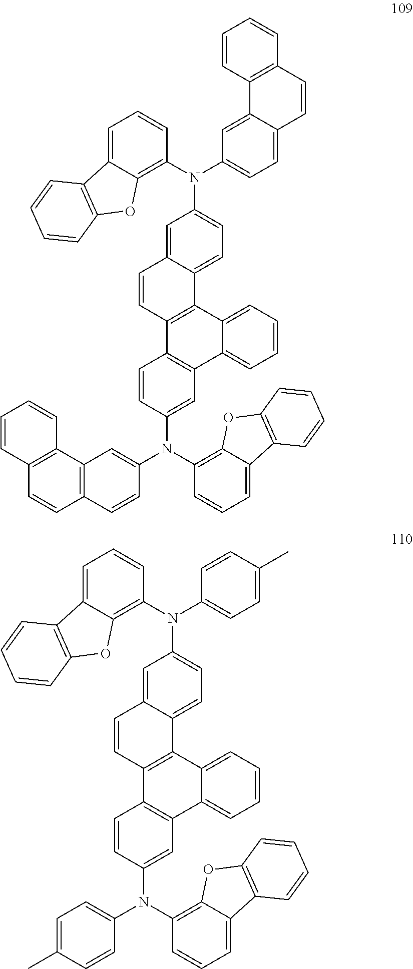

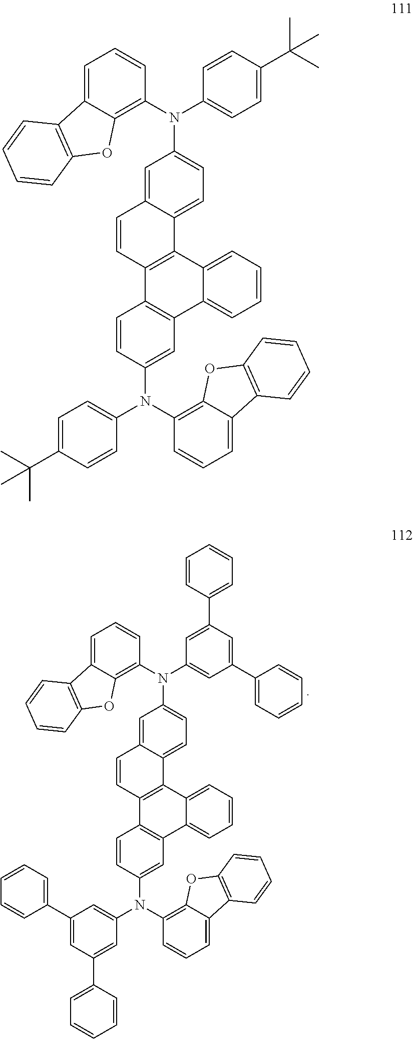 [SMILES: CC(C)(C)c1ccc(N(c2ccc3c(ccc4c5ccc(N(c6ccc(C(C)(C)C)cc6)c6cccc7c6oc6ccccc67)cc5c5ccccc5c34)c2)c2cccc3c2oc2ccccc23)cc1 ; c1ccc(-c2cc(-c3ccccc3)cc(N(c3ccc4c(ccc5c6ccc(N(c7cc(-c8ccccc8)cc(-c8ccccc8)c7)c7cccc8c7oc7ccccc78)cc6c6ccccc6c45)c3)c3cccc4c3oc3ccccc34)c2)cc1]

10. An organic light-emitting device comprising:
a first electrode;
a second electrode facing the first electrode; and
an organic layer between the first electrode and the second electrode, the organic layer comprising an emission layer,
wherein the organic layer comprises the compound of claim 1.

11. The organic light-emitting device of claim 10, wherein the first electrode is an anode, the second electrode is a cathode, and the organic layer comprises:
a hole transport region between the first electrode and the emission layer, the hole transport region comprising at least one selected from a hole injection layer, a hole transport layer, and an electron blocking layer; and
an electron transport region between the emission layer and the second electrode, the electron transport region comprising at least one selected from a hole blocking layer, an electron transport layer, and an electron injection layer.

12. The organic light-emitting device of claim 11, wherein the emission layer comprises the compound of Formula 1.

13. The organic light-emitting device of claim 11, wherein the emission layer comprises the compound of Formula 1 as a dopant.

14. The organic light-emitting device of claim 11, wherein the hole transport region comprises a charge-generation material.

15. The organic light-emitting device of claim 11, wherein the hole transport region comprises a p-dopant.

16. The organic light-emitting device of claim 11, wherein the hole transport region comprises at least one selected from a quinone derivative, a metal oxide, and a cyano group-containing compound.

17. The organic light-emitting device of claim 11, wherein the electron transport region comprises a metal complex.

18. The organic light-emitting device of claim 17, wherein the metal complex is a lithium (Li) complex.

19. The organic light-emitting device of claim 17, wherein the metal complex is a lithium quinolate (LiQ).

20. The organic light-emitting device of claim 17, wherein the metal complex is Compound ET-D2 below:

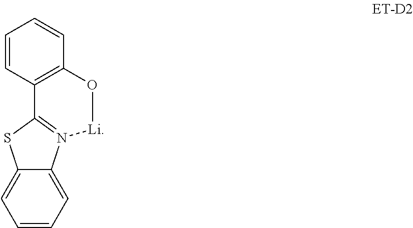

21. A flat display apparatus comprising the organic light-emitting device of claim 10, wherein the first electrode of the organic light-emitting device is electrically connected to a source electrode or a drain electrode of a thin film transistor.
